(19) United States
(12) Patent Application Publication
(10) Pub. No.: US 2024/0166720 A1
(43) Pub. Date: May 23, 2024

(54) BIOENGINEERED IMMUNOMODULATORY FUSION PROTEIN COMPOSITIONS

(71) Applicant: Janssen Biotech, Inc., Horsham, PA (US)

(72) Inventors: Ninkka TAMOT, Colmar, PA (US); Paul B. HARVILLA, Nazareth, PA (US); Douglas YAMADA, Ardmore, PA (US); Manuel Alejandro SEPULVEDA, West Windsor, NJ (US); Rajkumar GANESAN, Thousand Oaks, CA (US); Sanjaya SINGH, Blue Bell, PA (US)

(21) Appl. No.: 18/549,795

(22) PCT Filed: Mar. 11, 2022
(86) PCT No.: PCT/US2022/019926

§ 371 Date: Sep. 8, 2023

Related U.S. Application Data
(60) Provisional application No. 63/160,691, filed on Mar. 12, 2021.
(60) Provisional application No. 63/160,694, filed on Mar. 12, 2021.
(60) Provisional application No. 63/160,686, filed on Mar. 12, 2021.
(60) Provisional application No. 63/160,693, filed on Mar. 12, 2021.
(60) Provisional application No. 63/160,688, filed on Mar. 12, 2021.

(51) Int. Cl. C07K 14/705; A61K 39/00; A61K 39/39; A61P 37/04

(52) U.S. Cl. CPC C07K 14/70575; A61K 39/39; A61P 37/04; A61K 2039/55516; C07K 2319/00; C07K 2319/30

(57) ABSTRACT

Provided herein are bioengineered immunomodulatory fusion proteins and uses thereof for modulating immune responses, as well as uses for improving a response of a subject to a vaccine, or uses for treating a disease or disorder, such as cancer or a pathogen infection. Provided herein is a single chain trimeric CD40L Fc fusion protein comprising (a) three CD40 ligand CD40L subunits covalently linked to one another by peptide linkers (CD40L trimer); and (b) an Fc monomer peptide.

### CROSS-REFERENCE TO RELATED APPLICATIONS

This application claims the benefit of U.S. Ser. No. 63/160,686, filed Mar. 12, 2021; U.S. Ser. No. 63/160,688, filed Mar. 12, 2021; U.S. Ser. No. 63/160,691, filed Mar. 12, 2021; U.S. Ser. No. 63/160,693, filed Mar. 12, 2021; U.S. Ser. No. 63/160,694, filed Mar. 12, 2021, each of which is herein incorporated by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file “14620-628-228_SEQ_LISTING” and a creation date of Mar. 6, 2022 and having a size of 150,953 bytes. The sequence listing submitted via EFS-Web is part of the specification and is herein incorporated by reference in its entirety.

### 1. FIELD

Provided herein, in some embodiments, are materials and methods for bioengineered immunomodulatory fusion proteins and uses thereof for detecting, modulating immune responses and/or immune related conditions, as well as materials and methods for screening, diagnosing, influencing, improving a response of a subject to a pathogen or vaccine, or treating a disease or disorder, such as neoplasia, hyperplasia, cancers or a pathogen infection(s).

### 2. BACKGROUND

Immunity is a multifaceted host response involving various molecules. Manzoor Ahmad Mir, Chapter 1—Introduction to Costimulation and Costimulatory Molecules, Editor(s): Manzoor Ahmad Mir, Developing Costimulatory Molecules for Immunotherapy of Diseases, Academic Press, 2015, Pages 1-43, ISBN 9780128025857. One such molecule involved in an immune response is CD40 ligand (CD40L) (CD154), a Tumor Necrosis Factor (TNF) super family member that is present in various cell and tissue types, and is highly expressed on CD4+ T cells and interacts with the CD40 receptor polypeptide to enhance costimulatory signaling. The CD40 receptor polypeptide is expressed on a variety of innate and adaptive cells including dendritic and B cells. Adv Drug Deliv Rev. 2019, 15; 141:92-103; Immunol Rev. 2009 May; 229(1). CD40 agonism may enhance antigen presentation and is a suitable target for monoclonal antibody applications, such as diagnostic, prognostic and therapy. Cancers (Basel). 2021 Mar. 15; 13(6):1302.

### 3. SUMMARY

The inventors of the present invention, with the understanding that inter alia CD40 agonist monoclonal antibodies perform poorly in the absence of Fc crosslinking and have shown little clinical benefit, and furthermore, systemic administration of CD40 agonist antibodies has been linked to adverse events and to hepatic toxicity, identified and addressed unmet needs in the art for improved constructs of engineered CD40 agonists for modulating immune responses, as well as improving a response of a subject to a vaccine, or treating a disease or disorder, such as cancer or a pathogen infection (e.g., a viral infection), for example. Accordingly, in one aspect, provided herein is a single chain trimeric CD40L Fc fusion protein comprising (a) three CD40 ligand CD40L subunits covalently linked to one another by peptide linkers (CD40L trimer); and (b) an Fc monomer peptide.

In some embodiments, the Fc monomer peptide is covalently linked to the CD40L trimer by a peptide tether. In some embodiments, the peptide tether comprises between 0 and 20 amino acids.

In some embodiments, the CD40 ligand subunits comprise a portion of the CD40L extracellular domain.

In some embodiments, the CD40L trimer is connected to the N-terminus of the Fc monomer peptide. In some embodiments, the single chain trimeric CD40L Fc fusion protein comprises any one sequence selected from SEQ ID NOS:1-12, or a fragment thereof.

In some embodiments, the CD40L trimer is connected to the C-terminus of the Fc monomer peptide. In some embodiments, the single chain trimeric CD40L Fc fusion protein comprises any one sequence selected from SEQ ID NOS:13-19, or a fragment thereof. In some embodiments, the single chain trimeric CD40L Fc fusion protein comprises SEQ ID NO:16 or a fragment thereof.

In some embodiments, the CD40 ligand subunits comprise any one of the sequences selected from SEQ ID NOS:20-22, or a fragment thereof.

In some embodiments, the Fc monomer peptide comprises a human Fc sequence. In some embodiments, the human Fc sequence comprises a sequence selected from immunoglobulins IgG, IgA, IgM, IgD and IgE. In some embodiments, the human Fc sequence comprises an IgG sequence. In some embodiments, the IgG sequence is selected from IgG1, IgG2, IgG3 and IgG4. In some embodiments, the IgG sequence comprises an IgG1 sequence. In some embodiments, the IgG1 sequence comprises SEQ ID NOS:30 or 31, or a fragment thereof. In some embodiments, the IgG sequence comprises an IgG2 sequence. In some embodiments, the IgG2 sequence comprises SEQ ID NO:29 or a fragment thereof.

In some embodiments, the peptide linker is selected from the group comprising of EGKSSGSGS (SEQ ID NO:23) and (G₃S)₃ (SEQ ID NO:25).

In some embodiments, the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).

In some embodiments, the single chain trimeric CD40L Fc fusion protein enhances activation of a CD40 polypeptide compared to wild-type CD40L. In some embodiments, the activation of the CD40 polypeptide enhances the immune-stimulatory functions of dendritic cells, B cells, monocytes and macrophages. In some embodiments, the activation of the CD40 polypeptide comprises enhanced T cell activation compared to wild-type CD40L. In some embodiments, the activation of the CD40 polypeptide comprises enhanced dendritic cell activation compared to wild-type CD40L. In some embodiments, the single chain trimeric CD40L Fc fusion protein enhances anti-tumor activity compared to wild-type CD40L.

In another aspect, provided herein is a dimer comprising two single chain trimeric CD40L Fc fusion proteins disclosed herein. In some embodiments, the dimer is a homodimer. In some embodiments, the dimer is formed by association of the Fc monomer peptides.

In yet another aspect, provided herein is a polynucleotide encoding a single chain trimeric CD40L Fc fusion protein comprising (a) three CD40L subunits covalently linked to one another by peptide linkers; and (b) an Fc monomer peptide.

In yet another aspect, provided herein is a vector comprising a polynucleotide encoding a single chain trimeric CD40L fusion protein comprising (a) three CD40L subunits covalently linked to one another by peptide linkers; and (b) an Fc monomer peptide.

In yet another aspect, provided herein is a host cell comprising a vector comprising a polynucleotide encoding a single chain trimeric CD40L fusion protein comprising (a) three CD40L subunits covalently linked to one another by peptide linkers; and (b) an Fc monomer peptide.

In yet another aspect, provided herein is a host cell comprising a polynucleotide encoding a single chain trimeric CD40L fusion protein comprising (a) three CD40L subunits covalently linked to one another by peptide linkers; and (b) an Fc monomer peptide.

In yet another aspect, provided herein is a pharmaceutical composition comprising a pharmaceutically acceptable carrier and a single chain trimeric CD40L fusion protein comprising (a) three CD40L subunits covalently linked to one another by peptide linkers; and (b) an Fc monomer peptide.

In yet another aspect, provided herein is a kit comprising a single chain trimeric CD40L fusion protein comprising (a) three CD40L subunits covalently linked to one another by peptide linkers; and (b) an Fc monomer peptide.

In one aspect, provided herein is a system comprising a means for providing a single chain trimeric CD40L fusion protein comprising (a) three CD40L subunits covalently linked to one another by peptide linkers; and (b) an Fc monomer peptide. In another aspect, provided herein is a system comprising a means for providing a dimer comprising two single chain trimeric CD40L Fc fusion proteins, each comprising (a) three CD40L subunits covalently linked to one another by peptide linkers; and (b) an Fc monomer peptide.

In one aspect, provided herein is a method of activating a CD40 polypeptide comprising contacting the CD40 polypeptide with the single chain trimeric CD40L Fc fusion protein disclosed herein, wherein said single chain trimeric CD40L Fc fusion protein activates the CD40 polypeptide upon binding. In another aspect, provided herein is a method of activating a CD40 polypeptide comprising contacting the CD40 polypeptide with a dimer comprising two single chain trimeric CD40L Fc fusion proteins disclosed herein, wherein said single chain trimeric CD40L Fc fusion protein dimer activates the CD40 polypeptide upon binding. In yet another aspect, provided herein is a method of activating a T-cell comprising contacting the T-cell with an antigen presenting cell in the presence of the single chain trimeric CD40L Fc fusion protein disclosed herein, wherein the antigen presenting cell expresses a CD40 polypeptide, and wherein said single chain trimeric CD40L Fc fusion protein activates the T-cell upon binding the CD40 polypeptide. In yet another aspect, provided herein is a method of activating a T cell comprising contacting the T-cell with an antigen presenting cell in the presence of a dimer comprising two single chain trimeric CD40L Fc fusion proteins disclosed herein, wherein the antigen presenting cell expresses a CD40 polypeptide, and wherein said single chain trimeric CD40L Fc fusion protein dimer activates the T-cell upon binding the CD40 polypeptide. In some embodiments, said antigen presenting cell presents an antigen to the T cell. In yet another aspect, provided herein is a method of activating a dendritic cell comprising contacting the CD40 polypeptide with the single chain trimeric CD40L Fc fusion protein disclosed herein, wherein said single chain trimeric CD40L Fc fusion protein activates the dendritic cell upon binding the CD40 polypeptide. In yet another aspect, provided herein is a method of activating a dendritic cell comprising contacting the CD40 polypeptide with a dimer comprising two single chain trimeric CD40L Fc fusion proteins disclosed herein, wherein said single chain trimeric CD40L Fc fusion protein dimer activates the dendritic cell upon binding the CD40 polypeptide.

In some embodiments, the method is performed in vitro. In some embodiments, the method is performed in vivo. In some embodiments, the contacting further comprises administering a pharmaceutical composition comprising a pharmaceutically acceptable carrier and the single chain trimeric CD40L Fc fusion proteins. In some embodiments, the contacting enhances an innate anti-neoplastic immune response.

In one aspect, provided herein is a method of treating cancer in a subject comprising administering to the subject a therapeutically effective amount of the single chain trimeric CD40L Fc fusion protein disclosed herein. In another aspect, provided herein is a method of treating cancer in a subject comprising administering to the subject a therapeutically effective amount of a dimer comprising two single chain trimeric CD40L Fc fusion proteins disclosed herein.

In some embodiments, the method further comprises administering a pharmaceutical composition comprising a pharmaceutically acceptable carrier and the single chain trimeric CD40L Fc fusion proteins. In some embodiments, the treatment enhances an innate anti-neoplastic immune response. In some embodiments, the method further comprises co-administration of a second therapy. In some embodiments, said cancer is selected from the group consisting of melanoma, mesothelioma, advanced solid tumor and lymphoma.

In one aspect, provided herein is a method for producing a single chain trimeric CD40L Fc fusion protein or fragment thereof comprising (a) introducing into a host cell a polynucleotide encoding the single chain trimeric CD40L Fc fusion protein disclosed herein; (b) culturing the host cell under conditions to produce the single chain trimeric CD40L Fc fusion protein or fragment thereof, and (c) recovering the single chain trimeric CD40L Fc fusion protein or fragment thereof from the cell or culture. In another aspect, provided herein is a method of producing a dimer comprising two single chain trimeric CD40L Fc fusion proteins, each comprising (a) introducing into a host cell a polynucleotide encoding of the single chain trimeric CD40L Fc fusion protein disclosed herein; (b) culturing the host cell under conditions to produce the single chain trimeric CD40L Fc fusion protein or fragment thereof; (c) recovering the single chain trimeric CD40L Fc fusion protein or fragment thereof from the cell or culture, and (d) combining single chain trimeric CD40L Fc fusion proteins or fragments thereof under conditions that favor dimerization. In yet another aspect, provided herein is a method of producing a pharmaceutical composition of a single chain trimeric CD40L Fc fusion protein or fragment thereof comprising combining the single chain trimeric CD40L Fc fusion protein disclosed herein or fragment thereof with a pharmaceutically acceptable carrier to obtain the pharmaceutical composition.

In one aspect, provided herein is a polypeptide comprising a single chain trimeric CD40L fusion protein, wherein the single chain trimeric CD40L fusion protein comprises three CD40L subunits covalently linked to one another by peptide linkers. In some embodiments, the CD40L subunits comprise a portion of the CD40L extracellular domain. In some embodiments, the CD40L subunits comprise any one of the sequences selected from SEQ ID NOS:20 to 22, or a fragment thereof.

In some embodiments, at least one of the peptide linkers is selected from the group consisting of EGKSSGSGS (SEQ ID NO:23) and (G₃S)₃ (SEQ ID NO:25). In some embodiments, at least two of the peptide linkers are selected from the group consisting of EGKSSGSGS (SEQ ID NO:23) and (G₃S)₃ (SEQ ID NO:25). In some embodiments, at least two of the peptide linkers have the same sequence.

In some embodiments, the single chain trimeric CD40L fusion protein comprises any one sequence selected from SEQ ID NOS:35-38, of a fragment thereof. In some embodiments, the single chain trimeric CD40L fusion protein is fused with a peptide or polypeptide not derived from CD40L.

In another aspect, the single chain trimeric CD40L fusion protein is fused to a peptide tether. In some embodiments, the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28). In some embodiments, the peptide tether is fused to the N-terminus of the single chain trimeric CD40L fusion protein. In some embodiments, the peptide tether is fused to the C-terminus of the single chain trimeric CD40L fusion protein.

In another aspect, the single chain trimeric CD40L fusion protein is fused to an Fc monomer peptide. In some embodiments, the Fc monomer peptide comprises a human Fc sequence. In some embodiments, the human Fc sequence comprises a sequence selected from immunoglobulins IgG, IgA, IgM, IgD and IgE. In some embodiments, the human Fc sequence comprises an IgG sequence. In some embodiments, the IgG sequence is selected from IgG1, IgG2, IgG3 and IgG4. In some embodiments, the IgG sequence comprises an IgG1 sequence. In some embodiments, the IgG1 sequence comprises SEQ ID NOS:30 or 31, or a fragment thereof. In some embodiments, the IgG sequence comprises an IgG2 sequence. In some embodiments, the IgG2 sequence comprises SEQ ID NO:29 or a fragment thereof.

In some embodiments, the single chain trimeric CD40L fusion protein is fused to the Fc monomer peptide via a peptide tether. In some embodiments, the peptide tether comprises between 0 and 20 amino acids. In some embodiments, the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).

In some embodiments, the CD40L trimer is connected to the N-terminus of the Fc monomer peptide. In some embodiments, the single chain trimeric CD40L Fc fusion protein comprises any one sequence selected from SEQ ID NOS:1-12, or a fragment thereof. In some embodiments, the CD40L trimer is connected to the C-terminus of the Fc monomer peptide. In some embodiments, the single chain trimeric CD40L Fc fusion protein comprises any one sequence selected from SEQ ID NOS:13-19, or a fragment thereof.

In another aspect, the single chain trimeric CD40L fusion protein enhances activation of a CD40 polypeptide compared to wild-type CD40L. In some embodiments, the activation of the CD40 polypeptide enhances the immune-stimulatory functions of T cells and/or B cells. In some embodiments, the activation of the CD40 polypeptide comprises enhanced activation of B cells, CD4+ T cells, CD8+ T cells, dendritic cells, macrophages, natural killer cells, monocytes, granulocytes, eosinophils and/or neutrophils compared to wild-type CD40L.

In some embodiments, the activation of the CD40 polypeptide comprises increasing expression of the CD40 polypeptide. In some embodiments, the single chain trimeric CD40L fusion protein enhances anti-tumor activity compared to wild-type CD40L. In some embodiments, the single chain trimeric CD40L fusion protein enhances pro-inflammatory activity compared to wild-type CD40L. In some embodiments, the single chain trimeric CD40L fusion protein enhances clearance of an infectious pathogen compared to wild-type CD40L.

In another aspect, the single chain trimeric CD40L fusion protein increases antibody production by a population of B cells by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In another aspect, the single chain trimeric CD40L fusion protein increases secretion of a pro-inflammatory cytokine by a population of T cells. In some embodiments, the pro-inflammatory cytokine is IL-1, IL-2, IL-6, IL 12, IL-17, IL-22, IL-23, GM-CSF, TNF-α, IFN-γ, or any combination thereof. In some embodiments, the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In another aspect, the single chain trimeric CD40L fusion protein increases a minimal percentage of phagocytotic macrophages in a population of macrophages to about 10%, about 20%, about 30%, about 40%, about 45%, about 50%, about 55%, about 60%, about 65%, about 70%, about 75%, about 80%, about 85%, about 90%, about 95%, or about 99%.

In some embodiments, the single chain trimeric CD40L fusion protein increases a minimal percentage of antigen-presenting dendritic cells in a population of dendritic cells to about 10%, about 20%, about 30%, about 40%, about 45%, about 50%, about 55%, about 60%, about 65%, about 70%, about 75%, about 80%, about 85%, about 90%, about 95%, or about 99%.

In another aspect, the polypeptide is conjugated to an agent. In some embodiments, the agent is selected from the group consisting of a radioisotope, a metal chelator, an enzyme, a fluorescent compound, a bioluminescent compound, and a chemiluminescent compound.

In another aspect, provided herein is a polynucleotide encoding a single chain trimeric CD40L fusion protein, wherein the single chain trimeric CD40L fusion protein comprises three CD40L subunits covalently linked to one another by peptide linkers.

In another aspect, provided herein is a vector comprising a polynucleotide encoding a single chain trimeric CD40L fusion protein, wherein the single chain trimeric CD40L fusion protein comprises three CD40L subunits covalently linked to one another by peptide linkers.

In another aspect, provided herein is a host cell comprising a vector comprising a polynucleotide encoding a single chain trimeric CD40L fusion protein, wherein the single chain trimeric CD40L fusion protein comprises three CD40L subunits covalently linked to one another by peptide linkers.

In another aspect, provided herein is a host cell comprising a polynucleotide encoding a single chain trimeric CD40L fusion protein, wherein the single chain trimeric CD40L fusion protein comprises three CD40L subunits covalently linked to one another by peptide linkers.

In another aspect, provided herein is a pharmaceutical composition comprising a pharmaceutically acceptable carrier and a polypeptide comprising a single chain trimeric CD40L fusion protein, wherein the single chain trimeric CD40L fusion protein comprises three CD40L subunits covalently linked to one another by peptide linkers.

In another aspect, provided herein is a kit comprising a polypeptide comprising a single chain trimeric CD40L fusion protein, wherein the single chain trimeric CD40L fusion protein comprises three CD40L subunits covalently linked to one another by peptide linkers.

In another aspect, provided herein is a system comprising a means for providing a polypeptide comprising a single chain trimeric CD40L fusion protein, wherein the single chain trimeric CD40L fusion protein comprises three CD40L subunits covalently linked to one another by peptide linkers.

In another aspect, provided herein is a method for activating a target cell expressing a CD40 polypeptide comprising contacting the target cell with an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide, wherein said single chain trimeric CD40L fusion protein activates the target cell upon binding the CD40 polypeptide.

In some embodiments, activation of the target cell is measured as increased proliferation or maturation of the target cell. In some embodiments, proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900%, or about 1000%.

In some embodiments, activation of the target cell is measured as prolonged survival time of the target cell. In some embodiments, survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900%, or about 1000%.

In some embodiments, the target cell is antigen presenting cells. In some embodiments, the target cell is natural killer cells, B cells, dendritic cells, macrophages, monocytes, granulocytes, eosinophils, neutrophils, or a combination thereof.

In another aspect, provided herein is a method for promoting antibody production by a population of B cells, comprising contacting the B cells with an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide.

In some embodiments, antibody production by the population of B cells is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900%, or about 1000%.

In another aspect, the B cells are contacted with a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide in the presence of an antigen or a polynucleotide encoding the antigen, and wherein the antibody produced by the B cells specifically binds to the antigen. In some embodiments, the method further promotes formation of memory B cells capable of producing the antibody in response to the antigen.

In some embodiments the polypeptide or the polynucleotide is in a vaccine composition or adjuvant composition. In some embodiments, the antigen or polynucleotide encoding the antigen is in a vaccine composition. In some embodiments, the antigen is originated or derived from an infectious pathogen. In some embodiments, the infectious pathogen is a virus, a bacteria, a fungus, a parasite, or a combination thereof. In some embodiments, the antigen is originated or derived from a diseased cell. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In some embodiments, the antigen is presented by an antigen presenting cell. In some embodiments, the antigen presenting cell is a dendritic cell. In some embodiments, the antigen is associated with an MHC class I or MHC class II complex.

In another aspect, provided herein is a method of increasing secretion of pro-inflammatory cytokines by a population of T cells, comprising contacting the population of T cells with a population of antigen presenting cells in the presence an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide. In some embodiments, the cytokine is IL-1, IL-2, IL-6, IL 12, IL-17, IL-22, IL-23, GM-CSF, TNF-α, IFN-γ, or any combination thereof. In some embodiments, the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900%, or about 1000%.

In another aspect, provided herein is a method of increasing phagocytosis of diseased cells by a population of macrophages, comprising contacting the diseased cells, the macrophages, or both the diseased cells and the macrophage with an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide.

In some embodiments, a minimal percentage of phagocytotic macrophages in the population of macrophages is increased by about 10%, about 20%, about 30%, about 40%, about 45%, about 50%, about 55%, about 60%, about 65%, about 70%, about 75%, about 80%, about 85%, about 90%, about 95%, or about 99%. In some embodiments, the phagocytosis by macrophages is measured by co-culturing macrophages labeled with a first fluorescent dye and diseased cells labeled with a second fluorescent dye, wherein the first fluorescent dye and the second fluorescent dye are different. In some embodiments, the percentage of phagocytotic macrophages is measured by determining the percentage of macrophages comprising the diseased cells. In some embodiments, the diseased cells are cancer cells or cell infected by an infectious pathogen. In some embodiments, the infectious pathogen is a virus, a bacteria, a fungus, a parasite, or a combination thereof.

In another aspect, provided herein is a method of increasing antigen presentation by a population of dendritic cells, comprising contacting the dendritic cells with an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide in the presence of the antigen.

In some embodiments, a minimal percentage of dendritic cells presenting the antigen in the population of dendritic cells is increased by about 10%, about 20%, about 30%, about 40%, about 45%, about 50%, about 55%, about 60%, about 65%, about 70%, about 75%, about 80%, about 85%, about 90%, about 95%, or about 99%. In some embodiments, the antigen presentation by the dendritic cells is measured by co-culturing dendritic cells labeled with a first fluorescent dye and the antigen labeled with a second fluorescent dye, wherein the first fluorescent dye and the second fluorescent dye are different. In some embodiments, percentage of dendritic cells presenting the antigen is measured by determining the percentage of dendritic cells co-localizing with the antigen in the population of dendritic cells.

In some embodiments, the antigen is originated or derived from an infectious pathogen. In some embodiments, the infectious pathogen is a virus, a bacteria, a fungus, a parasite, or a combination thereof. In some embodiments, the antigen is originated or derived from a diseased cell. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In some embodiments, the polypeptide or the polynucleotide is in a vaccine composition or an adjuvant composition. In some embodiments, the antigen or a polynucleotide encoding the antigen is in a vaccine composition.

In another aspect, provided herein is a method of increasing expression of a CD40 polypeptide by a target cell, comprising contacting the target cell with an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide.

In some embodiments, the target cell is a diseased cell. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In some embodiments, the target cell is an immune cell. In some embodiments, the target cell is an antigen presenting cell. In some embodiments, the target cell is natural killer cells, B cells, dendritic cells, macrophages, monocytes, granulocytes, eosinophils, neutrophils, or a combination thereof.

In some embodiments, the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99%.

In another aspect, provided herein is a method of forming a pro-inflammatory milieu in a tissue surrounding a population of diseased cells, comprising contacting the tissue with an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide.

In some embodiments, infiltration of activated B cells, CD4+ T cells, CD8+ T cells, dendritic cells, macrophages, natural killer cells, monocytes, granulocytes, eosinophils and/or neutrophils in the tissue is increased.

In some embodiments, concentration of a pro-inflammatory cytokine is increased in the tissue. In some embodiments, the pro-inflammatory cytokine is IL-1, IL-2, IL-6, IL 12, IL-17, IL-22, IL-23, GM-CSF, TNF-α, IFN-γ, or any combination thereof.

In some embodiments, presentation of antigens originated or derived from the diseased cells by antigen presentation cells is increased in the tissue.

In some embodiments, phagocytosis of the diseased cells is increased in the tissue.

In some embodiments, apoptosis of the diseased cells induced by cell-mediated cytotoxicity is increased in the tissue. In some embodiments, apoptosis of the diseased cells induced by antibody-dependent cellular cytotoxicity is increased in the tissue.

In some embodiments, the population of the diseased cells is reduced in the tissue. In some embodiments, the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99% in the tissue.

In some embodiments, the method is performed in vitro or in vivo.

In another aspect, provided herein is a method of eliminating a diseased cell in a subject, comprising administering an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide.

In some embodiments, the diseased cell does not express a CD40 polypeptide. In some embodiments, the diseased cell expresses a CD40 polypeptide. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In another aspect, provided herein is a method of treating cancer in a subject in need thereof, comprising administering an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide. In some embodiments, the treatment enhances an innate, humoral or cell-mediated anti-neoplastic immune response. In some embodiments, the method further comprises co-administration of a second therapy. In some embodiments, the cancer is selected from the group consisting of melanoma, mesothelioma, advanced solid tumor and lymphoma.

In another aspect, provided herein is a method of treating an infection in a subject in need thereof, comprising administering an effective amount of a polypeptide comprising a single chain trimeric CD40L fusion protein, wherein the single chain trimeric CD40L fusion protein comprises three CD40L subunits covalently linked to one another by peptide linkers or a polynucleotide encoding said polypeptide. In some embodiments, the treatment enhances an innate, humoral, or cell-mediated anti-infective immune response.

In some embodiments, the polypeptide or the polynucleotide is co-administered with a vaccine composition for preventing the infection in the subject. In some embodiments, the polypeptide or the polynucleotide is co-administered with the vaccine composition simultaneously or sequentially.

### 4. BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIGS. 1A-1B show schematic illustrations of examples of trimeric CD40L Fc fusion proteins. FIG. 1A shows an illustration of CD40L trimer design optimization where molecules were designed to address tether length, linker length, and orientation of fusion. FIG. 1B shows an illustration of a “Mono Fc” molecule which is a single chain fusion protein containing a CD40L trimer (circled) fused to a Fc monomer peptide, where the Fc contains mutations in its CH3 domain that abrogate dimer formation of this molecule.

FIGS. 2A-2B show monodispersity of the CD40L Trimer on Fc after protein A purification. FIG. 2A shows SDS-PAGE gel (left panel) and a SEC chromatogram (right panel) of TPP000161222, while FIG. 2B shows SDS-PAGE gel and a SEC chromatogram of TPP000182983.

FIG. 3 shows monodispersity of TPP000182983 after size exclusion chromatography measured by analytical ultra-centrifugation (AUC). All samples were analyzed in duplicate (n=2). The protein runs primarily at ˜7 s with the main species population being 98%. There are also <1% lower molecular weight species (LMWS), <2% dimer, and <1% high molecular weight species (HMWS) present.

FIGS. 4A-4C show binding of CD40 agonists to CD40. FIG. 4A shows binding of CD40L trimer to CD40R measured using Meso Scale Discovery (MSD) bioluminescence assay. FIG. 4B shows binding of TPP000182983 to CD40R measured using and surface plasmon resonance (SPR). FIG. 4C shows binding of TPP000161222 to CD40R measured using and surface plasmon resonance (SPR).

FIGS. 5A-5C show functional activity of the CD40L trimer tested using HEK-Blue reporter assay in FIG. 5A, monocyte derived dendritic cell activation assay in FIG. 5B. and mature dendritic cell activation assay without cross linking antibody treatment in FIG. 5C.

FIGS. 6A-6E show evaluation of functional activity of CD40L Trimer Fc with dendritic cell activation assay using CD86 activation marker (FIG. 6A); CD40 activation marker (FIG. 6B); CD83 activation marker (FIG. 6C); HLA-DR activation marker (FIG. 6D), and PDL1 activation marker (FIG. 6E).

FIGS. 7A-7B show T cell responses generated from moDCs in the presence of CD40 agonists and CEF peptides for CD8+ T cells (FIG. 7A) and CD4+ T cells (FIG. 7B).

### 5. DETAILED DESCRIPTION

The present disclosure is based, in part, on the surprising discovery of single CD40L Fc fusion proteins with favorable biophysical properties and enhanced CD40 activation resulting in enhanced T cell and dendritic cell activation. The compositions and methods of the invention thus provide an avenue for novel and improved therapeutic strategies that target the CD40-CD40L pathway.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series.

Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc. In specific embodiments, the subject is a human. In certain embodiments, the subject is a subject in need thereof.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” (or an equivalent phrase) includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule disclosed herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed molecule can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the terms “antibody” or “immunoglobulin” are used in a broad sense and include human, humanized, composite and chimeric antibodies and antibody fragments that are monoclonal or polyclonal. In general, antibodies are proteins or peptide chains that exhibit binding specificity to a specific antigen. A naturally occurring antibody is a Y shaped molecule that consists of two heavy chains and two light chains folded into constant and variable domains. Antibody structures, including heavy and light chains as well as constant and variable regions within each of the heavy and light chains, are well known. That is, in addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH 3-sheet framework, or one of three hypervariable regions (LCDR1, LCDR2 or LCDR3) within the non-framework region of the antibody VL 3-sheet framework. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4.

The terms “constant region” or “constant domain” refer to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “Fc region” refers to the carboxy terminal portion of an antibody's constant region and encompasses the CH2 and CH3 regions of the heavy chains. The two CH3 domains interact with each other to form a homodimer resulting in dimerization of Fc.

As used herein, the terms “Fc monomer” or “mFc” or “Fc monomer peptide” refer to a molecule that includes the carboxy terminal portion of a monomeric Fc region that consists of the CH2 and CH3 regions of the heavy chain, or a portion thereof. In some embodiments, the Fc monomer peptide comprises a human Fc sequence. In some embodiments, the human Fc sequence comprises a sequence selected from immunoglobulins IgG, IgA, IgM, IgD and IgE. In one embodiment, the human Fc sequence comprises an IgG sequence. In some embodiments, the IgG sequence is selected from IgG1, IgG2, IgG3 and IgG4. In some embodiments, the IgG sequence is an IgG1 sequence. In some embodiments, the IgG1 sequence comprises SEQ ID NOS:30 or 31, or a fragment thereof. In one embodiment, the IgG1 sequence comprises SEQ ID NO:30 or a fragment thereof. In another embodiment, the IgG1 sequence comprises SEQ ID NO:31 or a fragment thereof. In some embodiments, the IgG sequence comprises an IgG2 sequence. In some embodiments, the IgG2 sequence comprises SEQ ID NO:29 or a fragment thereof.

The single chain trimeric CD40L Fc fusion proteins provided herein can comprise Fc monomer peptides of any of the five major classes or corresponding sub-classes. In specific embodiments, the Fc monomer peptides provided herein are IgG1, IgG2, IgG3 or IgG4. In further embodiments, the Fc monomer peptides provided herein are human IgG1 and human IgG2 isotypes. In additional embodiments, the Fc monomer peptide component of a single chain trimeric CD40L Fc fusion protein has silenced effector functions.

Accordingly, the single chain trimeric CD40L Fc fusion proteins provided herein can contain an Fc monomer peptide corresponding to a kappa or lambda light chain constant domain. According to particular embodiments, the single chain trimeric CD40L Fc fusion proteins disclosed herein include Fc monomers with heavy and/or light chain constant regions from rat or human antibodies.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies disclosed herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene. Accordingly, the single chain trimeric CD40L Fc fusion proteins provided herein can comprise Fc monomers from a monoclonal antibody.

As used herein, the term “single-chain antibody” refers to a conventional single-chain antibody in the field, which comprises a heavy chain variable region and a light chain variable region connected by a short peptide of about 15 to about 20 amino acids. Accordingly, the single chain trimeric CD40L Fc fusion proteins provided herein can comprise Fc monomers from a single-chain antibody.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide. Accordingly, the single chain trimeric CD40L Fc fusion proteins provided herein can comprise Fc monomers from a human antibody.

As used herein, the term “humanized antibody” refers to a non-human antibody that is modified to increase the sequence homology to that of a human antibody, such that the antigen-binding properties of the antibody are retained, but its antigenicity in the human body is reduced. Accordingly, the single chain trimeric CD40L Fc fusion proteins provided herein can comprise Fc monomers from a humanized antibody.

As used herein, the terms “CD40 ligand” or “CD40L” refer to a protein, or fragment of a protein, that acts as a ligand to CD40/TNFRSF5, a costimulatory member of the tumor necrosis factor receptor (TNFR) superfamily. Particular CD40L sequences, can be obtained from publicly accessible databases such as UniProt, for example, at UniProt Accession No. P29965. CD40L consists of an extracellular domain, stalk region, transmembrane helix, and short cytoplasmic domain. The activity of CD40L is located to the 215 amino acid extracellular domain, which is characterized by a sandwich structure composed of a β-sheet, α-helix loop, and a β-sheet. This structure allows for the trimerization of CD40L. The term CD40L further encompasses polypeptides or any fragments thereof having at least about having at least about 50, at least 55, at least 60, at least 65, at least 70, at least 75, at least 80, at least 85, at least 90, at least 95, at least 98 or at least 99%, sequence identity and ability to act as a ligand to CD40. The term CD40L encompasses full length CD40L and any fragments thereof, e.g., extracellular domain portions of CD40L. The term further encompasses soluble CD40L (sCD40L), which has been reported in different disease settings and can exist in monomeric and multimeric forms. In the context of a single chain trimeric CD40L fusion protein or a single chain trimeric CD40L Fc fusion protein according to the present disclosure, the individual CD40L components are referred to as “CD40L subunits.” The portion of the fusion protein consisting of the three CD40L subunits fused together can also be referred to as a “CD40L trimer.”

As used herein, the term “fusion protein” refers to a protein or a polypeptide that encompasses two or more peptide segments linked together to create a sequence that is not present in the same naturally occurring polypeptide.

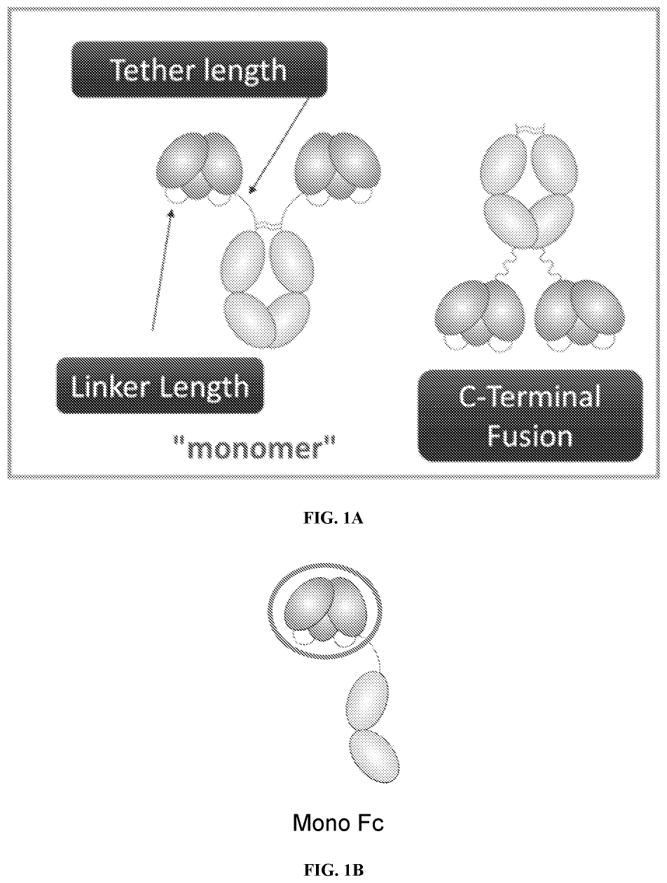

As used herein, the term “single chain trimeric CD40L Fc fusion protein” refers to a protein comprising a monomeric Fc region (mFc), generally linked (optionally through a peptide tether, as described herein) to three CD40L subunits, as described herein and shown in FIG. 1. In particular, a single chain trimeric CD40L Fc fusion protein consists of three CD40L extracellular domains (ECD), or fragments thereof, connected to each other with flexible peptide linkers to form a linear CD40L trimer that is connected with a peptide tether to either the N-terminus or the C-terminus of an Fc monomer peptide. Accordingly, in different embodiments, a single chain trimeric CD40L Fc fusion protein can be either a C-terminal or an N-terminal Fc fusion of the CD40L trimer.

In some embodiments, the CD40L trimer is connected to the N-terminus of the Fc monomer peptide. In some embodiments, the single chain trimeric CD40L Fc fusion protein comprises any one sequence selected from SEQ ID NOS:1-12, or a fragment thereof.

In one embodiment, the single chain trimeric CD40L Fc fusion protein comprises SEQ ID NO:1. In another embodiment, the single chain trimeric CD40L Fc fusion protein comprises SEQ ID NO:2. In another embodiment, the single chain trimeric CD40L Fc fusion protein comprises SEQ ID NO:3. In another embodiment, the single chain trimeric CD40L Fc fusion protein comprises SEQ ID NO:4. In another embodiment, the single chain trimeric CD40L Fc fusion protein comprises SEQ ID NO:5. In another embodiment, the single chain trimeric CD40L Fc fusion protein comprises SEQ ID NO:6. In another embodiment, the single chain trimeric CD40L Fc fusion protein comprises SEQ ID NO:7. In another embodiment, the single chain trimeric CD40L Fc fusion protein comprises SEQ ID NO:8. In another embodiment, the single chain trimeric CD40L Fc fusion protein comprises SEQ ID NO:9. In another embodiment, the single chain trimeric CD40L Fc fusion protein comprises SEQ ID NO:10. In another embodiment, the single chain trimeric CD40L Fc fusion protein comprises SEQ ID NO:11. In another embodiment, the single chain trimeric CD40L Fc fusion protein comprises SEQ ID NO:12. In one embodiment, the single chain trimeric CD40L Fc fusion protein comprises a fragment of SEQ ID NO:1. In another embodiment, the single chain trimeric CD40L Fc fusion protein comprises a fragment of SEQ ID NO:2. In another embodiment, the single chain trimeric CD40L Fc fusion protein comprises a fragment of SEQ ID NO:3. In another embodiment, the single chain trimeric CD40L Fc fusion protein comprises a fragment of SEQ ID NO:4. In another embodiment, the single chain trimeric CD40L Fc fusion protein comprises a fragment of SEQ ID NO:5. In another embodiment, the single chain trimeric CD40L Fc fusion protein comprises a fragment of SEQ ID NO:6. In another embodiment, the single chain trimeric CD40L Fc fusion protein comprises a fragment of SEQ ID NO:7. In another embodiment, the single chain trimeric CD40L Fc fusion protein comprises a fragment of SEQ ID NO:8. In another embodiment, the single chain trimeric CD40L Fc fusion protein comprises a fragment of SEQ ID NO:9. In another embodiment, the single chain trimeric CD40L Fc fusion protein comprises a fragment of SEQ ID NO:10. In another embodiment, the single chain trimeric CD40L Fc fusion protein comprises a fragment of SEQ ID NO:11. In another embodiment, the single chain trimeric CD40L Fc fusion protein comprises a fragment of SEQ ID NO:12.

In some embodiments, the CD40L trimer is connected to the C-terminus of the Fc monomer peptide. In some embodiments, the single chain trimeric CD40L Fc fusion protein comprises any one sequence selected from SEQ ID NOS:13-19, or a fragment thereof. In one embodiment, the single chain trimeric CD40L Fc fusion protein comprises SEQ ID NO:16 or a fragment thereof.

In one embodiment, the single chain trimeric CD40L Fc fusion protein comprises SEQ ID NO:13. In another embodiment, the single chain trimeric CD40L Fc fusion protein comprises SEQ ID NO:14. In another embodiment, the single chain trimeric CD40L Fc fusion protein comprises SEQ ID NO:15. In another embodiment, the single chain trimeric CD40L Fc fusion protein comprises SEQ ID NO:16. In another embodiment, the single chain trimeric CD40L Fc fusion protein comprises SEQ ID NO:17. In another embodiment, the single chain trimeric CD40L Fc fusion protein comprises SEQ ID NO:18. In another embodiment, the single chain trimeric CD40L Fc fusion protein comprises SEQ ID NO:19.

In one embodiment, the single chain trimeric CD40L Fc fusion protein comprises a fragment of SEQ ID NO:13. In another embodiment, the single chain trimeric CD40L Fc fusion protein comprises a fragment of SEQ ID NO:14. In another embodiment, the single chain trimeric CD40L Fc fusion protein comprises a fragment of SEQ ID NO:15. In another embodiment, the single chain trimeric CD40L Fc fusion protein comprises a fragment of SEQ ID NO:16. In another embodiment, the single chain trimeric CD40L Fc fusion protein comprises a fragment of SEQ ID NO:17. In another embodiment, the single chain trimeric CD40L Fc fusion protein comprises a fragment of SEQ ID NO:18. In another embodiment, the single chain trimeric CD40L Fc fusion protein comprises a fragment of SEQ ID NO:19.

In some embodiments, the CD40 ligand subunits comprise a portion of the CD40L extracellular sequence. In some embodiments, the CD40L subunits comprise any one of the sequences selected from SEQ ID NOS:20-22, or a fragment thereof.

In one embodiment, the CD40L subunits comprise a fragment of SEQ ID NO:20. In another embodiment, the CD40L subunits comprise a fragment of SEQ ID NO:21. In another embodiment, the CD40L subunits comprise a fragment of SEQ ID NO:22.

In one embodiment, the CD40L subunits comprise SEQ ID NO:20. In another embodiment, the CD40L subunits comprise SEQ ID NO:21. In another embodiment, the CD40L subunits comprise SEQ ID NO:22.

In some instances, two single chain trimeric CD40L Fc fusion proteins can form a homodimeric Fc fusion protein or a heterodimeric Fc fusion protein with the latter being preferred. In one embodiment, two single chain trimeric CD40L Fc fusion proteins form a homodimeric Fc fusion protein. In one embodiment, two single chain trimeric CD40L Fc fusion proteins form a heterodimeric Fc fusion protein. In some cases, one monomer of the heterodimeric Fc fusion protein comprises only an Fc monomer peptide or fragment thereof and the other monomer is a single chain trimeric CD40L Fc fusion protein. In some embodiments, single chain trimeric CD40L Fc fusion proteins can include a variant Fc monomer with one or more amino acid substitutions compared to a reference or wild-type Fc monomer. In some cases, one monomer of the heterodimeric Fc fusion protein an Fc monomer peptide or fragment thereof and a protein domain other than CD40L, such as a receptor, ligand or other binding partner. Accordingly, a single chain trimeric CD40L Fc fusion protein can be a component of a heterodimeric Fc fusion protein that is a bispecific molecule.

The term “bispecific molecule” refers to a molecule that has two binding domains, each capable of specifically binding a target protein, ligand or fragments thereof. As such, while a bispecific molecule can include binding domains that are non-antibody proteins, ligands and fragments thereof, including recombinant antigens, that can specifically bind another protein. In an embodiment, a bispecific molecule can comprise a single chain trimeric CD40L Fc fusion protein.

The terms “binds” or “binding” refer to an interaction between molecules including, for example, to form a complex. The term “binding domain” refers to a portion of a molecule responsible for a specific binding interaction with another molecule or ligand. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the binding molecule. The value of K_{D} varies for different complexes of binding molecules and their ligands (i.e. antibody and antigen) and depends on both kₒₙ and k_{off}. A binding domain that can specifically bind the target with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less. The dissociation constant K_{D} for a binding molecule provided herein can be determined using any method provided herein or any other method well known to those skilled in the art. The affinity at one binding site does not always reflect the true strength of the interaction between binding molecule and its binding partner.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen may be cross-reactive with related antigens. In certain embodiments, a binding molecule or antigen binding domain that binds to or specifically binds to an antigen does not cross-react with other antigens. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. A binding molecule or antigen binding domain that binds to an antigen includes one that is capable of binding the antigen with sufficient affinity such that the binding molecule is useful, for example, as a diagnostic agent in targeting the antigen. In certain embodiments, a binding molecule or antigen binding domain that binds to an antigen has an equilibrium dissociation constant (K_{D}) of less than or equal to 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as CD40L) and its binding partner (e.g., CD40/TNFRSF5). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the equilibrium dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

In one aspect, provided herein is a polypeptide comprising a single chain trimeric CD40L fusion protein. In some embodiments, the single chain trimeric CD40L fusion protein comprises three CD40L subunits covalently linked to one another by flexible peptide linkers. In some embodiments, the single chain trimeric CD40L fusion protein comprises three CD40L ECD domains, or fragments thereof. In particular embodiments, each CD40L subunit of the single chain trimeric CD40L fusion protein comprises any one sequence selected from:

| | |
|---|---|
| CD40L v.1 | NPQIAAHVISEASSKTTSVLQWAEKGYYTMSNNLVTLENGKQLTV |
| (SEQ ID NO: 20) | KRQGLYYIYAQVTFCSNREASSQAPFIASLWLKSPGRFERILLRAA |
| | NTHSSAKPCGQQSIHLGGVFELQPGASVFVNVTDPSQVSHGTGFTS |
| | FGLLKL |
| CD40L v.2 | QIAAHVISEASSKTTSVLQWAEKGYYTMSNNLVTLENGKQLTVK |
| (SEQ ID NO: 21) | RQGLYYIYAQVTFCSNREASSQAPFIASLWLKSPGRFERILLRAAN |
| | THSSAKPCGQQSIHLGGVFELQPGASVFVNVTDPSQVSHGTGFTSF |
| | GLLKL |
| CD40L v.3 | NPQIAAHVISEASSKTTSVLQWAEKGYYTMSNNLVTLENGKQLTV |
| (SEQ ID NO: 22) | KRQGLYYIYAQVTFCSNREASSQAPFIASLCLKSPGRFERILLRAAN |
| | THSSAKPCGQQSIHLGGVFELQPGASVFVNVTDPSQVSHGTGFTSF |
| | GLLKL |
| CD40L v.4 | IAAHVISEASSKTTSVLQWAEKGYYTMSNNLVTLENGKQLTVKR |
| (SEQ ID NO: 33) | QGLYYIYAQVTFCSNREASSQAPFIASLCLKSPGRFERILLRAANTH |
| | SSAKPCGQQSIHLGGVFELQPGASVFVNVTDPSQVSHGTGFTSFGL |
| | LKL |
| CD40L v.5 | QIAAHVISEASSKTTSVLQWAEKGYYTMSNNLVTLENGKQLTVK |
| (SEQ ID NO: 34) | RQGLYYIYAQVTFCSNREASSQAPFIASLCLKSPGRFERILLRAANT |
| | HSSAKPCGQQSIHLGGVFELQPGASVFVNVTDPSQVSHGTGFTSFG |
| | LLKL |

In one embodiment, a CD40L subunit of the single chain trimeric CD40L fusion protein comprises SEQ ID NO:20 or a fragment thereof. In another embodiment, a CD40L subunit of the single chain trimeric CD40L fusion protein comprises SEQ ID NO:21 or a fragment thereof. In another embodiment, a CD40L subunit of the single chain trimeric CD40L fusion protein comprises SEQ ID NO:22 or a fragment thereof. In another embodiment, a CD40L subunit of the single chain trimeric CD40L fusion protein comprises SEQ ID NO:33 or a fragment thereof. In another embodiment, a CD40L subunit of the single chain trimeric CD40L fusion protein comprises SEQ TD NO:34 or a fragment thereof. In one embodiment, a CD40L subunit of the single chain trimeric CD40L fusion protein comprises SEQ ID NO:20. In another embodiment, a CD40L subunit of the single chain trimeric CD40L fusion protein comprises SEQ ID NO:21. In another embodiment, a CD40L subunit of the single chain trimeric CD40L fusion protein comprises SEQ ID NO:22. In another embodiment, a CD40L subunit of the single chain trimeric CD40L fusion protein comprises SEQ ID NO:33. In another embodiment, a CD40L subunit of the single chain trimeric CD40L fusion protein comprises SEQ ID NO:34. In one embodiment, a CD40L subunit of the single chain trimeric CD40L fusion protein comprises a fragment of SEQ ID NO:20. In another embodiment, a CD40L subunit of the single chain trimeric CD40L fusion protein comprises a fragment of SEQ ID NO:21. In another embodiment, a CD40L subunit of the single chain trimeric CD40L fusion protein comprises a fragment of SEQ ID NO:22. In another embodiment, a CD40L subunit of the single chain trimeric CD40L fusion protein comprises a fragment of SEQ ID NO:33. In another embodiment, a CD40L subunit of the single chain trimeric CD40L fusion protein comprises a fragment of SEQ ID NO:34.

In one embodiment, a single chain trimeric CD40L fusion protein comprises a CD40L subunit having the amino acid sequence of SEQ ID NO:20. In one embodiment, a single chain trimeric CD40L fusion protein comprises a CD40L subunit having the amino acid sequence of SEQ ID NO:21. In one embodiment, a single chain trimeric CD40L fusion protein comprises a CD40L subunit having the amino acid sequence of SEQ ID NO:22. In one embodiment, a single chain trimeric CD40L fusion protein comprises a CD40L subunit having the amino acid sequence of SEQ ID NO:33. In one embodiment, a single chain trimeric CD40L fusion protein comprises a CD40L subunit having the amino acid sequence of SEQ ID NO:34.

In one embodiment, a single chain trimeric CD40L fusion protein comprises a first CD40L subunit having the amino acid sequence of SEQ ID NO:20, and a second CD40L subunit having the amino acid sequence of SEQ ID NO:21. In one embodiment, a single chain trimeric CD40L fusion protein comprises a first CD40L subunit having the amino acid sequence of SEQ ID NO:20, and a second CD40L subunit having the amino acid sequence of SEQ ID NO:22. In one embodiment, a single chain trimeric CD40L fusion protein comprises a first CD40L subunit having the amino acid sequence of SEQ ID NO:20, and a second CD40L subunit having the amino acid sequence of SEQ ID NO:33. In one embodiment, a single chain trimeric CD40L fusion protein comprises a first CD40L subunit having the amino acid sequence of SEQ ID NO:20, and a second CD40L subunit having the amino acid sequence of SEQ ID NO:34. In one embodiment, a single chain trimeric CD40L fusion protein comprises a first CD40L subunit having the amino acid sequence of SEQ ID NO:21, and a second CD40L subunit having the amino acid sequence of SEQ ID NO:22. In one embodiment, a single chain trimeric CD40L fusion protein comprises a first CD40L subunit having the amino acid sequence of SEQ ID NO:21, and a second CD40L subunit having the amino acid sequence of SEQ ID NO:33. In one embodiment, a single chain trimeric CD40L fusion protein comprises a first CD40L subunit having the amino acid sequence of SEQ ID NO:21, and a second CD40L subunit having the amino acid sequence of SEQ ID NO:34. In one embodiment, a single chain trimeric CD40L fusion protein comprises a first CD40L subunit having the amino acid sequence of SEQ ID NO:22, and a second CD40L subunit having the amino acid sequence of SEQ ID NO:33. In one embodiment, a single chain trimeric CD40L fusion protein comprises a first CD40L subunit having the amino acid sequence of SEQ ID NO:22, and a second CD40L subunit having the amino acid sequence of SEQ ID NO:34. In one embodiment, a single chain trimeric CD40L fusion protein comprises a first CD40L subunit having the amino acid sequence of SEQ ID NO:33, and a second CD40L subunit having the amino acid sequence of SEQ ID NO:34.

In one embodiment, a single chain trimeric CD40L fusion protein comprises a first CD40L subunit having the amino acid sequence of SEQ ID NO:20, a second CD40L subunit having the amino acid sequence of SEQ ID NO:20, and a third CD40L subunit having the amino acid sequence of SEQ ID NO:20. In one embodiment, a single chain trimeric CD40L fusion protein comprises a first CD40L subunit having the amino acid sequence of SEQ ID NO:20, a second CD40L subunit having the amino acid sequence of SEQ ID NO:21, and a third CD40L subunit having the amino acid sequence of SEQ ID NO:20. In one embodiment, a single chain trimeric CD40L fusion protein comprises a first CD40L subunit having the amino acid sequence of SEQ ID NO:20, a second CD40L subunit having the amino acid sequence of SEQ ID NO:22, and a third CD40L subunit having the amino acid sequence of SEQ ID NO:20. In one embodiment, a single chain trimeric CD40L fusion protein comprises a first CD40L subunit having the amino acid sequence of SEQ ID NO:20, a second CD40L subunit having the amino acid sequence of SEQ ID NO:33, and a third CD40L subunit having the amino acid sequence of SEQ ID NO:20. In one embodiment, a single chain trimeric CD40L fusion protein comprises a first CD40L subunit having the amino acid sequence of SEQ ID NO:20, a second CD40L subunit having the amino acid sequence of SEQ ID NO:34, and a third CD40L subunit having the amino acid sequence of SEQ ID NO:20. In one embodiment, a single chain trimeric CD40L fusion protein comprises a first CD40L subunit having the amino acid sequence of SEQ ID NO:21, a second CD40L subunit having the amino acid sequence of SEQ ID NO:20, and a third CD40L subunit having the amino acid sequence of SEQ ID NO:20. In one embodiment, a single chain trimeric CD40L fusion protein comprises a first CD40L subunit having the amino acid sequence of SEQ ID NO:21, a second CD40L subunit having the amino acid sequence of SEQ ID NO:21, and a third CD40L subunit having the amino acid sequence of SEQ ID NO:20. In one embodiment, a single chain trimeric CD40L fusion protein comprises a first CD40L subunit having the amino acid sequence of SEQ ID NO:21, a second CD40L subunit having the amino acid sequence of SEQ ID NO:22, and a third CD40L subunit having the amino acid sequence of SEQ ID NO:20. In one embodiment, a single chain trimeric CD40L fusion protein comprises a first CD40L subunit having the amino acid sequence of SEQ ID NO:21, a second CD40L subunit having the amino acid sequence of SEQ ID NO:33, and a third CD40L subunit having the amino acid sequence of SEQ ID NO:20. In one embodiment, a single chain trimeric CD40L fusion protein comprises a first CD40L subunit having the amino acid sequence of SEQ ID NO:21, a second CD40L subunit having the amino acid sequence of SEQ ID NO:34, and a third CD40L subunit having the amino acid sequence of SEQ ID NO:20.

In one embodiment, a single chain trimeric CD40L fusion protein comprises a first CD40L subunit having the amino acid sequence of SEQ ID NO:22, a second CD40L subunit having the amino acid sequence of SEQ ID NO:20, and a third CD40L subunit having the amino acid sequence of SEQ ID NO:20. In one embodiment, a single chain trimeric CD40L fusion protein comprises a first CD40L subunit having the amino acid sequence of SEQ ID NO:22, a second CD40L subunit having the amino acid sequence of SEQ ID NO:21, and a third CD40L subunit having the amino acid sequence of SEQ ID NO:20. In one embodiment, a single chain trimeric CD40L fusion protein comprises a first CD40L subunit having the amino acid sequence of SEQ ID NO:22, a second CD40L subunit having the amino acid sequence of SEQ ID NO:22, and a third CD40L subunit having the amino acid sequence of SEQ ID NO:20. In one embodiment, a single chain trimeric CD40L fusion protein comprises a first CD40L subunit having the amino acid sequence of SEQ ID NO:22, a second CD40L subunit having the amino acid sequence of SEQ ID NO:33, and a third CD40L subunit having the amino acid sequence of SEQ ID NO:20. In one embodiment, a single chain trimeric CD40L fusion protein comprises a first CD40L subunit having the amino acid sequence of SEQ ID NO:22, a second CD40L subunit having the amino acid sequence of SEQ ID NO:34, and a third CD40L subunit having the amino acid sequence of SEQ ID NO:20.

In one embodiment, a single chain trimeric CD40L fusion protein comprises a first CD40L subunit having the amino acid sequence of SEQ ID NO:33, a second CD40L subunit having the amino acid sequence of SEQ ID NO:20, and a third CD40L subunit having the amino acid sequence of SEQ ID NO:20. In one embodiment, a single chain trimeric CD40L fusion protein comprises a first CD40L subunit having the amino acid sequence of SEQ ID NO:33, a second CD40L subunit having the amino acid sequence of SEQ ID NO:21, and a third CD40L subunit having the amino acid sequence of SEQ ID NO:20. In one embodiment, a single chain trimeric CD40L fusion protein comprises a first CD40L subunit having the amino acid sequence of SEQ ID NO:33, a second CD40L subunit having the amino acid sequence of SEQ ID NO:22, and a third CD40L subunit having the amino acid sequence of SEQ ID NO:20. In one embodiment, a single chain trimeric CD40L fusion protein comprises a first CD40L subunit having the amino acid sequence of SEQ ID NO:33, a second CD40L subunit having the amino acid sequence of SEQ ID NO:33, and a third CD40L subunit having the amino acid sequence of SEQ ID NO:20. In one embodiment, a single chain trimeric CD40L fusion protein comprises a first CD40L subunit having the amino acid sequence of SEQ ID NO:33, a second CD40L subunit having the amino acid sequence of SEQ ID NO:34, and a third CD40L subunit having the amino acid sequence of SEQ ID NO:20. In one embodiment, a single chain trimeric CD40L fusion protein comprises a first CD40L subunit having the amino acid sequence of SEQ ID NO:34, a second CD40L subunit having the amino acid sequence of SEQ ID NO:20, and a third CD40L subunit having the amino acid sequence of SEQ ID NO:20. In one embodiment, a single chain trimeric CD40L fusion protein comprises a first CD40L subunit having the amino acid sequence of SEQ ID NO:34, a second CD40L subunit having the amino acid sequence of SEQ ID NO:21, and a third CD40L subunit having the amino acid sequence of SEQ ID NO:20. In one embodiment, a single chain trimeric CD40L fusion protein comprises a first CD40L subunit having the amino acid sequence of SEQ ID NO:34, a second CD40L subunit having the amino acid sequence of SEQ ID NO:22, and a third CD40L subunit having the amino acid sequence of SEQ ID NO:20. In one embodiment, a single chain trimeric CD40L fusion protein comprises a first CD40L subunit having the amino acid sequence of SEQ ID NO:34, a second CD40L subunit having the amino acid sequence of SEQ ID NO:33, and a third CD40L subunit having the amino acid sequence of SEQ ID NO:20. In one embodiment, a single chain trimeric CD40L fusion protein comprises a first CD40L subunit having the amino acid sequence of SEQ ID NO:34, a second CD40L subunit having the amino acid sequence of SEQ ID NO:34, and a third CD40L subunit having the amino acid sequence of SEQ ID NO:20.

In one embodiment, a single chain trimeric CD40L fusion protein comprises a first CD40L subunit having the amino acid sequence of SEQ ID NO:20, a second CD40L subunit having the amino acid sequence of SEQ ID NO:20, and a third CD40L subunit having the amino acid sequence of SEQ ID NO:21. In one embodiment, a single chain trimeric CD40L fusion protein comprises a first CD40L subunit having the amino acid sequence of SEQ ID NO:20, a second CD40L subunit having the amino acid sequence of SEQ ID NO:21, and a third CD40L subunit having the amino acid sequence of SEQ ID NO:21. In one embodiment, a single chain trimeric CD40L fusion protein comprises a first CD40L subunit having the amino acid sequence of SEQ ID NO:20, a second CD40L subunit having the amino acid sequence of SEQ ID NO:22, and a third CD40L subunit having the amino acid sequence of SEQ ID NO:21. In one embodiment, a single chain trimeric CD40L fusion protein comprises a first CD40L subunit having the amino acid sequence of SEQ ID NO:20, a second CD40L subunit having the amino acid sequence of SEQ ID NO:33, and a third CD40L subunit having the amino acid sequence of SEQ ID NO:21. In one embodiment, a single chain trimeric CD40L fusion protein comprises a first CD40L subunit having the amino acid sequence of SEQ ID NO:20, a second CD40L subunit having the amino acid sequence of SEQ ID NO:34, and a third CD40L subunit having the amino acid sequence of SEQ ID NO:21. In one embodiment, a single chain trimeric CD40L fusion protein comprises a first CD40L subunit having the amino acid sequence of SEQ ID NO:21, a second CD40L subunit having the amino acid sequence of SEQ ID NO:20, and a third CD40L subunit having the amino acid sequence of SEQ ID NO:21. In one embodiment, a single chain trimeric CD40L fusion protein comprises a first CD40L subunit having the amino acid sequence of SEQ ID NO:21, a second CD40L subunit having the amino acid sequence of SEQ ID NO:21, and a third CD40L subunit having the amino acid sequence of SEQ ID NO:21. In one embodiment, a single chain trimeric CD40L fusion protein comprises a first CD40L subunit having the amino acid sequence of SEQ ID NO:21, a second CD40L subunit having the amino acid sequence of SEQ ID NO:22, and a third CD40L subunit having the amino acid sequence of SEQ ID NO:21. In one embodiment, a single chain trimeric CD40L fusion protein comprises a first CD40L subunit having the amino acid sequence of SEQ ID NO:21, a second CD40L subunit having the amino acid sequence of SEQ ID NO:33, and a third CD40L subunit having the amino acid sequence of SEQ ID NO:21. In one embodiment, a single chain trimeric CD40L fusion protein comprises a first CD40L subunit having the amino acid sequence of SEQ ID NO:21, a second CD40L subunit having the amino acid sequence of SEQ ID NO:34, and a third CD40L subunit having the amino acid sequence of SEQ ID NO:21. In one embodiment, a single chain trimeric CD40L fusion protein comprises a first CD40L subunit having the amino acid sequence of SEQ ID NO:22, a second CD40L subunit having the amino acid sequence of SEQ ID NO:20, and a third CD40L subunit having the amino acid sequence of SEQ ID NO:21. In one embodiment, a single chain trimeric CD40L fusion protein comprises a first CD40L subunit having the amino acid sequence of SEQ ID NO:22, a second CD40L subunit having the amino acid sequence of SEQ ID NO:21, and a third CD40L subunit having the amino acid sequence of SEQ ID NO:21. In one embodiment, a single chain trimeric CD40L fusion protein comprises a first CD40L subunit having the amino acid sequence of SEQ ID NO:22, a second CD40L subunit having the amino acid sequence of SEQ ID NO:22, and a third CD40L subunit having the amino acid sequence of SEQ ID NO:21. In one embodiment, a single chain trimeric CD40L fusion protein comprises a first CD40L subunit having the amino acid sequence of SEQ ID NO:22, a second CD40L subunit having the amino acid sequence of SEQ ID NO:33, and a third CD40L subunit having the amino acid sequence of SEQ ID NO:21. In one embodiment, a single chain trimeric CD40L fusion protein comprises a first CD40L subunit having the amino acid sequence of SEQ ID NO:22, a second CD40L subunit having the amino acid sequence of SEQ ID NO:34, and a third CD40L subunit having the amino acid sequence of SEQ ID NO:21. In one embodiment, a single chain trimeric CD40L fusion protein comprises a first CD40L subunit having the amino acid sequence of SEQ ID NO:33, a second CD40L subunit having the amino acid sequence of SEQ ID NO:20, and a third CD40L subunit having the amino acid sequence of SEQ ID NO:21. In one embodiment, a single chain trimeric CD40L fusion protein comprises a first CD40L subunit having the amino acid sequence of SEQ ID NO:33, a second CD40L subunit having the amino acid sequence of SEQ ID NO:21, and a third CD40L subunit having the amino acid sequence of SEQ ID NO:21. In one embodiment, a single chain trimeric CD40L fusion protein comprises a first CD40L subunit having the amino acid sequence of SEQ ID NO:33, a second CD40L subunit having the amino acid sequence of SEQ ID NO:22, and a third CD40L subunit having the amino acid sequence of SEQ ID NO:21. In one embodiment, a single chain trimeric CD40L fusion protein comprises a first CD40L subunit having the amino acid sequence of SEQ ID NO:33, a second CD40L subunit having the amino acid sequence of SEQ ID NO:33, and a third CD40L subunit having the amino acid sequence of SEQ ID NO:21. In one embodiment, a single chain trimeric CD40L fusion protein comprises a first CD40L subunit having the amino acid sequence of SEQ ID NO:33, a second CD40L subunit having the amino acid sequence of SEQ ID NO:34, and a third CD40L subunit having the amino acid sequence of SEQ ID NO:21. In one embodiment, a single chain trimeric CD40L fusion protein comprises a first CD40L subunit having the amino acid sequence of SEQ ID NO:34, a second CD40L subunit having the amino acid sequence of SEQ ID NO:20, and a third CD40L subunit having the amino acid sequence of SEQ ID NO:21. In one embodiment, a single chain trimeric CD40L fusion protein comprises a first CD40L subunit having the amino acid sequence of SEQ ID NO:34, a second CD40L subunit having the amino acid sequence of SEQ ID NO:21, and a third CD40L subunit having the amino acid sequence of SEQ ID NO:21. In one embodiment, a single chain trimeric CD40L fusion protein comprises a first CD40L subunit having the amino acid sequence of SEQ ID NO:34, a second CD40L subunit having the amino acid sequence of SEQ ID NO:22, and a third CD40L subunit having the amino acid sequence of SEQ ID NO:21. In one embodiment, a single chain trimeric CD40L fusion protein comprises a first CD40L subunit having the amino acid sequence of SEQ ID NO:34, a second CD40L subunit having the amino acid sequence of SEQ ID NO:33, and a third CD40L subunit having the amino acid sequence of SEQ ID NO:21. In one embodiment, a single chain trimeric CD40L fusion protein comprises a first CD40L subunit having the amino acid sequence of SEQ ID NO:34, a second CD40L subunit having the amino acid sequence of SEQ ID NO:34, and a third CD40L subunit having the amino acid sequence of SEQ ID NO:21.

In one embodiment, a single chain trimeric CD40L fusion protein comprises a first CD40L subunit having the amino acid sequence of SEQ ID NO:20, a second CD40L subunit having the amino acid sequence of SEQ ID NO:20, and a third CD40L subunit having the amino acid sequence of SEQ ID NO:22. In one embodiment, a single chain trimeric CD40L fusion protein comprises a first CD40L subunit having the amino acid sequence of SEQ ID NO:20, a second CD40L subunit having the amino acid sequence of SEQ ID NO:21, and a third CD40L subunit having the amino acid sequence of SEQ ID NO:22. In one embodiment, a single chain trimeric CD40L fusion protein comprises a first CD40L subunit having the amino acid sequence of SEQ ID NO:20, a second CD40L subunit having the amino acid sequence of SEQ ID NO:22, and a third CD40L subunit having the amino acid sequence of SEQ ID NO:22. In one embodiment, a single chain trimeric CD40L fusion protein comprises a first CD40L subunit having the amino acid sequence of SEQ ID NO:20, a second CD40L subunit having the amino acid sequence of SEQ ID NO:33, and a third CD40L subunit having the amino acid sequence of SEQ ID NO:22. In one embodiment, a single chain trimeric CD40L fusion protein comprises a first CD40L subunit having the amino acid sequence of SEQ ID NO:20, a second CD40L subunit having the amino acid sequence of SEQ ID NO:34, and a third CD40L subunit having the amino acid sequence of SEQ ID NO:22. In one embodiment, a single chain trimeric CD40L fusion protein comprises a first CD40L subunit having the amino acid sequence of SEQ ID NO:21, a second CD40L subunit having the amino acid sequence of SEQ ID NO:20, and a third CD40L subunit having the amino acid sequence of SEQ ID NO:22. In one embodiment, a single chain trimeric CD40L fusion protein comprises a first CD40L subunit having the amino acid sequence of SEQ ID NO:21, a second CD40L subunit having the amino acid sequence of SEQ ID NO:21, and a third CD40L subunit having the amino acid sequence of SEQ ID NO:22. In one embodiment, a single chain trimeric CD40L fusion protein comprises a first CD40L subunit having the amino acid sequence of SEQ ID NO:21, a second CD40L subunit having the amino acid sequence of SEQ ID NO:22, and a third CD40L subunit having the amino acid sequence of SEQ ID NO:22. In one embodiment, a single chain trimeric CD40L fusion protein comprises a first CD40L subunit having the amino acid sequence of SEQ ID NO:21, a second CD40L subunit having the amino acid sequence of SEQ ID NO:33, and a third CD40L subunit having the amino acid sequence of SEQ ID NO:22. In one embodiment, a single chain trimeric CD40L fusion protein comprises a first CD40L subunit having the amino acid sequence of SEQ ID NO:21, a second CD40L subunit having the amino acid sequence of SEQ ID NO:34, and a third CD40L subunit having the amino acid sequence of SEQ ID NO:22. In one embodiment, a single chain trimeric CD40L fusion protein comprises a first CD40L subunit having the amino acid sequence of SEQ ID NO:22, a second CD40L subunit having the amino acid sequence of SEQ ID NO:20, and a third CD40L subunit having the amino acid sequence of SEQ ID NO:22. In one embodiment, a single chain trimeric CD40L fusion protein comprises a first CD40L subunit having the amino acid sequence of SEQ ID NO:22, a second CD40L subunit having the amino acid sequence of SEQ ID NO:21, and a third CD40L subunit having the amino acid sequence of SEQ ID NO:22. In one embodiment, a single chain trimeric CD40L fusion protein comprises a first CD40L subunit having the amino acid sequence of SEQ ID NO:22, a second CD40L subunit having the amino acid sequence of SEQ ID NO:22, and a third CD40L subunit having the amino acid sequence of SEQ ID NO:22. In one embodiment, a single chain trimeric CD40L fusion protein comprises a first CD40L subunit having the amino acid sequence of SEQ ID NO:22, a second CD40L subunit having the amino acid sequence of SEQ ID NO:33, and a third CD40L subunit having the amino acid sequence of SEQ ID NO:22. In one embodiment, a single chain trimeric CD40L fusion protein comprises a first CD40L subunit having the amino acid sequence of SEQ ID NO:22, a second CD40L subunit having the amino acid sequence of SEQ ID NO:34, and a third CD40L subunit having the amino acid sequence of SEQ ID NO:22. In one embodiment, a single chain trimeric CD40L fusion protein comprises a first CD40L subunit having the amino acid sequence of SEQ ID NO:33, a second CD40L subunit having the amino acid sequence of SEQ ID NO:20, and a third CD40L subunit having the amino acid sequence of SEQ ID NO:22. In one embodiment, a single chain trimeric CD40L fusion protein comprises a first CD40L subunit having the amino acid sequence of SEQ ID NO:33, a second CD40L subunit having the amino acid sequence of SEQ ID NO:21, and a third CD40L subunit having the amino acid sequence of SEQ ID NO:22. In one embodiment, a single chain trimeric CD40L fusion protein comprises a first CD40L subunit having the amino acid sequence of SEQ ID NO:33, a second CD40L subunit having the amino acid sequence of SEQ ID NO:22, and a third CD40L subunit having the amino acid sequence of SEQ ID NO:22. In one embodiment, a single chain trimeric CD40L fusion protein comprises a first CD40L subunit having the amino acid sequence of SEQ ID NO:33, a second CD40L subunit having the amino acid sequence of SEQ ID NO:33, and a third CD40L subunit having the amino acid sequence of SEQ ID NO:22. In one embodiment, a single chain trimeric CD40L fusion protein comprises a first CD40L subunit having the amino acid sequence of SEQ ID NO:33, a second CD40L subunit having the amino acid sequence of SEQ ID NO:34, and a third CD40L subunit having the amino acid sequence of SEQ ID NO:22. In one embodiment, a single chain trimeric CD40L fusion protein comprises a first CD40L subunit having the amino acid sequence of SEQ ID NO:34, a second CD40L subunit having the amino acid sequence of SEQ ID NO:20, and a third CD40L subunit having the amino acid sequence of SEQ ID NO:22. In one embodiment, a single chain trimeric CD40L fusion protein comprises a first CD40L subunit having the amino acid sequence of SEQ ID NO:34, a second CD40L subunit having the amino acid sequence of SEQ ID NO:21, and a third CD40L subunit having the amino acid sequence of SEQ ID NO:22. In one embodiment, a single chain trimeric CD40L fusion protein comprises a first CD40L subunit having the amino acid sequence of SEQ ID NO:34, a second CD40L subunit having the amino acid sequence of SEQ ID NO:22, and a third CD40L subunit having the amino acid sequence of SEQ ID NO:22. In one embodiment, a single chain trimeric CD40L fusion protein comprises a first CD40L subunit having the amino acid sequence of SEQ ID NO:34, a second CD40L subunit having the amino acid sequence of SEQ ID NO:33, and a third CD40L subunit having the amino acid sequence of SEQ ID NO:22. In one embodiment, a single chain trimeric CD40L fusion protein comprises a first CD40L subunit having the amino acid sequence of SEQ ID NO:34, a second CD40L subunit having the amino acid sequence of SEQ ID NO:34, and a third CD40L subunit having the amino acid sequence of SEQ ID NO:22.

In one embodiment, a single chain trimeric CD40L fusion protein comprises a first CD40L subunit having the amino acid sequence of SEQ ID NO:20, a second CD40L subunit having the amino acid sequence of SEQ ID NO:20, and a third CD40L subunit having the amino acid sequence of SEQ ID NO:33. In one embodiment, a single chain trimeric CD40L fusion protein comprises a first CD40L subunit having the amino acid sequence of SEQ ID NO:20, a second CD40L subunit having the amino acid sequence of SEQ ID NO:21, and a third CD40L subunit having the amino acid sequence of SEQ ID NO:33. In one embodiment, a single chain trimeric CD40L fusion protein comprises a first CD40L subunit having the amino acid sequence of SEQ ID NO:20, a second CD40L subunit having the amino acid sequence of SEQ ID NO:22, and a third CD40L subunit having the amino acid sequence of SEQ ID NO:33. In one embodiment, a single chain trimeric CD40L fusion protein comprises a first CD40L subunit having the amino acid sequence of SEQ ID NO:20, a second CD40L subunit having the amino acid sequence of SEQ ID NO:33, and a third CD40L subunit having the amino acid sequence of SEQ ID NO:33. In one embodiment, a single chain trimeric CD40L fusion protein comprises a first CD40L subunit having the amino acid sequence of SEQ ID NO:20, a second CD40L subunit having the amino acid sequence of SEQ ID NO:34, and a third CD40L subunit having the amino acid sequence of SEQ ID NO:33. In one embodiment, a single chain trimeric CD40L fusion protein comprises a first CD40L subunit having the amino acid sequence of SEQ ID NO:21, a second CD40L subunit having the amino acid sequence of SEQ ID NO:20, and a third CD40L subunit having the amino acid sequence of SEQ ID NO:33. In one embodiment, a single chain trimeric CD40L fusion protein comprises a first CD40L subunit having the amino acid sequence of SEQ ID NO:21, a second CD40L subunit having the amino acid sequence of SEQ ID NO:21, and a third CD40L subunit having the amino acid sequence of SEQ ID NO:33. In one embodiment, a single chain trimeric CD40L fusion protein comprises a first CD40L subunit having the amino acid sequence of SEQ ID NO:21, a second CD40L subunit having the amino acid sequence of SEQ ID NO:22, and a third CD40L subunit having the amino acid sequence of SEQ ID NO:33. In one embodiment, a single chain trimeric CD40L fusion protein comprises a first CD40L subunit having the amino acid sequence of SEQ ID NO:21, a second CD40L subunit having the amino acid sequence of SEQ ID NO:33, and a third CD40L subunit having the amino acid sequence of SEQ ID NO:33. In one embodiment, a single chain trimeric CD40L fusion protein comprises a first CD40L subunit having the amino acid sequence of SEQ ID NO:21, a second CD40L subunit having the amino acid sequence of SEQ ID NO:34, and a third CD40L subunit having the amino acid sequence of SEQ ID NO:33. In one embodiment, a single chain trimeric CD40L fusion protein comprises a first CD40L subunit having the amino acid sequence of SEQ ID NO:22, a second CD40L subunit having the amino acid sequence of SEQ ID NO:20, and a third CD40L subunit having the amino acid sequence of SEQ ID NO:33. In one embodiment, a single chain trimeric CD40L fusion protein comprises a first CD40L subunit having the amino acid sequence of SEQ ID NO:22, a second CD40L subunit having the amino acid sequence of SEQ ID NO:21, and a third CD40L subunit having the amino acid sequence of SEQ ID NO:33. In one embodiment, a single chain trimeric CD40L fusion protein comprises a first CD40L subunit having the amino acid sequence of SEQ ID NO:22, a second CD40L subunit having the amino acid sequence of SEQ ID NO:22, and a third CD40L subunit having the amino acid sequence of SEQ ID NO:33. In one embodiment, a single chain trimeric CD40L fusion protein comprises a first CD40L subunit having the amino acid sequence of SEQ ID NO:22, a second CD40L subunit having the amino acid sequence of SEQ ID NO:33, and a third CD40L subunit having the amino acid sequence of SEQ ID NO:33. In one embodiment, a single chain trimeric CD40L fusion protein comprises a first CD40L subunit having the amino acid sequence of SEQ ID NO:22, a second CD40L subunit having the amino acid sequence of SEQ ID NO:34, and a third CD40L subunit having the amino acid sequence of SEQ ID NO:33. In one embodiment, a single chain trimeric CD40L fusion protein comprises a first CD40L subunit having the amino acid sequence of SEQ ID NO:33, a second CD40L subunit having the amino acid sequence of SEQ ID NO:20, and a third CD40L subunit having the amino acid sequence of SEQ ID NO:33. In one embodiment, a single chain trimeric CD40L fusion protein comprises a first CD40L subunit having the amino acid sequence of SEQ ID NO:33, a second CD40L subunit having the amino acid sequence of SEQ ID NO:21, and a third CD40L subunit having the amino acid sequence of SEQ ID NO:33. In one embodiment, a single chain trimeric CD40L fusion protein comprises a first CD40L subunit having the amino acid sequence of SEQ ID NO:33, a second CD40L subunit having the amino acid sequence of SEQ ID NO:22, and a third CD40L subunit having the amino acid sequence of SEQ ID NO:33. In one embodiment, a single chain trimeric CD40L fusion protein comprises a first CD40L subunit having the amino acid sequence of SEQ ID NO:33, a second CD40L subunit having the amino acid sequence of SEQ ID NO:33, and a third CD40L subunit having the amino acid sequence of SEQ ID NO:33. In one embodiment, a single chain trimeric CD40L fusion protein comprises a first CD40L subunit having the amino acid sequence of SEQ ID NO:33, a second CD40L subunit having the amino acid sequence of SEQ ID NO:34, and a third CD40L subunit having the amino acid sequence of SEQ ID NO:33. In one embodiment, a single chain trimeric CD40L fusion protein comprises a first CD40L subunit having the amino acid sequence of SEQ ID NO:34, a second CD40L subunit having the amino acid sequence of SEQ ID NO:20, and a third CD40L subunit having the amino acid sequence of SEQ ID NO:33. In one embodiment, a single chain trimeric CD40L fusion protein comprises a first CD40L subunit having the amino acid sequence of SEQ ID NO:34, a second CD40L subunit having the amino acid sequence of SEQ ID NO:21, and a third CD40L subunit having the amino acid sequence of SEQ ID NO:33. In one embodiment, a single chain trimeric CD40L fusion protein comprises a first CD40L subunit having the amino acid sequence of SEQ ID NO:34, a second CD40L subunit having the amino acid sequence of SEQ ID NO:22, and a third CD40L subunit having the amino acid sequence of SEQ ID NO:33. In one embodiment, a single chain trimeric CD40L fusion protein comprises a first CD40L subunit having the amino acid sequence of SEQ ID NO:34, a second CD40L subunit having the amino acid sequence of SEQ ID NO:33, and a third CD40L subunit having the amino acid sequence of SEQ ID NO:33. In one embodiment, a single chain trimeric CD40L fusion protein comprises a first CD40L subunit having the amino acid sequence of SEQ ID NO:34, a second CD40L subunit having the amino acid sequence of SEQ ID NO:34, and a third CD40L subunit having the amino acid sequence of SEQ ID NO:33.

In one embodiment, a single chain trimeric CD40L fusion protein comprises a first CD40L subunit having the amino acid sequence of SEQ ID NO:20, a second CD40L subunit having the amino acid sequence of SEQ ID NO:20, and a third CD40L subunit having the amino acid sequence of SEQ ID NO:34. In one embodiment, a single chain trimeric CD40L fusion protein comprises a first CD40L subunit having the amino acid sequence of SEQ ID NO:20, a second CD40L subunit having the amino acid sequence of SEQ ID NO:21, and a third CD40L subunit having the amino acid sequence of SEQ ID NO:34. In one embodiment, a single chain trimeric CD40L fusion protein comprises a first CD40L subunit having the amino acid sequence of SEQ ID NO:20, a second CD40L subunit having the amino acid sequence of SEQ ID NO:22, and a third CD40L subunit having the amino acid sequence of SEQ ID NO:34. In one embodiment, a single chain trimeric CD40L fusion protein comprises a first CD40L subunit having the amino acid sequence of SEQ ID NO:20, a second CD40L subunit having the amino acid sequence of SEQ ID NO:33, and a third CD40L subunit having the amino acid sequence of SEQ ID NO:34. In one embodiment, a single chain trimeric CD40L fusion protein comprises a first CD40L subunit having the amino acid sequence of SEQ ID NO:20, a second CD40L subunit having the amino acid sequence of SEQ ID NO:34, and a third CD40L subunit having the amino acid sequence of SEQ ID NO:34. In one embodiment, a single chain trimeric CD40L fusion protein comprises a first CD40L subunit having the amino acid sequence of SEQ ID NO:21, a second CD40L subunit having the amino acid sequence of SEQ ID NO:20, and a third CD40L subunit having the amino acid sequence of SEQ ID NO:34. In one embodiment, a single chain trimeric CD40L fusion protein comprises a first CD40L subunit having the amino acid sequence of SEQ ID NO:21, a second CD40L subunit having the amino acid sequence of SEQ ID NO:21, and a third CD40L subunit having the amino acid sequence of SEQ ID NO:34. In one embodiment, a single chain trimeric CD40L fusion protein comprises a first CD40L subunit having the amino acid sequence of SEQ ID NO:21, a second CD40L subunit having the amino acid sequence of SEQ ID NO:22, and a third CD40L subunit having the amino acid sequence of SEQ ID NO:34. In one embodiment, a single chain trimeric CD40L fusion protein comprises a first CD40L subunit having the amino acid sequence of SEQ ID NO:21, a second CD40L subunit having the amino acid sequence of SEQ ID NO:33, and a third CD40L subunit having the amino acid sequence of SEQ ID NO:34. In one embodiment, a single chain trimeric CD40L fusion protein comprises a first CD40L subunit having the amino acid sequence of SEQ ID NO:21, a second CD40L subunit having the amino acid sequence of SEQ ID NO:34, and a third CD40L subunit having the amino acid sequence of SEQ ID NO:34. In one embodiment, a single chain trimeric CD40L fusion protein comprises a first CD40L subunit having the amino acid sequence of SEQ ID NO:22, a second CD40L subunit having the amino acid sequence of SEQ ID NO:20, and a third CD40L subunit having the amino acid sequence of SEQ ID NO:34. In one embodiment, a single chain trimeric CD40L fusion protein comprises a first CD40L subunit having the amino acid sequence of SEQ ID NO:22, a second CD40L subunit having the amino acid sequence of SEQ ID NO:21, and a third CD40L subunit having the amino acid sequence of SEQ ID NO:34. In one embodiment, a single chain trimeric CD40L fusion protein comprises a first CD40L subunit having the amino acid sequence of SEQ ID NO:22, a second CD40L subunit having the amino acid sequence of SEQ ID NO:22, and a third CD40L subunit having the amino acid sequence of SEQ ID NO:34. In one embodiment, a single chain trimeric CD40L fusion protein comprises a first CD40L subunit having the amino acid sequence of SEQ ID NO:22, a second CD40L subunit having the amino acid sequence of SEQ ID NO:33, and a third CD40L subunit having the amino acid sequence of SEQ ID NO:34. In one embodiment, a single chain trimeric CD40L fusion protein comprises a first CD40L subunit having the amino acid sequence of SEQ ID NO:22, a second CD40L subunit having the amino acid sequence of SEQ ID NO:34, and a third CD40L subunit having the amino acid sequence of SEQ ID NO:34. In one embodiment, a single chain trimeric CD40L fusion protein comprises a first CD40L subunit having the amino acid sequence of SEQ ID NO:33, a second CD40L subunit having the amino acid sequence of SEQ ID NO:20, and a third CD40L subunit having the amino acid sequence of SEQ ID NO:34. In one embodiment, a single chain trimeric CD40L fusion protein comprises a first CD40L subunit having the amino acid sequence of SEQ ID NO:33, a second CD40L subunit having the amino acid sequence of SEQ ID NO:21, and a third CD40L subunit having the amino acid sequence of SEQ ID NO:34. In one embodiment, a single chain trimeric CD40L fusion protein comprises a first CD40L subunit having the amino acid sequence of SEQ ID NO:33, a second CD40L subunit having the amino acid sequence of SEQ ID NO:22, and a third CD40L subunit having the amino acid sequence of SEQ ID NO:34. In one embodiment, a single chain trimeric CD40L fusion protein comprises a first CD40L subunit having the amino acid sequence of SEQ ID NO:33, a second CD40L subunit having the amino acid sequence of SEQ ID NO:33, and a third CD40L subunit having the amino acid sequence of SEQ ID NO:34. In one embodiment, a single chain trimeric CD40L fusion protein comprises a first CD40L subunit having the amino acid sequence of SEQ ID NO:33, a second CD40L subunit having the amino acid sequence of SEQ ID NO:34, and a third CD40L subunit having the amino acid sequence of SEQ ID NO:34. In one embodiment, a single chain trimeric CD40L fusion protein comprises a first CD40L subunit having the amino acid sequence of SEQ ID NO:34, a second CD40L subunit having the amino acid sequence of SEQ ID NO:20, and a third CD40L subunit having the amino acid sequence of SEQ ID NO:34. In one embodiment, a single chain trimeric CD40L fusion protein comprises a first CD40L subunit having the amino acid sequence of SEQ ID NO:34, a second CD40L subunit having the amino acid sequence of SEQ ID NO:21, and a third CD40L subunit having the amino acid sequence of SEQ ID NO:34. In one embodiment, a single chain trimeric CD40L fusion protein comprises a first CD40L subunit having the amino acid sequence of SEQ ID NO:34, a second CD40L subunit having the amino acid sequence of SEQ ID NO:22, and a third CD40L subunit having the amino acid sequence of SEQ ID NO:34. In one embodiment, a single chain trimeric CD40L fusion protein comprises a first CD40L subunit having the amino acid sequence of SEQ ID NO:34, a second CD40L subunit having the amino acid sequence of SEQ ID NO:33, and a third CD40L subunit having the amino acid sequence of SEQ ID NO:34. In one embodiment, a single chain trimeric CD40L fusion protein comprises a first CD40L subunit having the amino acid sequence of SEQ ID NO:34, a second CD40L subunit having the amino acid sequence of SEQ ID NO:34, and a third CD40L subunit having the amino acid sequence of SEQ ID NO:34.

In some embodiments, the single chain trimeric CD40L fusion protein comprises three CD40L subunits, connected to each other with flexible peptide linkers to form a linear CD40L trimer. In particular embodiments, the flexible peptide linkers connecting the CD40L subunits in the fusion protein comprise any one sequence selected from the group consisting of EGKSSGSGS (SEQ ID NO:23) and (G₃S)ₙ (SEQ ID NO:39), wherein n is an integer from 1 to 20. In some embodiments, the integer is 1. In some embodiments, the integer is 2. In some embodiments, the integer is 3. In some embodiments, the integer is 4. In some embodiments, the integer is 5. In some embodiments, the integer is 6. In some embodiments, the integer is 7. In some embodiments, the integer is 8. In some embodiments, the integer is 9. In some embodiments, the integer is 10. In some embodiments, the integer is 11. In some embodiments, the integer is 12. In some embodiments, the integer is 13. In some embodiments, the integer is 14. In some embodiments, the integer is 15. In some embodiments, the integer is 16. In some embodiments, the integer is 17. In some embodiments, the integer is 18. In some embodiments, the integer is 19. In some embodiments, the integer is 20.

In some embodiments, the flexible linker comprises EGKSSGSGS (SEQ ID NO:23). In some embodiments, the flexible linker comprises (G₃S)₃ (SEQ ID NO:25). In some embodiments, the peptide linker comprises SEQ ID NO:23. In some embodiments, the peptide linker comprises SEQ ID NO:25.

In some embodiments, the peptide linker comprises the sequence EGKSSGSGS (SEQ ID NO:23). In some embodiments, the peptide linker comprises an amino acid sequence having at least 50%, at least 55%, at least 60%, at least 65%, at least 70%, at least 75%, at least 80%, at least 85%, at least 90%, at least 95%, at least 98% or at least 99% sequence identity with EGKSSGSGS (SEQ ID NO:23). In some embodiments, the peptide linker comprises an amino acid sequence having at least 50% identity. In some embodiments, the peptide linker comprises an amino acid sequence having at least 55% identity. In some embodiments, the peptide linker comprises an amino acid sequence having at least 60% identity. In some embodiments, the peptide linker comprises an amino acid sequence having at least 65% identity. In some embodiments, the peptide linker comprises an amino acid sequence having at least 70% identity. In some embodiments, the peptide linker comprises an amino acid sequence having at least 75% identity. In some embodiments, the peptide linker comprises an amino acid sequence having at least 80% identity. In some embodiments, the peptide linker comprises an amino acid sequence having at least 85% identity. In some embodiments, the peptide linker comprises an amino acid sequence having at least 90% identity. In some embodiments, the peptide linker comprises an amino acid sequence having at least 95% identity. In some embodiments, the peptide linker comprises an amino acid sequence having at least 98% identity. In some embodiments, the peptide linker comprises an amino acid sequence having at least 99% identity. In some embodiments, the peptide linker comprises an amino acid sequence having 100% sequence identity.

In some embodiments, the peptide linker comprises the sequence (G₃S)ₙ (SEQ ID NO:39), or comprises an amino acid sequence having at least 50%, at least 55%, at least 60%, at least 65%, at least 70%, at least 75%, at least 80%, at least 85%, at least 90%, at least 95%, at least 98% or at least 99% sequence identity with (G₃S)ₙ (SEQ ID NO:39). In some embodiments, the peptide linker comprises an amino acid sequence having at least 50% identity. In some embodiments, the peptide linker comprises an amino acid sequence having at least 55% identity. In some embodiments, the peptide linker comprises an amino acid sequence having at least 60% identity. In some embodiments, the peptide linker comprises an amino acid sequence having at least 65% identity. In some embodiments, the peptide linker comprises an amino acid sequence having at least 70% identity. In some embodiments, the peptide linker comprises an amino acid sequence having at least 75% identity. In some embodiments, the peptide linker comprises an amino acid sequence having at least 80% identity. In some embodiments, the peptide linker comprises an amino acid sequence having at least 85% identity. In some embodiments, the peptide linker comprises an amino acid sequence having at least 90% identity. In some embodiments, the peptide linker comprises an amino acid sequence having at least 95% identity. In some embodiments, the peptide linker comprises an amino acid sequence having at least 98% identity. In some embodiments, the peptide linker comprises an amino acid sequence having at least 99% identity. In some embodiments, the peptide linker comprises an amino acid sequence having 100% sequence identity.

In particular embodiments, the single chain trimeric CD40L fusion protein comprises any one sequence selected from the sequences shown in the following table, where the underlined portion corresponds to the linker sequences:

| | |
|---|---|
| CD40L Trimer v.1 | NPQIAAHVISEASSKTTSVLQWAEKGYYTMSNNLVTLENGKQLTV |
| (SEQ ID NO: 35) | KRQGLYYIYAQVTFCSNREASSQAPFIASLWLKSPGRFERILLRAA |
| | NTHSSAKPCGQQSIHLGGVFELQPGASVFVNVTDPSQVSHGTGFTS |
| | FGLLKLEGKSSGSGSQIAAHVISEASSKTTSVLQWAEKGYYTMSN |
| | NLVTLENGKQLTVKRQGLYYIYAQVTFCSNREASSQAPFIASLWL |
| | KSPGRFERILLRAANTHSSAKPCGQQSIHLGGVFELQPGASVFVNV |
| | TDPSQVSHGTGFTSFGLLKLEGKSSGSGSQIAAHVISEASSKTTSVL |
| | QWAEKGYYTMSNNLVTLENGKQLTVKRQGLYYIYAQVTFCSNR |
| | EASSQAPFIASLWLKSPGRFERILLRAANTHSSAKPCGQQSIHLGG |
| | VFELQPGASVFVNVTDPSQVSHGTGFTSFGLLKL |
| CD40L Trimer v.2 | NPQIAAHVISEASSKTTSVLQWAEKGYYTMSNNLVTLENGKQLTV |
| (SEQ ID NO: 36) | KRQGLYYIYAQVTFCSNREASSQAPFIASLCLKSPGRFERILLRAAN |
| | THSSAKPCGQQSIHLGGVFELQPGASVFVNVTDPSQVSHGTGFTSF |
| | GLLKLGGGSGGGSGGGSNPQIAAHVISEASSKTTSVLQWAEKGYY |
| | TMSNNLVTLENGKQLTVKRQGLYYIYAQVTFCSNREASSQAPFIA |
| | SLCLKSPGRFERILLRAANTHSSAKPCGQQSIHLGGVFELQPGASV |
| | FVNVTDPSQVSHGTGFTSFGLLKLGGGSGGGSGGGSNPQIAAHVIS |
| | EASSKTTSVLQWAEKGYYTMSNNLVTLENGKQLTVKRQGLYYIY |
| | AQVTFCSNREASSQAPFIASLCLKSPGRFERILLRAANTHSSAKPCG |
| | QQSIHLGGVFELQPGASVFVNVTDPSQVSHGTGFTSFGLLKL |
| CD40L Trimer v.3 | NPQIAAHVISEASSKTTSVLQWAEKGYYTMSNNLVTLENGKQLTV |
| (SEQ ID NO: 37) | KRQGLYYIYAQVTFCSNREASSQAPFIASLCLKSPGRFERILLRAAN |
| | THSSAKPCGQQSIHLGGVFELQPGASVFVNVTDPSQVSHGTGFTSF |
| | GLLKLEGKSSGSGSNPQIAAHVISEASSKTTSVLQWAEKGYYTMS |
| | NNLVTLENGKQLTVKRQGLYYIYAQVTFCSNREASSQAPFIASLCL |
| | KSPGRFERILLRAANTHSSAKPCGQQSIHLGGVFELQPGASVFVNV |
| | TDPSQVSHGTGFTSFGLLKLEGKSSGSGSNPQIAAHVISEASSKTTS |
| | VLQWAEKGYYTMSNNLVTLENGKQLTVKRQGLYYIYAQVTFCS |
| | NREASSQAPFIASLCLKSPGRFERILLRAANTHSSAKPCGQQSIHLG |
| | GVFELQPGASVFVNVTDPSQVSHGTGFTSFGLLKL |
| CD40L Trimer v.4 | IAAHVISEASSKTTSVLQWAEKGYYTMSNNLVTLENGKQLTVKR |
| (SEQ ID NO: 38) | QGLYYIYAQVTFCSNREASSQAPFIASLCLKSPGRFERILLRAANTH |
| | SSAKPCGQQSIHLGGVFELQPGASVFVNVTDPSQVSHGTGFTSFGL |
| | LKLEGKSSGSGSQIAAHVISEASSKTTSVLQWAEKGYYTMSNNLV |
| | TLENGKQLTVKRQGLYYIYAQVTFCSNREASSQAPFIASLCLKSPG |
| | RFERILLRAANTHSSAKPCGQQSIHLGGVFELQPGASVFVNVTDPS |
| | QVSHGTGFTSFGLLKLEGKSSGSGSQIAAHVISEASSKTTSVLQWA |
| | EKGYYTMSNNLVTLENGKQLTVKRQGLYYIYAQVTFCSNREASS |
| | QAPFIASLCLKSPGRFERILLRAANTHSSAKPCGQQSIHLGGVFELQ |
| | PGASVFVNVTDPSQVSHGTGFTSFGLLKL |

In one embodiment, the single chain trimeric CD40L fusion protein comprises SEQ ID NO:35. In another embodiment, the single chain trimeric CD40L fusion protein comprises SEQ ID NO:36. In another embodiment, the single chain trimeric CD40L fusion protein comprises SEQ ID NO:37. In another embodiment, the single chain trimeric CD40L fusion protein comprises SEQ ID NO:38.

In some embodiments, the single chain trimeric CD40L fusion protein can also be recombinantly fused, e.g., to a heterologous peptide or polypeptide (or fragment thereof, for example, to a polypeptide of about 10, about 20, about 30, about 40, about 50, about 60, about 70, about 80, about 90, or about 100 amino acids) to generate larger fusion proteins. Accordingly, in some embodiments, provided herein are polypeptides comprising a CD40L trimer fused to a heterologous peptide or polypeptide not derived from CD40L (which is referred to as a “non-CD40L” peptide or polypeptide sometimes in this application). The connection between the CD40L trimer and the non-CD40L peptide or polypeptide can be direct or via a flexible peptide tether. In some embodiments, the non-CD40L peptide or polypeptide is fused to the N-terminus of the CD40L trimer. In some embodiments, the non-CD40L peptide or polypeptide is fused to the C-terminus of the CD40L trimer.

Accordingly, in one aspect, provided herein are fusion proteins comprising a CD40L trimer fused to a non-CD40L peptide or polypeptide, either directly or via a flexible peptide tether. In some embodiments, the non-CD40L portion of the fusion protein comprises an Fc monomer peptide.

Provided herein is a single chain trimeric CD40L Fc fusion protein comprising (a) three CD40L subunits covalently linked to one another by peptide linkers; and (b) an Fc monomer peptide.

As provided herein, the single chain trimeric CD40L Fc fusion protein can comprise an Fc region from an IgG antibody. In some embodiments, the single chain trimeric CD40L Fc fusion protein can comprise an Fc region from an IgG1, IgG2, IgG3, or IgG4 antibody. In some embodiments, the single chain trimeric CD40L Fc fusion protein can comprise an Fc region from an IgG1 antibody. In some embodiments, the single chain trimeric CD40L Fc fusion protein can comprise an Fc region from an IgG2 antibody. In some embodiments, the single chain trimeric CD40L Fc fusion protein can comprise an Fc region from an IgG3 antibody. In some embodiments, the single chain trimeric CD40L Fc fusion protein can comprise an Fc region from an IgG4 antibody. In some embodiments, the single chain trimeric CD40L Fc fusion protein can comprise an Fc monomer that is a silent Fc region or a modified Fc region, that has a genetically engineered Fc domain with key mutations that abrogate binding of Fc receptors and abolish antibody directed cellular cytotoxicity (ADCC) effector function.

In some embodiments, the trimeric CD40L portion of the fusion protein is genetically fused or chemically conjugated to the C-terminus of the Fc region. In some embodiments, the trimeric CD40L portion of the fusion protein is genetically fused or chemically conjugated to the N-terminus of the Fc region.

In some embodiments, the Fc monomer peptide is covalently linked to the CD40L trimer by a peptide tether.

As used herein, the term “tether” or “peptide tether” refers to the amino acid sequence that connects the trimeric CD40L portion of the fusion protein to the Fc monomer peptide. In some embodiments, the peptide tether comprises between 0 and 20 amino acids (inclusive of the end points). In some embodiments, the peptide tether comprises between 0 and 1 amino acids. In some embodiments, the peptide tether comprises between 0 and 5 amino acids. In some embodiments, the peptide tether comprises between 0 and 10 amino acids. In some embodiments, the peptide tether comprises between 0 and 15 amino acids. In some embodiments, the peptide tether comprises between 5 and 10 amino acids. In some embodiments, the peptide tether comprises between 5 and 15 amino acids. In some embodiments, the peptide tether comprises between 5 and 20 amino acids. In some embodiments, the peptide tether comprises between 10 and 15 amino acids. In some embodiments, the peptide tether comprises between 10 and 20 amino acids. In contrast, the term “linker” or peptide linker” refers to the amino acid sequence that connects the CD40L subunits to each other to form a trimer.

In one embodiment, the peptide tether that connects the trimeric CD40L portion of the fusion protein to the Fc monomer peptide has the sequence (G₄S)ₙ (SEQ ID NO:40), wherein n is an integer from 0 to 20. In some embodiments, the integer is 1. In some embodiments, the integer is 2. In some embodiments, the integer is 3. In some embodiments, the integer is 4. In some embodiments, the integer is 5. In some embodiments, the integer is 6. In some embodiments, the integer is 7. In some embodiments, the integer is 8. In some embodiments, the integer is 9. In some embodiments, the integer is 10. In some embodiments, the integer is 11. In some embodiments, the integer is 12. In some embodiments, the integer is 13. In some embodiments, the integer is 14. In some embodiments, the integer is 15. In some embodiments, the integer is 16. In some embodiments, the integer is 17. In some embodiments, the integer is 18. In some embodiments, the integer is 19. In some embodiments, the integer is 20.

In one embodiment, the flexible tether does not comprise a (G₄S) (SEQ ID NO:28) motif. In one embodiment, the flexible tether comprises (G₄S)₁ (SEQ ID NO:28). In one embodiment, the flexible tether comprises (G₄S)₂ (SEQ ID NO:26). In one embodiment, the flexible tether comprises (G₄S)₃ (SEQ ID NO:24). In one embodiment, the flexible tether comprises (G₄S)₄ (SEQ ID NO:27). In some embodiments, the peptide tether comprises SEQ ID NO:24. In some embodiments, the peptide tether comprises SEQ ID NO:26. In some embodiments, the peptide tether comprises SEQ ID NO:27. In some embodiments, the peptide tether comprises SEQ ID NO:28.

In some embodiments, the peptide tether comprises the sequence (G₄S)ₙ (SEQ ID NO:40), or comprises an amino acid sequence having at least 50, at least 55, at least 60, at least 65, at least 70, at least 75, at least 80, at least 85, at least 90, at least 95, at least 98 or at least 99%, sequence identity with (G₄S)ₙ (SEQ ID NO:40). In some embodiments, the peptide linker comprises an amino acid sequence having at least 50% identity with (G₄S)ₙ (SEQ ID NO:40). In some embodiments, the peptide linker comprises an amino acid sequence having at least 55% identity with (G₄S)ₙ (SEQ ID NO:40). In some embodiments, the peptide linker comprises an amino acid sequence having at least 60% identity with (G₄S)ₙ (SEQ ID NO:40). In some embodiments, the peptide linker comprises an amino acid sequence having at least 65% identity with (G₄S)ₙ (SEQ ID NO:40). In some embodiments, the peptide linker comprises an amino acid sequence having at least 70% identity with (G₄S)ₙ (SEQ ID NO:40). In some embodiments, the peptide linker comprises an amino acid sequence having at least 75% identity with (G₄S)ₙ (SEQ ID NO:40). In some embodiments, the peptide linker comprises an amino acid sequence having at least 80% identity with (G₄S)ₙ (SEQ ID NO:40). In some embodiments, the peptide linker comprises an amino acid sequence having at least 85% identity with (G₄S)ₙ (SEQ ID NO:40). In some embodiments, the peptide linker comprises an amino acid sequence having at least 90% identity with (G₄S)ₙ (SEQ ID NO:40). In some embodiments, the peptide linker comprises an amino acid sequence having at least 95% identity with (G₄S)ₙ (SEQ ID NO:40). In some embodiments, the peptide linker comprises an amino acid sequence having at least 98% identity with (G₄S)ₙ (SEQ ID NO:40). In some embodiments, the peptide linker comprises an amino acid sequence having at least 99% identity with (G₄S)ₙ (SEQ ID NO:40). In some embodiments, the peptide linker comprises an amino acid sequence having 100% sequence identity with (G₄S)ₙ (SEQ ID NO:40).

In one embodiment, the peptide linker that connects the CD40L subunits to each other to form the trimeric CD40L portion of the fusion protein may be a flexible linker comprising a sequence selected from the group consisting of EGKSSGSGS (SEQ ID NO:23) and (G₃S)ₙ (SEQ ID NO:39), wherein n is an integer from 1 to 20. In some embodiments, the integer is 1. In some embodiments, the integer is 2. In some embodiments, the integer is 3. In some embodiments, the integer is 4. In some embodiments, the integer is 5. In some embodiments, the integer is 6. In some embodiments, the integer is 7. In some embodiments, the integer is 8. In some embodiments, the integer is 9. In some embodiments, the integer is 10. In some embodiments, the integer is 11. In some embodiments, the integer is 12. In some embodiments, the integer is 13. In some embodiments, the integer is 14. In some embodiments, the integer is 15. In some embodiments, the integer is 16. In some embodiments, the integer is 17. In some embodiments, the integer is 18. In some embodiments, the integer is 19. In some embodiments, the integer is 20. In one embodiment, the flexible linker comprises EGKSSGSGS (SEQ ID NO:23). In one embodiment, the flexible linker comprises (G₃S)₃ (SEQ ID NO:25). In some embodiments, the peptide linker comprises SEQ ID NO:23. In some embodiments, the peptide linker comprises SEQ ID NO:25.

In some embodiments, the peptide linker comprises the sequence EGKSSGSGS(SEQ ID NO:23), or comprises an amino acid sequence having at least 50, at least 55, at least 60, at least 65, at least 70, at least 75, at least 80, at least 85, at least 90, at least 95, at least 98 or at least 99%, sequence identity with EGKSSGSGS (SEQ ID NO:23). In some embodiments, the peptide linker comprises an amino acid sequence having at least 50% identity with EGKSSGSGS (SEQ ID NO:23). In some embodiments, the peptide linker comprises an amino acid sequence having at least 55% identity with EGKSSGSGS (SEQ ID NO:23). In some embodiments, the peptide linker comprises an amino acid sequence having at least 60% identity with EGKSSGSGS (SEQ ID NO:23). In some embodiments, the peptide linker comprises an amino acid sequence having at least 65% identity with EGKSSGSGS (SEQ ID NO:23). In some embodiments, the peptide linker comprises an amino acid sequence having at least 70% identity with EGKSSGSGS (SEQ ID NO:23). In some embodiments, the peptide linker comprises an amino acid sequence having at least 75% identity with EGKSSGSGS (SEQ ID NO:23). In some embodiments, the peptide linker comprises an amino acid sequence having at least 80% identity with EGKSSGSGS (SEQ ID NO:23). In some embodiments, the peptide linker comprises an amino acid sequence having at least 85% identity with EGKSSGSGS (SEQ ID NO:23). In some embodiments, the peptide linker comprises an amino acid sequence having at least 90% identity with EGKSSGSGS (SEQ ID NO:23). In some embodiments, the peptide linker comprises an amino acid sequence having at least 95% identity with EGKSSGSGS (SEQ ID NO:23). In some embodiments, the peptide linker comprises an amino acid sequence having at least 98% identity with EGKSSGSGS (SEQ ID NO:23). In some embodiments, the peptide linker comprises an amino acid sequence having at least 99% identity with EGKSSGSGS (SEQ ID NO:23). In some embodiments, the peptide linker comprises an amino acid sequence having 100% sequence identity with EGKSSGSGS (SEQ ID NO:23).

In some embodiments, the peptide linker comprises the sequence (G₃S)ₙ (SEQ ID NO:39), or comprises an amino acid sequence having at least 50, at least 55, at least 60, at least 65, at least 70, at least 75, at least 80, at least 85, at least 90, at least 95, at least 98 or at least 99%, sequence identity with (G₃S)ₙ (SEQ ID NO:39). In some embodiments, the peptide linker comprises an amino acid sequence having at least 50% identity with (G₃S)ₙ (SEQ ID NO:39). In some embodiments, the peptide linker comprises an amino acid sequence having at least 55% identity with (G₃S)ₙ (SEQ ID NO:39). In some embodiments, the peptide linker comprises an amino acid sequence having at least 60% identity with (G₃S)ₙ (SEQ ID NO:39). In some embodiments, the peptide linker comprises an amino acid sequence having at least 65% identity with (G₃S)ₙ (SEQ ID NO:39). In some embodiments, the peptide linker comprises an amino acid sequence having at least 70% identity with (G₃S)ₙ (SEQ ID NO:39). In some embodiments, the peptide linker comprises an amino acid sequence having at least 75% identity with (G₃S)ₙ (SEQ ID NO:39). In some embodiments, the peptide linker comprises an amino acid sequence having at least 80% identity with (G₃S)ₙ (SEQ ID NO:39). In some embodiments, the peptide linker comprises an amino acid sequence having at least 85% identity with (G₃S)ₙ (SEQ ID NO:39). In some embodiments, the peptide linker comprises an amino acid sequence having at least 90% identity with (G₃S)ₙ (SEQ ID NO:39). In some embodiments, the peptide linker comprises an amino acid sequence having at least 95% identity with (G₃S)ₙ (SEQ ID NO:39). In some embodiments, the peptide linker comprises an amino acid sequence having at least 98% identity with (G₃S)ₙ (SEQ ID NO:39). In some embodiments, the peptide linker comprises an amino acid sequence having at least 99% identity with (G₃S)ₙ (SEQ ID NO:39). In some embodiments, the peptide linker comprises an amino acid sequence having 100% sequence identity with (G₃S)ₙ (SEQ ID NO:39).

In particular embodiments, the Fc monomer in the fusion protein according to the present disclosure comprises any one sequence selected from:

| | |
|---|---|
| Fc Monomer v.1 | VERKSCVECPPCPAPPVAGPSVFLFPPKPKDTLMISRTPEVTCVVV |
| (SEQ ID NO: 29) | DVSHEDPEVQFNWYVDGVEVHNAKTKPREEQFNSTFRVVSVLTV |
| | VHQDWLNGKEYKCKVSNKGLPAPIEKTISKTKGQPREPQVYTLPP |
| | SREEMTKNQVSLTCLVKGFYPSDIAVEWESNGQPENNYKTTPPML |
| | DSDGSFFLYSKLTVDKSRWQQGNVFSCSVMHEALHNHYTQKSLS |
| | LSPGK |
| Fc Monomer v.2 | EPKSSDKTHTCPPCPAPEAAGGPSVFLFPPKPKDTLMISRTPEVTCV |
| (SEQ ID NO: 30) | VVSVSHEDPEVKFNWYVDGVEVHNAKTKPREEQYNSTYRVVSVL |
| | TVLHQDWLNGKEYKCKVSNKALPAPIEKTISKAKGQPREPQVYTL |
| | PPSREEMTKNQVSLTCLVKGFYPSDIAVEWESNGQPENNYKTTPP |
| | VLDSDGSFFLYSKLTVDKSRWQQGNVFSCSVMHEALHNHYTQKS |
| | LSLSPGK |
| Fc Monomer v.3 | HTCPPCPAPEAAGGPSVFLFPPKPKDTLMISRTPEVTCVVVSVSHE |
| (SEQ ID NO: 31) | DPEVKFNWYVDGVEVHNAKTKPREEQYNSTYRVVSVLTVLHQD |
| | WLNGKEYKCKVSNKALPAPIEKTISKAKGQPREPQVYTLPPSREE |
| | MTKNQVSLTCLVKGFYPSDIAVEWESNGQPENNYKTTPPVLDSDG |
| | SFFLYSKLTVDKSRWQQGNVFSCSVMHEALHNHYTQKSLSLSPG |

In some embodiments, the CD40L trimer is connected to the N-terminus of the Fc monomer peptide. In some embodiments, the single chain trimeric CD40L Fc fusion protein comprises any one sequence selected from SEQ ID NOS:1-12, or a fragment thereof.

In one embodiment, the single chain trimeric CD40L Fc fusion protein comprises SEQ ID NO:1. In one embodiment, the single chain trimeric CD40L Fc fusion protein comprises SEQ ID NO:2. In one embodiment, the single chain trimeric CD40L Fc fusion protein comprises SEQ ID NO:3. In one embodiment, the single chain trimeric CD40L Fc fusion protein comprises SEQ ID NO:4. In one embodiment, the single chain trimeric CD40L Fc fusion protein comprises SEQ ID NO:5. In one embodiment, the single chain trimeric CD40L Fc fusion protein comprises SEQ ID NO:6. In one embodiment, the single chain trimeric CD40L Fc fusion protein comprises SEQ ID NO:7. In one embodiment, the single chain trimeric CD40L Fc fusion protein comprises SEQ ID NO:8. In one embodiment, the single chain trimeric CD40L Fc fusion protein comprises SEQ ID NO:9. In one embodiment, the single chain trimeric CD40L Fc fusion protein comprises SEQ ID NO:10. In one embodiment, the single chain trimeric CD40L Fc fusion protein comprises SEQ ID NO:11. In one embodiment, the single chain trimeric CD40L Fc fusion protein comprises SEQ ID NO:12. In one embodiment, the single chain trimeric CD40L Fc fusion protein comprises a fragment of SEQ ID NO:1. In one embodiment, the single chain trimeric CD40L Fc fusion protein comprises a fragment of SEQ ID NO:2. In one embodiment, the single chain trimeric CD40L Fc fusion protein comprises a fragment of SEQ ID NO:3. In one embodiment, the single chain trimeric CD40L Fc fusion protein comprises a fragment of SEQ ID NO:4. In one embodiment, the single chain trimeric CD40L Fc fusion protein comprises a fragment of SEQ ID NO:5. In one embodiment, the single chain trimeric CD40L Fc fusion protein comprises a fragment of SEQ ID NO:6. In one embodiment, the single chain trimeric CD40L Fc fusion protein comprises a fragment of SEQ ID NO:7. In one embodiment, the single chain trimeric CD40L Fc fusion protein comprises a fragment of SEQ ID NO:8. In one embodiment, the single chain trimeric CD40L Fc fusion protein comprises a fragment of SEQ ID NO:9. In one embodiment, the single chain trimeric CD40L Fc fusion protein comprises a fragment of SEQ ID NO:10. In one embodiment, the single chain trimeric CD40L Fc fusion protein comprises a fragment of SEQ ID NO:11. In one embodiment, the single chain trimeric CD40L Fc fusion protein comprises a fragment of SEQ ID NO:12.

In some embodiments, the CD40L trimer is connected to the C-terminus of the Fc monomer peptide. In some embodiments, the single chain trimeric CD40L Fc fusion protein comprises any one sequence selected from SEQ ID NOS:13-19, or a fragment thereof. In one embodiment, the single chain trimeric CD40L Fc fusion protein comprises SEQ ID NO:16 or a fragment thereof. In one embodiment, the single chain trimeric CD40L Fc fusion protein comprises SEQ ID NO:13. In one embodiment, the single chain trimeric CD40L Fc fusion protein comprises SEQ ID NO:14. In one embodiment, the single chain trimeric CD40L Fc fusion protein comprises SEQ ID NO:15. In one embodiment, the single chain trimeric CD40L Fc fusion protein comprises SEQ ID NO:16. In one embodiment, the single chain trimeric CD40L Fc fusion protein comprises SEQ ID NO:17. In one embodiment, the single chain trimeric CD40L Fc fusion protein comprises SEQ ID NO:18. In one embodiment, the single chain trimeric CD40L Fc fusion protein comprises SEQ ID NO: 19. In one embodiment, the single chain trimeric CD40L Fc fusion protein comprises a fragment of SEQ ID NO:13. In one embodiment, the single chain trimeric CD40L Fc fusion protein comprises a fragment of SEQ ID NO:14. In one embodiment, the single chain trimeric CD40L Fc fusion protein comprises a fragment of SEQ ID NO:15. In one embodiment, the single chain trimeric CD40L Fc fusion protein comprises a fragment of SEQ ID NO:16. In one embodiment, the single chain trimeric CD40L Fc fusion protein comprises a fragment of SEQ ID NO:17. In one embodiment, the single chain trimeric CD40L Fc fusion protein comprises a fragment of SEQ ID NO:18. In one embodiment, the single chain trimeric CD40L Fc fusion protein comprises a fragment of SEQ ID NO:19.

In some embodiments, the CD40 ligand subunits comprise a portion of the CD40L extracellular sequence. In some embodiments, the CD40L subunits comprise any one of the sequences selected from SEQ ID NOS:20-22, or a fragment thereof. In one embodiment, the CD40L subunits comprises SEQ ID NO:20. In one embodiment, the CD40L subunits comprises SEQ ID NO:21. In one embodiment, the CD40L subunits comprises SEQ ID NO:22. In one embodiment, the CD40L subunits comprises a fragment of SEQ ID NO:20. In one embodiment, the CD40L subunits comprises a fragment of SEQ ID NO:21. In one embodiment, the CD40L subunits comprises a fragment of SEQ ID NO:22.

In some instances, two single chain trimeric CD40L Fc fusion proteins can form a homodimeric Fc fusion protein or a heterodimeric Fc fusion protein with the latter being preferred. In one embodiment, two single chain trimeric CD40L Fc fusion proteins form a homodimeric Fc fusion protein. In one embodiment, two single chain trimeric CD40L Fc fusion proteins form a heterodimeric Fc fusion protein. In some cases, one monomer of the heterodimeric Fc fusion protein comprises only an Fc monomer peptide or fragment thereof and the other monomer is a single chain trimeric CD40L Fc fusion protein. In one embodiment, one monomer of the heterodimeric Fc fusion protein comprises only an Fc monomer peptide and the other monomer is a single chain trimeric CD40L Fc fusion protein. In one embodiment, one monomer of the heterodimeric Fc fusion protein comprises only a fragment of a Fc monomer peptide and the other monomer is a single chain trimeric CD40L Fc fusion protein. In some embodiments, single chain trimeric CD40L Fc fusion proteins include a variant Fc monomer with one or more amino acid substitutions compared to a reference or wild-type Fc monomer. In some embodiments, one monomer of the heterodimeric Fc fusion protein comprises an Fc monomer peptide or fragment thereof and a protein domain other than CD40L, such as a receptor, ligand or other binding partner. In one embodiment, one monomer of the heterodimeric Fc fusion protein comprises an Fc monomer peptide and a protein domain other than CD40L. In one embodiment, one monomer of the heterodimeric Fc fusion protein comprises a fragment of Fc monomer peptide and a protein domain other than CD40L. In one embodiment, the protein domain other than CD40L is a receptor. In one embodiment, the protein domain other than CD40L is a ligand. In one embodiment, the protein domain other than CD40L is another binding partner. Accordingly, a single chain trimeric CD40L Fc fusion protein can be a component of a heterodimeric Fc fusion protein that is a bispecific molecule. Thus, in some embodiments, provided is a bispecific molecule that comprises a single chain trimeric CD40L Fc fusion protein provided herein.

In some embodiments, an CD40L trimer provided herein is recombinantly fused or chemically conjugated (covalent or non-covalent conjugations) to a target antigen. In one embodiment, an CD40L trimer provided herein is recombinantly fused to a target antigen. In one embodiment, an CD40L trimer provided herein is chemically conjugated to a target antigen. In one embodiment, the chemical conjugation is a covalent conjugation. In one embodiment, the chemical conjugation is a non-covalent conjugation. In some embodiments, the target antigen is recombinantly fused to the C-terminus of the CD40L trimer. In other embodiments, the target antigen is recombinantly fused to the N-terminus of the CD40L trimer. In some embodiments, the CD40L trimer and the target antigen is covalently linked via a peptide tether.

In specific embodiments, provided herein is a single chain trimeric CD40L-target antigen fusion protein comprising (a) three CD40L subunits covalently linked to one another by peptide linkers; and (b) a target antigen.

In some embodiments, the target antigen is for eliciting an immune response. In some embodiments, the target antigen is originated or derived from a pathogen or a diseased cell. As used herein, a target antigen “originated” from a pathogen or a diseased cell refers to a native antigen that is produced by the pathogen or the diseased cell under conditions naturally existing or artificially created. Examples of antigens originated from a pathogen or diseased cell include but are not limited to native proteins expressed by the pathogen or diseased cell in vivo or in vitro. As used herein, a target antigen “derived” from a pathogen or a diseased cell refers to a variant of the native antigen originated therefrom. For example, a variant of a protein or peptide may result from one or more (such as, for example, about 1 to about 25, about 1 to about 20, about 1 to about 15, about 1 to about 10, or about 1 to about 5) changes to an amino acid sequence of the native or unmodified sequence of such protein or peptide. Variants of a native protein may be naturally occurring (such as allelic or splice variants, or fragmented or processed by an immune cell), or may be artificially constructed. In some embodiments, the target antigen is originated from a pathogen. In some embodiments, the target antigen is originated from a diseased cell. In some embodiments, the target antigen is derived from a pathogen. In some embodiments, the target antigen is derived from a diseased cell.

In some embodiments, the target antigen is originated or derived from a protein expressed by an infective pathogen, such as a virus, a bacterial, a fungus or a parasite. In one embodiment, the target antigen is originated from a protein expressed by an infective pathogen. In one embodiment, the target antigen is derived from a protein expressed by an infective pathogen. In one embodiment, the pathogen is a virus. In another embodiment, the pathogen is a bacteria. In one embodiment, the pathogen is a fungus. In another embodiment, the pathogen is a parasite.

In some embodiments, the target antigen is originated or derived from a protein expressed by a diseased host cell that is infected by the infective pathogen. In one embodiment, the target antigen is originated from a protein expressed by a diseased host cell. In one embodiment, the target antigen is derived from a protein expressed by a diseased host cell. In some embodiments, the diseased host cell is infected by an infective pathogen. In one embodiment, the pathogen is a virus. In another embodiment, the pathogen is a bacteria. In one embodiment, the pathogen is a fungus. In another embodiment, the pathogen is a parasite.

In some embodiments, the target antigen is originated or derived from a protein produced by an infective pathogen. In some embodiments, the target antigen is originated from a protein produced by an infective pathogen. In some embodiments, the target antigen is derived from a protein produced by an infective pathogen. In some embodiments, the infective pathogen mediates host entry by the pathogen. In one embodiment, the pathogen is a virus. In another embodiment, the pathogen is a bacteria. In one embodiment, the pathogen is a fungus. In another embodiment, the pathogen is a parasite.

In some embodiments, the target antigen is originated or derived from a protein produced by a cancer cell or a tumor stromal cell. In some embodiments, the target antigen is originated from a protein produced by a cancer cell. In some embodiments, the target antigen is originated from a protein produced by a tumor stromal cell. In some embodiments, the target antigen is derived from a protein produced by a cancer cell. In some embodiments, the target antigen is derived from a protein produced by a tumor stromal cell.

In specific embodiments, the target antigen is a tumor associated antigen (TAA), or an antigenic fragment thereof. In one embodiment, the target antigen is a TAA. In one embodiment, the target antigen is an antigenic fragment of a TAA.

In some embodiments, the target antigen is recognized by the innate immune system of a subject. In some embodiments, the target antigen is processed and presented by antigen presenting cells. In some embodiments, the target antigen is processed and presented by antigen presenting cells in vitro. In some embodiments, the target antigen is processed and presented by antigen presenting cells in vivo. In some embodiments, the antigen presenting cells are B cells, dendritic cells, macrophages, natural killer cells, monocytes, granulocytes, eosinophils, neutrophil, or a combination thereof. In some embodiments, the antigen presenting cells are dendritic cells. In some embodiments, the antigen presenting cells are B cells. In some embodiments, the antigen presenting cells are dendritic cells. In some embodiments, the antigen presenting cells are macrophages. In some embodiments, the antigen presenting cells are natural killer cells. In some embodiments, the antigen presenting cells are monocytes. In some embodiments, the antigen presenting cells are granulocytes. In some embodiments, the antigen presenting cells are eosinophils. In some embodiments, the antigen presenting cells are neutrophils.

In some embodiments, the target antigen is derived from a protein expressed on the surface of a cell, where the expression marks the cell for apoptosis (e.g., via cell-mediated cytotoxicity or antibody-dependent cellular toxicity).

In some embodiments, the target antigen is derived from a protein expressed on the surface of a cell. In certain embodiments, the protein that is marked on the surface of a cell marks the cell for apoptosis. In some embodiments, the apoptosis is via cell-mediated cytotoxicity. In other embodiments, the apoptosis is via antibody-dependent cellular toxicity.

In some embodiments, the target antigen is derived from a protein expressed on the surface of a cell, wherein the expression marks the cell for phagocytosis. In some embodiments, the target antigen is derived from a protein specifically recognized by circulating antibodies in a subject.

In some embodiments, the single chain trimeric CD40L fusion protein can also be chemically conjugated (covalent or non-covalent conjugations) or recombinantly fused, e.g., to a diagnostic agent or detectable agent. In one embodiment, the single chain trimeric CD40L fusion protein is chemically conjugated to another molecule. In one embodiment, the conjugation is a covalent conjugation. In another embodiment, the conjugation is a non-covalent conjugation. In one embodiment, the single chain trimeric CD40L fusion protein is recombinantly fused to another molecule. In some embodiments the other molecule is a diagnostic agent. In some embodiments the other molecule is a detectable agent. In particular embodiments, the single chain trimeric CD40L fusion protein of the present disclosure is coupled with detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

In some embodiments, fusion proteins provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use ofRadiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

In some embodiments, a polypeptide comprising the single chain trimeric CD40L fusion protein provided herein can modulate CD40 upon binding to CD40. In certain embodiments, a polypeptide comprising the single chain trimeric CD40L fusion protein provided herein can activate CD40 upon binding to CD40.

In one aspect, the CD40 is activated upon binding the single chain trimeric CD40L fusion protein. In some embodiments, the single chain trimeric CD40L fusion protein activates CD40 with an EC₅₀ of less than about 4 nM. In some embodiments, the single chain trimeric CD40L fusion protein activates CD40 with an EC₅₀ of less than about 3 nM. In some embodiments, the single chain trimeric CD40L fusion protein activates CD40 with an EC₅₀ of less than about 1 nM. In some embodiments, the single chain trimeric CD40L fusion protein activates CD40 with an EC₅₀ of less than about 500 pM. In some embodiments, the single chain trimeric CD40L fusion protein activates CD40 with an EC₅₀ of less than about 100 pM.

In one aspect, the CD40 is activated upon binding the single chain trimeric CD40L Fc fusion protein. In some embodiments, the single chain trimeric CD40L Fc fusion protein activates CD40 with an EC₅₀ of less than about 4 nM. In some embodiments, the single chain trimeric CD40L Fc fusion protein activates CD40 with an EC₅₀ of less than about 3 nM. In some embodiments, the single chain trimeric CD40L Fc fusion protein activates CD40 with an EC₅₀ of less than about 1 nM. In some embodiments, the single chain trimeric CD40L Fc fusion protein activates CD40 with an EC₅₀ of less than about 500 pM. In some embodiments, the single chain trimeric CD40L Fc fusion protein activates CD40 with an EC₅₀ of less than about 100 pM.

In certain embodiments, the EC₅₀ is less than about 1 nM. In one embodiment, the EC₅₀ is less than about 0.9 nM. In one embodiment, the EC₅₀ is less than about 0.8 nM. In one embodiment, the EC₅₀ is less than about 0.7 nM. In one embodiment, the EC₅₀ is less than about 0.6 nM. In one embodiment, the EC₅₀ is less than about 0.5 nM. In one embodiment, the EC₅₀ is less than about 0.4 nM. In one embodiment, the EC₅₀ is less than about 0.300 nM. In one embodiment, the EC₅₀ is less than about 0.2 nM. In one embodiment, the EC₅₀ is less than about 0.19 nM. In one embodiment, the EC₅₀ is less than about 0.18 nM. In one embodiment, the EC₅₀ is less than about 0.17 nM. In one embodiment, the EC₅₀ is less than about 0.16 nM. In one embodiment, the EC₅₀ is less than about 0.15 nM. In one embodiment, the EC₅₀ is less than about 0.14 nM. In one embodiment, the EC₅₀ is less than about 0.13 nM. In one embodiment, the EC₅₀ is less than about 0.12 nM. In one embodiment, the EC₅₀ is less than about 0.11 nM. In one embodiment, the EC₅₀ is less than about 0.1 nM. In one embodiment, the EC₅₀ is less than about 0.09 nM. In one embodiment, the EC₅₀ is less than about 0.08 nM. In one embodiment, the EC₅₀ is less than about 0.07 nM. In one embodiment, the EC₅₀ is less than about 0.06 nM. In one embodiment, the EC₅₀ is less than about 0.05 nM. In one embodiment, the EC₅₀ is less than about 0.04 nM. In one embodiment, the EC₅₀ is less than about 0.03 nM. In one embodiment, the EC₅₀ is less than about 0.02 nM. In one embodiment, the EC₅₀ is or less than about 0.01 nM. In certain embodiments. In one embodiment, the EC₅₀ is the EC₅₀ is less than about 1 pM. In one embodiment, the EC₅₀ is less than about 0.9 pM. In one embodiment, the EC₅₀ is less than about 0.8 pM. In one embodiment, the EC₅₀ is less than about 0.7 pM. In one embodiment, the EC₅₀ is less than about 0.6 pM. In one embodiment, the EC₅₀ is less than about 0.5 pM. In one embodiment, the EC₅₀ is less than about 0.4 pM. In one embodiment, the EC₅₀ is less than about 0.300 pM. In one embodiment, the EC₅₀ is less than about 0.2 pM. In one embodiment, the EC₅₀ is less than about 0.19 pM. In one embodiment, the EC₅₀ is less than about 0.18 pM. In one embodiment, the EC₅₀ is less than about 0.17 pM. In one embodiment, the EC₅₀ is less than about 0.16 pM. In one embodiment, the EC₅₀ is less than about 0.15 pM. In one embodiment, the EC₅₀ is less than about 0.14 pM. In one embodiment, the EC₅₀ is less than about 0.13 pM. In one embodiment, the EC₅₀ is less than about 0.12 pM. In one embodiment, the EC₅₀ is less than about 0.11 pM. In one embodiment, the EC₅₀ is less than about 0.1 pM. In one embodiment, the EC₅₀ is less than about 0.09 pM. In one embodiment, the EC₅₀ is less than about 0.08 pM. In one embodiment, the EC₅₀ is less than about 0.07 pM. In one embodiment, the EC₅₀ is less than about 0.06 pM. In one embodiment, the EC₅₀ is less than about 0.05 pM. In one embodiment, the EC₅₀ is less than about 0.04 pM. In one embodiment, the EC₅₀ is less than about 0.03 pM. In one embodiment, the EC₅₀ is less than about 0.02 pM. In one embodiment, the EC₅₀ is or less than about 0.01 pM. In certain embodiments. In one embodiment, the EC₅₀ is less than about 1000 pM. In one embodiment, the EC₅₀ is less than about 900 pM. In one embodiment, the EC₅₀ is less than about 800 pM. In one embodiment, the EC₅₀ is less than about 700 pM. In one embodiment, the EC₅₀ is less than about 600 pM. In one embodiment, the EC₅₀ is less than about 500 pM. In one embodiment, the EC₅₀ is less than about 400 pM. In one embodiment, the EC₅₀ is less than about 300 pM. In one embodiment, the EC₅₀ is less than about 200 pM. In one embodiment, the EC₅₀ is less than about 190 pM. In one embodiment, the EC₅₀ is less than about 180 pM. In one embodiment, the EC₅₀ is less than about 170 pM. In one embodiment, the EC₅₀ is less than about 160 pM. In one embodiment, the EC₅₀ is less than about 150 pM. In one embodiment, the EC₅₀ is less than about 140 pM. In one embodiment, the EC₅₀ is less than about 130 pM. In one embodiment, the EC₅₀ is less than about 120 pM. In one embodiment, the EC₅₀ is less than about 110 pM. In one embodiment, the EC₅₀ is less than about 100 pM. In one embodiment, the EC₅₀ is less than about 90 pM. In one embodiment, the EC₅₀ is less than about 80 pM. In one embodiment, the EC₅₀ is less than about 70 pM. In one embodiment, the EC₅₀ is less than about 60 pM. In one embodiment, the EC₅₀ is less than about 50 pM. In one embodiment, the EC₅₀ is less than about 40 pM. In one embodiment, the EC₅₀ is less than about 30 pM. In one embodiment, the EC₅₀ is less than about 20 pM. In one embodiment, the EC₅₀ is less than about 10 pM.

Also provided herein are methods of modulating a CD40 polypeptide comprising contacting the CD40 polypeptide with a polypeptide comprising the single chain trimeric CD40L fusion protein provided herein. Also provided herein are methods of activating a CD40 polypeptide comprising contacting the CD40 polypeptide with a polypeptide comprising the single chain trimeric CD40L fusion protein provided herein.

Also provided are methods of activating a CD40 polypeptide comprising contacting the CD40 polypeptide with a single chain trimeric CD40L Fc fusion protein comprising (a) three CD40L subunits covalently linked to one another by peptide linkers; and (b) an Fc monomer peptide, wherein said single chain trimeric CD40L Fc fusion protein activates the CD40 polypeptide upon binding.

Also provided are method of activating a target cell expressing a CD40 polypeptide comprising contacting the target cell with a polypeptide comprising the single chain trimeric CD40L fusion protein according to the present disclosure, thereby producing an activated target cell. In specific embodiments, the polypeptide contacted with the target cell is a single chain trimeric CD40L Fc fusion protein comprising (a) three CD40L subunits covalently linked to one another by peptide linkers; and (b) an Fc monomer peptide, wherein the polypeptide activates the target cell upon binding CD40. In some embodiments, the activated target cell in turn activates another cell. In some embodiments, the activated target cell activates a T cell. In some embodiments, the activated target cell activates a population of T cells.

In some embodiments, the target cell is an antigen presenting cell. In some embodiments, the target cell is an immune cell. In some embodiments, the target cell is B cells, natural killer cells, dendritic cells, macrophages, monocytes, granulocytes, eosinophils, neutrophils, or a combination thereof. In one embodiment, the target cell is a dendritic cell. In one embodiment, the target cell is a macrophage. In one embodiment, the target cell is a natural killer cell. In one embodiment, the target cell is a monocyte. In one embodiment, the target cell is a granulocyte. In one embodiment, the target cell is an eosinophil. In one embodiment, the target cell is a granulocyte. In one embodiment, the target cell is a neutrophil. In some embodiments the target cell is a Langerhans cell. In some embodiments the target cell is a prostatic glandular cell. In some embodiments the target cell is a B cell. In some embodiments the target cell is a naïve B cell. In some embodiments the target cell is a memory B cell. In some embodiments the target cell is a basal respiratory cell.

In some embodiments, the activated target cell activates another cell. In some embodiments, the activated target cell activates another cell that does not express the CD40 polypeptide. In some embodiments, the activated target cell activates an immune cell. In some embodiments, the activated target cell activates a T cell. In some embodiments, the activated target cell activates CD4+ T cells, CD8+ T cells, MAIT, natural killer cells, neutrophils, or a combination thereof. In one embodiment, the activated target cell activates a CD4+ T cell. In one embodiment, the activated target cell activates a CD8+ T cell. In one embodiment, the activated target cell activates a MAIT. In one embodiment, the activated target cell activates a natural killer cell. In one embodiment, the activated target cell activates a neutrophil.

In some embodiments, activation of the target cell is measured as increased proliferation or maturation of the target cell. In one embodiment, activation of the target cell is measured as increased proliferation of the target cell. In one embodiment, activation of the target cell is measured as increased maturation of the target cell. In particular embodiments, proliferation or maturation of the target cell is increased by about 10%, 20%, 30%, 40%, 45%, 50%, 55%, 60%, 65%, 70%, 75%, 80%, 85%, 90%, 95%, 100%, 125%, 150%, 175%, 200%, 250%, 300%, 400%, 500%, 600%, 700%, 800%, 900% or 1000%. In one embodiment, proliferation of the target cell is increased by about 10-100%. In another embodiment, proliferation of the target cell is increased by about 100-200%. In another embodiment, proliferation of the target cell is increased by about 200-300%. In another embodiment, proliferation of the target cell is increased by about 300-400%. In another embodiment, proliferation of the target cell is increased by about 400-500%. In another embodiment, proliferation of the target cell is increased by about 500-600%. In another embodiment, proliferation of the target cell is increased by about 600-700%. In another embodiment, proliferation of the target cell is increased by about 700-800%. In another embodiment, proliferation of the target cell is increased by about 800-900%. In another embodiment, proliferation of the target cell is increased by about 900-1000%. In one embodiment, proliferation of the target cell is increased by about 10%. In one embodiment, proliferation of the target cell is increased by about 20%. In one embodiment, proliferation of the target cell is increased by about 30%. In one embodiment, proliferation of the target cell is increased by about 40%. In one embodiment, proliferation of the target cell is increased by about 45%. In one embodiment, proliferation of the target cell is increased by about 50%. In one embodiment, proliferation of the target cell is increased by about 55%. In one embodiment, proliferation of the target cell is increased by about 60%. In one embodiment, proliferation of the target cell is increased by about 65%. In one embodiment, proliferation of the target cell is increased by about 70%. In one embodiment, proliferation of the target cell is increased by about 75%. In one embodiment, proliferation of the target cell is increased by about 80%. In one embodiment, proliferation of the target cell is increased by about 85%. In one embodiment, proliferation of the target cell is increased by about 90%. In one embodiment, proliferation of the target cell is increased by about 95%. In one embodiment, proliferation of the target cell is increased by about 100%. In one embodiment, proliferation of the target cell is increased by about 125%. In one embodiment, proliferation of the target cell is increased by about 150%. In one embodiment, proliferation of the target cell is increased by about 175%. In one embodiment, proliferation of the target cell is increased by about 200%. In one embodiment, proliferation of the target cell is increased by about 250%. In one embodiment, proliferation of the target cell is increased by about 300%. In one embodiment, proliferation of the target cell is increased by about 400%. In one embodiment, proliferation of the target cell is increased by about 500%. In one embodiment, proliferation of the target cell is increased by about 600%. In one embodiment, proliferation of the target cell is increased by about 700%. In one embodiment, proliferation of the target cell is increased by about 800%. In one embodiment, proliferation of the target cell is increased by about 900%. In one embodiment, proliferation of the target cell is increased by about 1000%. In one embodiment, maturation of the target cell is increased by about 10-100%. In another embodiment, maturation of the target cell is increased by about 100-200%. In another embodiment, maturation of the target cell is increased by about 200-300%. In another embodiment, maturation of the target cell is increased by about 300-400%. In another embodiment, maturation of the target cell is increased by about 400-500%. In another embodiment, maturation of the target cell is increased by about 500-600%. In another embodiment, maturation of the target cell is increased by about 600-700%. In another embodiment, maturation of the target cell is increased by about 700-800%. In another embodiment, maturation of the target cell is increased by about 800-900%. In another embodiment, maturation of the target cell is increased by about 900-1000%. In one embodiment, maturation of the target cell is increased by about 10%. In one embodiment, maturation of the target cell is increased by about 20%. In one embodiment, maturation of the target cell is increased by about 30%. In one embodiment, maturation of the target cell is increased by about 40%. In one embodiment, maturation of the target cell is increased by about 45%. In one embodiment, maturation of the target cell is increased by about 50%. In one embodiment, maturation of the target cell is increased by about 55%. In one embodiment, maturation of the target cell is increased by about 60%. In one embodiment, maturation of the target cell is increased by about 65%. In one embodiment, maturation of the target cell is increased by about 70%. In one embodiment, maturation of the target cell is increased by about 75%. In one embodiment, maturation of the target cell is increased by about 80%. In one embodiment, maturation of the target cell is increased by about 85%. In one embodiment, maturation of the target cell is increased by about 90%. In one embodiment, maturation of the target cell is increased by about 95%. In one embodiment, maturation of the target cell is increased by about 100%. In one embodiment, maturation of the target cell is increased by about 125%. In one embodiment, maturation of the target cell is increased by about 150%. In one embodiment, maturation of the target cell is increased by about 175%. In one embodiment, maturation of the target cell is increased by about 200%. In one embodiment, maturation of the target cell is increased by about 250%. In one embodiment, maturation of the target cell is increased by about 300%. In one embodiment, maturation of the target cell is increased by about 400%. In one embodiment, maturation of the target cell is increased by about 500%. In one embodiment, maturation of the target cell is increased by about 600%. In one embodiment, maturation of the target cell is increased by about 700%. In one embodiment, maturation of the target cell is increased by about 800%. In one embodiment, maturation of the target cell is increased by about 900%. In one embodiment, maturation of the target cell is increased by about 1000%.

In some embodiments, activation of the target cell is measured as prolonged survival time of the target cell. In particular embodiments, survival time of the target cell is increased by about 10%, 20%, 30%, 40%, 45%, 50%, 55%, 60%, 65%, 70%, 75%, 80%, 85%, 90%, 95%, 100%, 125%, 150%, 175%, 200%, 250%, 300%, 400%, 500%, 600%, 700%, 800%, 900% or 1000%. In one embodiment, survival time of the target cell is increased by about 10-100%. In another embodiment, survival time of the target cell is increased by about 100-200%. In another embodiment, survival time of the target cell is increased by about 200-300%. In another embodiment, survival time of the target cell is increased by about 300-400%. In another embodiment, survival time of the target cell is increased by about 400-500%. In another embodiment, survival time of the target cell is increased by about 500-600%. In another embodiment, survival time of the target cell is increased by about 600-700%. In another embodiment, survival time of the target cell is increased by about 700-800%. In another embodiment, survival time of the target cell is increased by about 800-900%. In another embodiment, survival time of the target cell is increased by about 900-1000%. In one embodiment, survival time of the target cell is increased by about 10%. In one embodiment, survival time of the target cell is increased by about 20%. In one embodiment, survival time of the target cell is increased by about 30%. In one embodiment, survival time of the target cell is increased by about 40%. In one embodiment, survival time of the target cell is increased by about 45%. In one embodiment, survival time of the target cell is increased by about 50%. In one embodiment, survival time of the target cell is increased by about 55%. In one embodiment, survival time of the target cell is increased by about 60%. In one embodiment, survival time of the target cell is increased by about 65%. In one embodiment, survival time of the target cell is increased by about 70%. In one embodiment, survival time of the target cell is increased by about 75%. In one embodiment, survival time of the target cell is increased by about 80%. In one embodiment, survival time of the target cell is increased by about 85%. In one embodiment, survival time of the target cell is increased by about 90%. In one embodiment, survival time of the target cell is increased by about 95%. In one embodiment, survival time of the target cell is increased by about 100%. In one embodiment, survival time of the target cell is increased by about 125%. In one embodiment, survival time of the target cell is increased by about 150%. In one embodiment, survival time of the target cell is increased by about 175%. In one embodiment, survival time of the target cell is increased by about 200%. In one embodiment, survival time of the target cell is increased by about 250%. In one embodiment, survival time of the target cell is increased by about 300%. In one embodiment, survival time of the target cell is increased by about 400%. In one embodiment, survival time of the target cell is increased by about 500%. In one embodiment, survival time of the target cell is increased by about 600%. In one embodiment, survival time of the target cell is increased by about 700%. In one embodiment, survival time of the target cell is increased by about 800%. In one embodiment, survival time of the target cell is increased by about 900%. In one embodiment, survival time of the target cell is increased by about 1000%.

Also provided are methods of activating a T cell comprising contacting the T cell with an antigen presenting cell in the presence of a single chain trimeric CD40L Fc fusion protein comprising (a) three CD40L subunits covalently linked to one another by peptide linkers; and (b) an Fc monomer peptide, wherein the antigen presenting cell expresses a CD40 polypeptide, wherein said single chain trimeric CD40L Fc fusion protein activates the T-cell upon binding the CD40 polypeptide. In some embodiments, the antigen presenting cell presents an antigen to the T cell.

In some embodiments, activation of a population of T cells is measured by increased secretion of pro-inflammatory cytokines by the T cells. In some embodiments, the pro-inflammatory cytokines are selected from IL-1, IL-2, IL-6, IL-12, IL-17, IL-22, IL-23, GM-CSF, TNF-α, IFN-γ or any combination thereof. In one embodiment, the cytokine is IL-1. In one embodiment, the cytokine is IL-2. In one embodiment, the cytokine is IL-6. In one embodiment, the cytokine is IL-12. In one embodiment, the cytokine is IL-17. In one embodiment, the cytokine is IL-22. In one embodiment, the cytokine is IL-23. In one embodiment, the cytokine is GM-CSF. In one embodiment, the cytokine is TNF-α. In one embodiment, the cytokine is IFN-γ.

In some embodiments, the antigen presenting cell presents an antigen to the T cell. In some embodiments, upon activation of the antigen presenting cell by the single chain trimeric CD40L Fc fusion protein, presentation of the antigen by the antigen presenting cell to the T cell is increased. In some embodiments, the population of antigen presenting cells comprises B cells, dendritic cells, macrophages, natural killer cells, monocytes, granulocytes, eosinophils, neutrophils, or a combination thereof. In some embodiments, the population of antigen presenting cells comprise B cells. In some embodiments, the population of antigen presenting cells comprise macrophages. In some embodiments, the population of antigen presenting cells comprise dendritic cells. In some embodiments, the population of antigen presenting cells comprise natural killer cells. In some embodiments, the population of antigen presenting cells comprise monocytes. In some embodiments, the population of antigen presenting cells comprise granulocytes. In some embodiments, the population of antigen presenting cells comprise eosinophils. In some embodiments, the population of antigen presenting cells comprise neutrophils.

Also provided are methods of activating a dendritic cell comprising contacting a CD40 polypeptide with a single chain trimeric CD40L Fc fusion protein comprising (a) three CD40L subunits covalently linked to one another by peptide linkers; and (b) an Fc monomer peptide, wherein said single chain trimeric CD40L Fc fusion protein activates the dendritic cell polypeptide upon binding CD40.

In some embodiments, the methods comprise contacting the CD40 polypeptide with a dimer comprising two single chain trimeric CD40L Fc fusion proteins, each comprising (a) three CD40L subunits covalently linked to one another by peptide linkers; and (b) an Fc monomer peptide.

In some embodiments, provided herein is a method for promoting antibody production by a population of B cells, comprising contacting the B cells with a polypeptide comprising the single chain trimeric CD40L fusion protein according to the present disclosure. In specific embodiments, the polypeptide contacted with the population of B cells is a single chain trimeric CD40L Fc fusion protein comprising (a) three CD40L subunits covalently linked to one another by peptide linkers; and (b) an Fc monomer peptide.

In some embodiments, the B cells are contacted with the polypeptide comprising the single chain trimeric CD40L fusion protein in the presence of a target antigen, and wherein the antibody produced by the B cells specifically binds to the antigen. In some embodiments, the target antigen is chemically conjugated or recombinantly fused to the single chain trimeric CD40L fusion protein. In some embodiments, the target antigen is presented by an antigen-presenting cell. In some embodiments, the target antigen is associated with an MHC class I complex. In some embodiments, the target antigen is associated with an MHC class II complex. In some embodiments, the target antigen is originated or derived from an infectious pathogen. In some embodiments, the target antigen is originated or derived from a diseased cell. In some embodiments, the diseased cell is a cancer cell. In some embodiments, the diseased cell is a cell infected by an infectious pathogen. In some embodiments, the infectious pathogen is a virus, a bacterial, a fungus or a parasite.

In some embodiments, antibody production by the population of B cells is increased by about 10%, 20%, 30%, 40%, 45%, 50%, 55%, 60%, 65%, 70%, 75%, 80%, 85%, 90%, 95%, 100%, 125%, 150%, 175%, 200%, 250%, 300%, 400%, 500%, 600%, 700%, 800%, 900% or 1000%. In one embodiment, antibody production by a population of B cells is increased by about 10-100%. In another embodiment, antibody production by a population of B cells is increased by about 100-200%. In another embodiment, antibody production by a population of B cells is increased by about 200-300%. In another embodiment, antibody production by a population of B cells is increased by about 300-400%. In another embodiment, antibody production by a population of B cells is increased by about 400-500%. In another embodiment, antibody production by a population of B cells is increased by about 500-600%. In another embodiment, antibody production by a population of B cells is increased by about 600-700%. In another embodiment, antibody production by a population of B cells is increased by about 700-800%. In another embodiment, antibody production by a population of B cells is increased by about 800-900%. In another embodiment, antibody production by a population of B cells is increased by about 900-1000%. In one embodiment, antibody production by a population of B cells is increased by about 10%. In one embodiment, antibody production by a population of B cells is increased by about 20%. In one embodiment, antibody production by a population of B cells is increased by about 30%. In one embodiment, antibody production by a population of B cells is increased by about 40%. In one embodiment, antibody production by a population of B cells is increased by about 45%. In one embodiment, antibody production by a population of B cells is increased by about 50%. In one embodiment, antibody production by a population of B cells is increased by about 55%. In one embodiment, antibody production by a population of B cells is increased by about 60%. In one embodiment, antibody production by a population of B cells is increased by about 65%. In one embodiment, antibody production by a population of B cells is increased by about 70%. In one embodiment, antibody production by a population of B cells is increased by about 75%. In one embodiment, antibody production by a population of B cells is increased by about 80%. In one embodiment, antibody production by a population of B cells is increased by about 85%. In one embodiment, antibody production by a population of B cells is increased by about 90%. In one embodiment, antibody production by a population of B cells is increased by about 95%. In one embodiment, antibody production by a population of B cells is increased by about 100%. In one embodiment, antibody production by a population of B cells is increased by about 125%. In one embodiment, antibody production by a population of B cells is increased by about 150%. In one embodiment, antibody production by a population of B cells is increased by about 175%. In one embodiment, antibody production by a population of B cells is increased by about 200%. In one embodiment, antibody production by a population of B cells is increased by about 250%. In one embodiment, antibody production by a population of B cells is increased by about 300%. In one embodiment, antibody production by a population of B cells is increased by about 400%. In one embodiment, antibody production by a population of B cells is increased by about 500%. In one embodiment, antibody production by a population of B cells is increased by about 600%. In one embodiment, antibody production by a population of B cells is increased by about 700%. In one embodiment, antibody production by a population of B cells is increased by about 800%. In one embodiment, antibody production by a population of B cells is increased by about 900%. In one embodiment, antibody production by a population of B cells is increased by about 1000%. In one embodiment, antibody production by a population of B cells is increased by about 10-100%. In another embodiment, antibody production by a population of B cells is increased by about 100-200%. In another embodiment, antibody production by a population of B cells is increased by about 200-300%. In another embodiment, antibody production by a population of B cells is increased by about 300-400%. In another embodiment, antibody production by a population of B cells is increased by about 400-500%. In another embodiment, antibody production by a population of B cells is increased by about 500-600%. In another embodiment, antibody production by a population of B cells is increased by about 600-700%. In another embodiment, antibody production by a population of B cells is increased by about 700-800%. In another embodiment, antibody production by a population of B cells is increased by about 800-900%. In another embodiment, antibody production by a population of B cells is increased by about 900-1000%. In some embodiments, the method further promotes formation of memory B cells capable of producing the antibody in response to the antigen.

In some embodiments, provided herein is a method for increasing secretion of pro-inflammatory cytokines by a population of immune cells, comprising contacting the population of immune cells with a population of antigen presenting cells in the presence of a polypeptide comprising the single chain trimeric CD40L fusion protein according to the present disclosure. In specific embodiments, the polypeptide contacted with the population of antigen presenting cells is a single chain trimeric CD40L Fc fusion protein comprising (a) three CD40L subunits covalently linked to one another by peptide linkers; and (b) an Fc monomer peptide. In some embodiments, the population of antigen presenting cells present an antigen to the population of immune cells. In some embodiments, the population of immune cells comprises T cells. In some embodiments, the population of immune cells is a population of T cells. In specific embodiments, upon activation of the antigen presenting cells, the presentation of the antigen by the population of antigen presenting cells to the population of immune cells is increased. In some embodiments, a minimal percentage of antigen presenting cells presenting the antigen in the population of antigen presenting cells is increased by about 10%, 20%, 30%, 40%, 45%, 50%, 55%, 60%, 65%, 70%, 75%, 80%, 85%, 90%, 95%, or 99%. In one embodiment, a minimal percentage of antigen presenting cells presenting the antigen in the population of antigen presenting cells is increased by about 10-20%. In another embodiment, a minimal percentage of antigen presenting cells presenting the antigen in the population of antigen presenting cells is increased by about 20-30%. In another embodiment, a minimal percentage of antigen presenting cells presenting the antigen in the population of antigen presenting cells is increased by about 30-40%. In another embodiment, a minimal percentage of antigen presenting cells presenting the antigen in the population of antigen presenting cells is increased by about 40-50%. In another embodiment, a minimal percentage of antigen presenting cells presenting the antigen in the population of antigen presenting cells is increased by about 50-60%. In another embodiment, a minimal percentage of antigen presenting cells presenting the antigen in the population of antigen presenting cells is increased by about 60-70%. In another embodiment, a minimal percentage of antigen presenting cells presenting the antigen in the population of antigen presenting cells is increased by about 70-80%. In another embodiment, a minimal percentage of antigen presenting cells presenting the antigen in the population of antigen presenting cells is increased by about 80-90%. In another embodiment, a minimal percentage of antigen presenting cells presenting the antigen in the population of antigen presenting cells is increased by about 90-99%. In some embodiments, a minimal percentage of antigen presenting cells presenting the antigen in the population of antigen presenting cells is increased by about 10%. In some embodiments, a minimal percentage of antigen presenting cells presenting the antigen in the population of antigen presenting cells is increased by about 20%. In some embodiments, a minimal percentage of antigen presenting cells presenting the antigen in the population of antigen presenting cells is increased by about 30%. In some embodiments, a minimal percentage of antigen presenting cells presenting the antigen in the population of antigen presenting cells is increased by about 40%. In some embodiments, a minimal percentage of antigen presenting cells presenting the antigen in the population of antigen presenting cells is increased by about 45%. In some embodiments, a minimal percentage of antigen presenting cells presenting the antigen in the population of antigen presenting cells is increased by about 50%. In some embodiments, a minimal percentage of antigen presenting cells presenting the antigen in the population of antigen presenting cells is increased by about 55%. In some embodiments, a minimal percentage of antigen presenting cells presenting the antigen in the population of antigen presenting cells is increased by about 60%. In some embodiments, a minimal percentage of antigen presenting cells presenting the antigen in the population of antigen presenting cells is increased by about 65%. In some embodiments, a minimal percentage of antigen presenting cells presenting the antigen in the population of antigen presenting cells is increased by about 70%. In some embodiments, a minimal percentage of antigen presenting cells presenting the antigen in the population of antigen presenting cells is increased by about 75%. In some embodiments, a minimal percentage of antigen presenting cells presenting the antigen in the population of antigen presenting cells is increased by about 80%. In some embodiments, a minimal percentage of antigen presenting cells presenting the antigen in the population of antigen presenting cells is increased by about 85%. In some embodiments, a minimal percentage of antigen presenting cells presenting the antigen in the population of antigen presenting cells is increased by about 90%. In some embodiments, a minimal percentage of antigen presenting cells presenting the antigen in the population of antigen presenting cells is increased by about 95%. In some embodiments, a minimal percentage of antigen presenting cells presenting the antigen in the population of antigen presenting cells is increased by about or 99%. In some embodiments, the population of antigen presenting cells comprises B cells, dendritic cells, macrophages, natural killer cells, monocytes, granulocytes, eosinophils, neutrophils, or a combination thereof. In some embodiments, the population of antigen presenting cells comprise B cells. In some embodiments, the population of antigen presenting cells comprise macrophages. In some embodiments, the population of antigen presenting cells comprise dendritic cells. In some embodiments, the population of antigen presenting cells comprise natural killer cells. In some embodiments, the population of antigen presenting cells comprise monocytes. In some embodiments, the population of antigen presenting cells comprise granulocytes. In some embodiments, the population of antigen presenting cells comprise eosinophils. In some embodiments, the population of antigen presenting cells comprise neutrophils. In some embodiments, the cytokine is IL-1, IL-2, IL-6, IL-12, IL-17, IL-22, IL-23, GM-CSF, TNF-α, IFN-γ or any combination thereof. In one embodiment, the cytokine is IL-1. In one embodiment, the cytokine is IL-2. In one embodiment, the cytokine is IL-6. In one embodiment, the cytokine is IL-12. In one embodiment, the cytokine is IL-17. In one embodiment, the cytokine is IL-22. In one embodiment, the cytokine is IL-23. In one embodiment, the cytokine is GM-CSF. In one embodiment, the cytokine is TNF-α. In one embodiment, the cytokine is IFN-γ. In some embodiments, the cytokine production is increased by about 10%, 20%, 30%, 40%, 45%, 50%, 55%, 60%, 65%, 70%, 75%, 80%, 85%, 90%, 95%, 100%, 125%, 150%, 175%, 200%, 250%, 300%, 400%, 500%, 600%, 700%, 800%, 900% or 1000%. In one embodiment, cytokine production is increased by about 10-100%. In another embodiment, cytokine production is increased by about 100-200%. In another embodiment, cytokine production is increased by about 200-300%. In another embodiment, cytokine production is increased by about 300-400%. In another embodiment, cytokine production is increased by about 400-500%. In another embodiment, cytokine production is increased by about 500-600%. In another embodiment, cytokine production is increased by about 600-700%. In another embodiment, cytokine production is increased by about 700-800%. In another embodiment, cytokine production is increased by about 800-900%. In another embodiment, cytokine production is increased by about 900-1000%. In another embodiment, cytokine production is increased by about 10-1000%. In one embodiment, cytokine production is increased by about 20%. In one embodiment, cytokine production is increased by about 30%. In one embodiment, cytokine production is increased by about 40%. In one embodiment, cytokine production is increased by about 45%. In one embodiment, cytokine production is increased by about 50%. In one embodiment, cytokine production is increased by about 55%. In one embodiment, cytokine production is increased by about 60%. In one embodiment, cytokine production is increased by about 65%. In one embodiment, cytokine production is increased by about 70%. In one embodiment, cytokine production is increased by about 75%. In one embodiment, cytokine production is increased by about 80%. In one embodiment, cytokine production is increased by about 85%. In one embodiment, cytokine production is increased by about 90%. In one embodiment, cytokine production is increased by about 95%. In one embodiment, cytokine production is increased by about 100%. In one embodiment, cytokine production is increased by about 125%. In one embodiment, cytokine production is increased by about 150%. In one embodiment, cytokine production is increased by about 175%. In one embodiment, cytokine production is increased by about 200%. In one embodiment, cytokine production is increased by about 250%. In one embodiment, cytokine production is increased by about 300%. In one embodiment, cytokine production is increased by about 400%. In one embodiment, cytokine production is increased by about 500%. In one embodiment, cytokine production is increased by about 600%. In one embodiment, cytokine production is increased by about 700%. In one embodiment, cytokine production is increased by about 800%. In one embodiment, cytokine production is increased by about 900%. In one embodiment, cytokine production is increased by about 1000%.

In some embodiments, provided herein is a method for increasing phagocytosis of diseased cells by a population of macrophages, comprising contacting the diseased cells, the macrophages, or both the diseased cells and the macrophage with a polypeptide comprising the single chain trimeric CD40L fusion protein according to the present disclosure. In specific embodiments, the polypeptide contacted with the population of macrophages is a single chain trimeric CD40L Fc fusion protein comprising (a) three CD40L subunits covalently linked to one another by peptide linkers; and (b) an Fc monomer peptide.

In some embodiments, a minimal percentage of phagocytotic macrophages in the population of macrophages is increased by about 10%, 20%, 30%, 40%, 45%, 50%, 55%, 60%, 65%, 70%, 75%, 80%, 85%, 90%, 95%, or 99%. In one embodiment, a minimal percentage of phagocytotic macrophages in the population of macrophages is increased by about 10-20%. In another embodiment, a minimal percentage of phagocytotic macrophages in the population of macrophages is increased by about 20-30%. In another embodiment, a minimal percentage of phagocytotic macrophages in the population of macrophages is increased by about 30-40%. In another embodiment, a minimal percentage of phagocytotic macrophages in the population of macrophages is increased by about 40-50%. In another embodiment, a minimal percentage of phagocytotic macrophages in the population of macrophages is increased by about 50-60%. In another embodiment, a minimal percentage of phagocytotic macrophages in the population of macrophages is increased by about 60-70%. In another embodiment, a minimal percentage of phagocytotic macrophages in the population of macrophages is increased by about 70-80%. In another embodiment, a minimal percentage of phagocytotic macrophages in the population of macrophages is increased by about 80-90%. In another embodiment, a minimal percentage of phagocytotic macrophages in the population of macrophages is increased by about 90-99%. In some embodiments, a minimal percentage of phagocytotic macrophages in the population of macrophages is increased by about 10%. In some embodiments, a minimal percentage of phagocytotic macrophages in the population of macrophages is increased by about 20%. In some embodiments, a minimal percentage of phagocytotic macrophages in the population of macrophages is increased by about 30%. In some embodiments, a minimal percentage of phagocytotic macrophages in the population of macrophages is increased by about 40%. In some embodiments, a minimal percentage of phagocytotic macrophages in the population of macrophages is increased by about 45%. In some embodiments, a minimal percentage of phagocytotic macrophages in the population of macrophages is increased by about 50%. In some embodiments, a minimal percentage of phagocytotic macrophages in the population of macrophages is increased by about 55%. In some embodiments, a minimal percentage of phagocytotic macrophages in the population of macrophages is increased by about 60%. In some embodiments, a minimal percentage of phagocytotic macrophages in the population of macrophages is increased by about 65%. In some embodiments, a minimal percentage of phagocytotic macrophages in the population of macrophages is increased by about 70%. In some embodiments, a minimal percentage of phagocytotic macrophages in the population of macrophages is increased by about 75%. In some embodiments, a minimal percentage of phagocytotic macrophages in the population of macrophages is increased by about 80%. In some embodiments, a minimal percentage of phagocytotic macrophages in the population of macrophages is increased by about 85%. In some embodiments, a minimal percentage of phagocytotic macrophages in the population of macrophages is increased by about 90%. In some embodiments, a minimal percentage of phagocytotic macrophages in the population of macrophages is increased by about 95%. In some embodiments, a minimal percentage of phagocytotic macrophages in the population of macrophages is increased by about or 99%.

In some embodiments, the phagocytosis by macrophages is measured by co-culturing macrophages labeled with a first fluorescent dye and diseased cells labeled with a second fluorescent dye, wherein the first fluorescent dye and the second fluorescent dye are different. In some embodiments, the percentage of phagocytotic macrophages is measured by determining the percentage of macrophages comprising the diseased cells. In specific embodiments, the diseased cells are cancer cells. In specific embodiments, the diseased cells are cells infected by an infectious pathogen. In some embodiments, the infectious pathogen is a virus, a bacterial, a fungus or a parasite.

In some embodiments, provided herein is a method for increasing antigen presentation by a population of dendritic cells, comprising contacting the dendritic cells with a polypeptide comprising the single chain trimeric CD40L fusion protein according to the present disclosure. In specific embodiments, the polypeptide contacted with the population of dendritic cells is a single chain trimeric CD40L Fc fusion protein comprising (a) three CD40L subunits covalently linked to one another by peptide linkers; and (b) an Fc monomer peptide. In some embodiments, the antigen is originated or derived from an infectious pathogen. In some embodiments, the antigen is originated or derived from a diseased cell. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof. In some embodiments, the antigen is chemically conjugated or recombinantly fused to the polypeptide comprising the single chain trimeric CD40L fusion protein.

In some embodiments, the antigen presentation by the dendritic cells is measured by co-culturing dendritic cells labeled with a first fluorescent dye and the antigen labeled with a second fluorescent dye, wherein the first fluorescent dye and the second fluorescent dye are different. In some embodiments, percentage of dendritic cells presenting the antigen is measured by determining the percentage of dendritic cells co-localizing with the antigen in the population of dendritic cells. In some embodiments, a minimal percentage of dendritic cells presenting the antigen in the population of dendritic cells is increased by about 10%, 20%, 30%, 40%, 45%, 50%, 55%, 60%, 65%, 70%, 75%, 80%, 85%, 90%, 95%, or 99%. In one embodiment, a minimal percentage of dendritic cells presenting the antigen in the population of dendritic cells is increased by about 10-20%. In another embodiment, a minimal percentage of dendritic cells presenting the antigen in the population of dendritic cells is increased by about 20-30%. In another embodiment, a minimal percentage of dendritic cells presenting the antigen in the population of dendritic cells is increased by about 30-40%. In another embodiment, a minimal percentage of dendritic cells presenting the antigen in the population of dendritic cells is increased by about 40-50%. In another embodiment, a minimal percentage of dendritic cells presenting the antigen in the population of dendritic cells is increased by about 50-60%. In another embodiment, a minimal percentage of dendritic cells presenting the antigen in the population of dendritic cells is increased by about 60-70%. In another embodiment, a minimal percentage of dendritic cells presenting the antigen in the population of dendritic cells is increased by about 70-80%. In another embodiment, a minimal percentage of dendritic cells presenting the antigen in the population of dendritic cells is increased by about 80-90%. In another embodiment, a minimal percentage of dendritic cells presenting the antigen in the population of dendritic cells is increased by about 90-99%. In some embodiments, a minimal percentage of dendritic cells presenting the antigen in the population of dendritic cells is increased by about 10%. In some embodiments, a minimal percentage of dendritic cells presenting the antigen in the population of dendritic cells is increased by about 20%. In some embodiments, a minimal percentage of dendritic cells presenting the antigen in the population of dendritic cells is increased by about 30%. In some embodiments, a minimal percentage of dendritic cells presenting the antigen in the population of dendritic cells is increased by about 40%. In some embodiments, a minimal percentage of dendritic cells presenting the antigen in the population of dendritic cells is increased by about 45%. In some embodiments, a minimal percentage of dendritic cells presenting the antigen in the population of dendritic cells is increased by about 50%. In some embodiments, a minimal percentage of dendritic cells presenting the antigen in the population of dendritic cells is increased by about 55%. In some embodiments, a minimal percentage of dendritic cells presenting the antigen in the population of dendritic cells is increased by about 60%. In some embodiments, a minimal percentage of dendritic cells presenting the antigen in the population of dendritic cells is increased by about 65%. In some embodiments, a minimal percentage of dendritic cells presenting the antigen in the population of dendritic cells is increased by about 70%. In some embodiments, a minimal percentage of dendritic cells presenting the antigen in the population of dendritic cells is increased by about 75%. In some embodiments, a minimal percentage of dendritic cells presenting the antigen in the population of dendritic cells is increased by about 80%. In some embodiments, a minimal percentage of dendritic cells presenting the antigen in the population of dendritic cells is increased by about 85%. In some embodiments, a minimal percentage of dendritic cells presenting the antigen in the population of dendritic cells is increased by about 90%. In some embodiments, a minimal percentage of dendritic cells presenting the antigen in the population of dendritic cells is increased by about 95%. In some embodiments, a minimal percentage of dendritic cells presenting the antigen in the population of dendritic cells is increased by about or 99%.

In some embodiments, provided herein is a method for increasing expression of a CD40 polypeptide by a target cell, comprising contacting the target cell with a polypeptide comprising the single chain trimeric CD40L fusion protein according to the present disclosure. In specific embodiments, the polypeptide contacted with the target cell is a single chain trimeric CD40L Fc fusion protein comprising (a) three CD40L subunits covalently linked to one another by peptide linkers; and (b) an Fc monomer peptide.

In some embodiments, the target cell is a diseased cell. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof. In some embodiments, the target cell is an immune cell selected from B cells, natural killer cells, dendritic cells, macrophages, monocytes, granulocytes, eosinophils, neutrophils, or a combination thereof.

In some embodiments, the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99%. In one embodiment, the population of the diseased cells is reduced by about 10-20%. In another embodiment, the population of the diseased cells is reduced by about 20-30%. In another embodiment, the population of the diseased cells is reduced by about 30-40%. In another embodiment, the population of the diseased cells is reduced by about 40-50%. In another embodiment, the population of the diseased cells is reduced by about 50-60%. In another embodiment, the population of the diseased cells is reduced by about 60-70%. In another embodiment, the population of the diseased cells is reduced by about 70-80%. In another embodiment, the population of the diseased cells is reduced by about 80-90%. In another embodiment, the population of the diseased cells is reduced by about 90-99%. In one embodiment, the population of the diseased cells is reduced by about 10%. In one embodiment, the population of the diseased cells is reduced by about 20%. In one embodiment, the population of the diseased cells is reduced by about 30%. In one embodiment, the population of the diseased cells is reduced by about 40%. In one embodiment, the population of the diseased cells is reduced by about 45%. In one embodiment, the population of the diseased cells is reduced by about 50%. In one embodiment, the population of the diseased cells is reduced by about 55%. In one embodiment, the population of the diseased cells is reduced by about 60%. In one embodiment, the population of the diseased cells is reduced by about 65%. In one embodiment, the population of the diseased cells is reduced by about 70%. In one embodiment, the population of the diseased cells is reduced by about 75%. In one embodiment, the population of the diseased cells is reduced by about 80%. In one embodiment, the population of the diseased cells is reduced by about 85%. In one embodiment, the population of the diseased cells is reduced by about 90%. In one embodiment, the population of the diseased cells is reduced by about 95%. In one embodiment, the population of the diseased cells is reduced by about 99%. In one embodiment, the population of the diseased cells is reduced by about 100%.

In some embodiments, provided herein is a method for forming a pro-inflammatory milieu in a tissue surrounding a population of diseased cells, comprising contacting the tissue with a polypeptide comprising the single chain trimeric CD40L fusion protein according to the present disclosure. In specific embodiments, the polypeptide contacted with the tissue is a single chain trimeric CD40L Fc fusion protein comprising (a) three CD40L subunits covalently linked to one another by peptide linkers; and (b) an Fc monomer peptide.

In some embodiments, infiltration of activated B cells, CD4+ T cells, CD8+ T cells, MAITs, dendritic cells, macrophages, natural killer cells, monocytes, granulocytes, eosinophils and/or neutrophils in the tissue is increased. In some embodiments, infiltration of activated B cells is increased. In some embodiments, infiltration of CD4+ T cells is increased. In some embodiments, infiltration of CD8+ T cells is increased. In some embodiments, infiltration of dendritic cells is increased. In some embodiments, infiltration of macrophages is increased. In some embodiments, infiltration of natural killer cells is increased. In some embodiments, infiltration of monocytes is increased. In some embodiments, infiltration of granulocytes is increased. In some embodiments, infiltration of eosinophils is increased. In some embodiments, infiltration of neutrophils is increased.

In some embodiments, concentration of a pro-inflammatory cytokine is increased in the tissue. In some embodiments, the pro-inflammatory cytokine is IL-1, IL-2, IL-6, IL-12, IL-17, IL-22, IL-23, GM-CSF, TNF-α, IFN-γ, or any combination thereof. In one embodiment, the cytokine is IL-1. In one embodiment, the cytokine is IL-2. In one embodiment, the cytokine is IL-6. In one embodiment, the cytokine is IL-12. In one embodiment, the cytokine is IL-17. In one embodiment, the cytokine is IL-22. In one embodiment, the cytokine is IL-23. In one embodiment, the cytokine is GM-CSF. In one embodiment, the cytokine is TNF-α. In one embodiment, the cytokine is IFN-γ.

In some embodiments, presentation of antigens originated or derived from the diseased cells by antigen presentation cells is increased in the tissue. In some embodiments, phagocytosis of the diseased cells is increased in the tissue. In some embodiments, apoptosis of the diseased cells induced by cell-mediated cytotoxicity is increased in the tissue. In some embodiments, apoptosis of the diseased cells induced by antibody-dependent cellular cytotoxicity is increased in the tissue. In some embodiments, the population of the diseased cells is reduced in the tissue. In some embodiments, the population of the diseased cells is reduced by about 10%, 20%, 30%, 40%, 45%, 50%, 55%, 60%, 65%, 70%, 75%, 80%, 85%, 90%, 95%, or 99% in the tissue. In one embodiment, the population of the diseased cells is reduced by about 10-20%. In another embodiment, the population of the diseased cells is reduced by about 20-30%. In another embodiment, the population of the diseased cells is reduced by about 30-40%. In another embodiment, the population of the diseased cells is reduced by about 40-50%. In another embodiment, the population of the diseased cells is reduced by about 50-60%. In another embodiment, the population of the diseased cells is reduced by about 60-70%. In another embodiment, the population of the diseased cells is reduced by about 70-80%. In another embodiment, the population of the diseased cells is reduced by about 80-90%. In another embodiment, the population of the diseased cells is reduced by about 90-99%. In one embodiment, the population of the diseased cells is reduced by about 10%. In one embodiment, the population of the diseased cells is reduced by about 20%. In one embodiment, the population of the diseased cells is reduced by about 30%. In one embodiment, the population of the diseased cells is reduced by about 40%. In one embodiment, the population of the diseased cells is reduced by about 45%. In one embodiment, the population of the diseased cells is reduced by about 50%. In one embodiment, the population of the diseased cells is reduced by about 55%. In one embodiment, the population of the diseased cells is reduced by about 60%. In one embodiment, the population of the diseased cells is reduced by about 65%. In one embodiment, the population of the diseased cells is reduced by about 70%. In one embodiment, the population of the diseased cells is reduced by about 75%. In one embodiment, the population of the diseased cells is reduced by about 80%. In one embodiment, the population of the diseased cells is reduced by about 85%. In one embodiment, the population of the diseased cells is reduced by about 90%. In one embodiment, the population of the diseased cells is reduced by about 95%. In one embodiment, the population of the diseased cells is reduced by about 99%. In one embodiment, the population of the diseased cells is reduced by about 100%.

Methods of Use

According to certain embodiments, the described single chain trimeric CD40L Fc fusion protein can be provided in a buffered composition for storage or use. Suitable buffers for the storage of the described single chain trimeric CD40L Fc fusion protein would serve to maintain the

In one aspect, provided herein is a method of treating a disease.

Also provided herein is a single chain trimeric CD40L Fc fusion protein as described herein for use in therapy. Such therapy includes those in accordance with the methods of treatment and therapeutic uses defined herein and all embodiments thereof.

Also provided herein is a single chain trimeric CD40L Fc fusion protein as described herein for use in the treatment of a disease or disorder. Such method includes those in accordance with the methods of treatment of a disease or disorder defined herein and all embodiments thereof

In some embodiments, provided herein is a method for eliminating a diseased cell in a subject, comprising administering an effective amount of a polypeptide comprising the single chain trimeric CD40L fusion protein according to the present disclosure to the subject. In specific embodiments, the polypeptide administered to the subject is a single chain trimeric CD40L Fc fusion protein comprising (a) three CD40L subunits covalently linked to one another by peptide linkers; and (b) an Fc monomer peptide. In some embodiments, the diseased cell does not express a CD40 polypeptide. In some embodiments, the diseased cell expresses a CD40 polypeptide. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

Also provided herein is a single chain trimeric CD40L Fc fusion protein as described herein for use in the elimination of a diseased cell in a subject. In certain embodiments, the diseased cell is a cancer cell. In other embodiments, the diseased cell is a cell infected with a pathogen.

In some embodiments, provided herein is a method for treating cancer in a subject in need thereof, comprising administering an effective amount of a polypeptide comprising the single chain trimeric CD40L fusion protein according to the present disclosure to the subject. In specific embodiments, the polypeptide administered to the subject is a single chain trimeric CD40L Fc fusion protein comprising (a) three CD40L subunits covalently linked to one another by peptide linkers; and (b) an Fc monomer peptide.

Also provided are methods of treating cancer in a subject comprising administering to the subject a therapeutically effective amount of a dimer comprising two single chain trimeric CD40L Fc fusion proteins, each comprising (a) three CD40L subunits covalently linked to one another by peptide linkers; and (b) an Fc monomer peptide. In some embodiments, the method comprises administering a homodimer. In some embodiments, the method comprises administering a homodimer.

Also provided herein is a polypeptide comprising the single chain trimeric CD40L fusion protein as described herein for use in the treatment of a cancer. Such treatment includes those in accordance with the methods of treating cancer defined herein and all embodiments thereof. In specific embodiments, the polypeptide for use in the treatment of a cancer is a single chain trimeric CD40L Fc fusion protein comprising (a) three CD40L subunits covalently linked to one another by peptide linkers; and (b) an Fc monomer peptide.

Also provided herein is a single chain trimeric CD40L Fc fusion protein as described herein for use in the treatment of a cancer. Such treatment includes those in accordance with the methods of treating cancer defined herein and all embodiments thereof.

Also provided herein is a dimer comprising two single chain trimeric CD40L Fc fusion proteins as described herein for use in the treatment of a cancer. Such treatment includes those in accordance with the methods of treating cancer defined herein and all embodiments thereof.

In some embodiments, the cancer is a solid cancer. In some embodiments, the cancer is a liquid cancer. In some embodiments, the cancer is selected from the group consisting of melanoma, mesothelioma, advanced solid tumor and lymphoma. In one embodiment, the cancer is a melanoma. In one embodiment, the cancer is a mesothelioma. In one embodiment, the cancer is an advanced solid tumor. In one embodiment, the cancer is a lymphoma.

In some embodiments, the method comprises administering a pharmaceutical composition comprising a pharmaceutically acceptable carrier and a single chain trimeric CD40L Fc fusion protein comprising (a) three CD40L subunits covalently linked to one another by peptide linkers; and (b) an Fc monomer peptide. In some embodiments, the method comprises administering a homodimer. In some embodiments, the method comprises administering a heterodimer.

In some embodiments, the method of treating cancer enhances an innate anti-neoplastic immune response. An “anti-neoplastic immune response,” as used herein, refers to immune cells such as, for example, T cells, B cells, or other antigen presenting cells (e.g., dendritic cells (DCs)) of an individual being recruited, primed and/or activated to mount an immune response against a specific tumor target. In some embodiments, antigen presenting cells activated by the polypeptide comprising the single chain trimeric CD40L fusion protein according to the present disclosure can in turn activates additional cell types, such as T-cells. Alternatively, the anti-neoplastic immune response comprises a reduction in tumor burden. The term also encompasses recruitment of cytokines, such as granulocyte macrophage colony stimulating factor (GM-CSF) are to amplify immune activation and/or induce migration of the primed cells to lymph nodes.

In one embodiment, the disease is cancer. In some embodiments, the cancer is selected from the group consisting of melanoma, mesothelioma, advanced solid tumor and lymphoma. In some embodiments, the cancer is a solid tumor cancer. In other embodiments, the cancer is a blood cancer.

In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor, is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hurthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

In one aspect, the subject is a subject in need thereof. In another aspect, the subject is a human.

As used herein, the terms “effective amount” or “therapeutically effective amount” refer to an amount of an active ingredient or component that elicits the desired biological or medicinal response in a subject. The term refers to an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

According to particular embodiments, a therapeutically effective amount refers to the amount of therapy which is sufficient to achieve one or more, two or more, three or more, four or more, or five or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In some embodiments, a single chain trimeric CD40L Fc fusion protein or fragment thereof, as provided herein, is used in combination with a second therapy. In some embodiments, the second therapy is selected from the group consisting of surgery, radiation therapy, chemotherapy, immunotherapy, targeted therapy, hormone therapy, bone marrow transplantation, cryoablation, and radiofrequency ablation.

In some embodiments, the second therapy is immunotherapy. In some embodiments, the immunotherapy encompasses monoclonal antibodies, tumor-agnostic treatments, such as checkpoint inhibitors, T-cell therapy, such as chimeric antigen receptor (CAR) T-cell therapy, or cancer vaccines.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In another aspect, provided herein is a method for treating an infection in a subject in need thereof, comprising administering an effective amount a polypeptide comprising the single chain trimeric CD40L fusion protein according to the present disclosure to the subject. In specific embodiments, the polypeptide administered to the subject is a single chain trimeric CD40L Fc fusion protein comprising (a) three CD40L subunits covalently linked to one another by peptide linkers; and (b) an Fc monomer peptide. In some embodiments, the treatment enhances an innate, humoral, or cell-mediated anti-infective immune response. In one embodiment, the treatment enhances an innate anti-infective immune response. In one embodiment, the treatment enhances a humoral anti-infective immune response. In one embodiment, the treatment enhances a cell-mediated anti-infective immune response.

Also provided herein is a polypeptide comprising the single chain trimeric CD40L fusion protein as described herein for use in the treatment of an infection. Such treatment includes those in accordance with the methods of treating infections defined herein and all embodiments thereof. In specific embodiments, the polypeptide for use in the treatment of an infection is a single chain trimeric CD40L Fc fusion protein comprising (a) three CD40L subunits covalently linked to one another by peptide linkers; and (b) an Fc monomer peptide.

Also provided herein is a single chain trimeric CD40L Fc fusion protein as described herein for use in the treatment of an infection. Such treatment includes those in accordance with the methods for treating an infection defined herein and all embodiments thereof.

Also provided herein is a dimer comprising two single chain trimeric CD40L Fc fusion proteins as described herein for use in the treatment of an infection. Such treatment includes those in accordance with the methods of treating infections defined herein and all embodiments thereof.

In some embodiments the infection is caused by an infective pathogen, such as a virus, a bacterial, a fungus or a parasite. In one embodiment, the pathogen is a virus. In another embodiment, the pathogen is a bacteria. In one embodiment, the pathogen is a fungus. In another embodiment, the pathogen is a parasite. In some embodiments, the polypeptide is co-administered with a vaccine composition for preventing the infection in the subject.

In some embodiments, the polypeptide is co-administered with the vaccine composition simultaneously or sequentially.

In another aspect, provided herein is a method for improving the response of a subject to a vaccine, comprising administering an effective amount a polypeptide comprising the single chain trimeric CD40L fusion protein according to the present disclosure to the subject. In a specific embodiment, the single chain trimeric CD40L fusion protein is administered to the subject simultaneously or sequentially with the vaccine. In specific embodiments, the polypeptide administered to the subject is a single chain trimeric CD40L Fc fusion protein comprising (a) three CD40L subunits covalently linked to one another by peptide linkers; and (b) an Fc monomer peptide. In some embodiments, the treatment enhances an innate, humoral, or cell-mediated immune response to the vaccine. In one embodiment, the treatment enhances an innate immune response to the vaccine. In one embodiment, the treatment enhances a humoral immune response to the vaccine. In one embodiment, the treatment enhances a cell-mediated immune response to the vaccine.

In some embodiments the vaccine is against an infective pathogen, such as a virus, a bacterial, a fungus or a parasite. In one embodiment, the pathogen is a virus. In another embodiment, the pathogen is a bacteria. In one embodiment, the pathogen is a fungus. In another embodiment, the pathogen is a parasite. In another embodiment, the vaccine is against a cancer. In another embodiment, the vaccine is against a tumor. In another embodiment, the vaccine is against an allergen. Other types of vaccines are also contemplated. In some embodiments, the polypeptide is co-administered with a vaccine composition for enhancing the immune response to the vaccine in the subject. In some embodiments, the polypeptide acts as an adjuvant for the vaccine.

In some embodiments, the single chain trimeric CD40L Fc fusion proteins described herein are administered to a subject in need thereof. In some embodiments, the subject is human. In yet other embodiments, the single chain trimeric CD40L Fc fusion protein is administered to the subject via oral delivery, buccal delivery, nasal delivery or inhalation delivery.

In yet other embodiments, provided herein is use of a single chain trimeric CD40L Fc fusion protein provided herein for treating a disease or disorder in subject. In some embodiments, the therapeutic molecule is administered to the subject via oral delivery. In some embodiments, the therapeutic molecule is administered to the subject via buccal delivery. In some embodiments, the therapeutic molecule is administered to the subject via nasal delivery. In some embodiments, the therapeutic molecule is administered to the subject via inhalation delivery.

Also provided is a system comprising a means for providing a single chain trimeric CD40L Fc fusion protein comprising (a) three CD40L subunits covalently linked to one another by peptide linkers; and (b) an Fc monomer peptide.

In another aspect, provided herein is a system comprising a means for providing a dimer comprising two single chain trimeric CD40L Fc fusion protein comprising (a) three CD40L subunits covalently linked to one another by peptide linkers; and (b) an Fc monomer peptide.

In yet another aspect, provided herein is a system comprising a means for providing a polynucleotide encoding a single chain trimeric CD40L Fc fusion protein comprising (a) three CD40L subunits covalently linked to one another by peptide linkers; and (b) an Fc monomer peptide.

In one aspect, provided herein is a system comprising a means for providing a vector comprising a polynucleotide encoding a single chain trimeric CD40L Fc fusion protein comprising (a) three CD40L subunits covalently linked to one another by peptide linkers; and (b) an Fc monomer peptide.

In another aspect, provided herein is a system comprising a host cell comprising a vector comprising a polynucleotide encoding a single chain trimeric CD40L Fc fusion protein comprising (a) three CD40L subunits covalently linked to one another by peptide linkers; and (b) an Fc monomer peptide.

In yet another aspect, provided herein is a system providing a means for providing a pharmaceutical composition comprising a pharmaceutically acceptable carrier and a single chain trimeric CD40L Fc fusion protein comprising (a) three CD40L subunits covalently linked to one another by peptide linkers; and (b) an Fc monomer peptide.

In one aspect, provided herein is a system providing a kit comprising a single chain trimeric CD40L Fc fusion protein comprising (a) three CD40L subunits covalently linked to one another by peptide linkers; and (b) an Fc monomer peptide.

Also provided are nucleic acid molecules encoding single chain trimeric CD40L Fc fusion proteins as well as nucleic acid molecules encoding the components of the single chain trimeric CD40L Fc fusion proteins described herein. Also provided are kits comprising the vector and packaging for the same. Also provided are host cells comprising the vectors containing the nucleic acid molecules encoding the single chain trimeric CD40L Fc fusion proteins described herein.

Also provided is a process for making a single chain trimeric CD40L Fc fusion protein or fragment thereof.

In one aspect, provided herein is a method for producing a single chain trimeric CD40L Fc fusion protein or fragment thereof comprising (a) introducing into a host cell a polynucleotide encoding a single chain trimeric CD40L Fc fusion protein comprising (i) three CD40L subunits covalently linked to one another by peptide linkers; and (ii) an Fc monomer peptide; (b) culturing the host cell under conditions to produce the single chain trimeric CD40L Fc fusion protein or fragment thereof, and (c) recovering the single chain trimeric CD40L Fc fusion protein or fragment thereof from the cell or culture.

In another aspect, provided herein is a method of producing a dimer comprising two single chain trimeric CD40L Fc fusion proteins comprising (a) introducing into a host cell a polynucleotide encoding a single chain trimeric CD40L Fc fusion protein comprising (i) three CD40L subunits covalently linked to one another by peptide linkers; and (ii) an Fc monomer peptide; (b) culturing the host cell under conditions to produce the single chain trimeric CD40L Fc fusion protein or fragment thereof; (c) recovering the single chain trimeric CD40L Fc fusion protein or fragment thereof from the cell or culture, and (d) combining single chain trimeric CD40L Fc fusion proteins or fragments thereof under conditions that favor dimerization.

In yet another aspect, provided herein is a method of producing a pharmaceutical composition of a single chain trimeric CD40L Fc fusion protein or fragment thereof comprising combining the single chain trimeric CD40L Fc fusion protein or fragment thereof with a pharmaceutically acceptable carrier to obtain the pharmaceutical composition.

In yet another aspect, provided herein is a method of producing a vaccine composition comprising a single chain trimeric CD40L Fc fusion protein or fragment thereof comprising combining the single chain trimeric CD40L Fc fusion protein or fragment thereof with a vaccine antigen to obtain the vaccine composition.

In some embodiments, the single chain trimeric CD40L Fc fusion proteins or fragments thereof comprise IgG-like molecules with complementary CH3 domains that promote heterodimerization. In some embodiments, the single chain trimeric CD40L Fc fusion proteins or fragments thereof comprise recombinant IgG-like dual targeting molecules, wherein the two sides of the molecule each contain the Fab fragment or part of the Fab fragment of at least two different antibodies. In some embodiments, the single chain trimeric CD40L Fc fusion proteins or fragments thereof comprise IgG fusion molecules, wherein full length IgG antibodies are fused to an extra Fab fragment or parts of Fab fragment. In some embodiments, the single chain trimeric CD40L Fc fusion proteins or fragments thereof comprise Fc fusion molecules, wherein single chain Fv molecules or stabilized diabodies are fused to heavy-chain constant-domains, Fc-regions or parts thereof. In some embodiments, the single chain trimeric CD40L Fc fusion proteins or fragments thereof comprise Fab fusion molecules, wherein different Fab fragments are fused together. In some embodiments, the single chain trimeric CD40L Fc fusion proteins or fragments thereof comprise scFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus), the Azymetric™ platform (Zymeworks) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked mAbs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China).

“Homodimerization” as used herein refers to an interaction between the Fc monomer peptides of two identical single chain trimeric CD40L Fc fusion proteins. “Homodimer” as used herein refers to a molecule having identical single chain trimeric CD40L Fc fusion proteins.

“Heterodimerization” as used herein refers to an interaction between the Fc monomer peptides of single chain trimeric CD40L Fc fusion proteins with a non-identical Fc fusion peptide. “Heterodimer” as used herein refers to a single chain trimeric CD40L Fc fusion protein having two heavy chains with non-identical CH3 amino acid sequences.

The “knob-in-hole” strategy (see, e.g., PCT Publ. No. WO2006/028936) can be used to generate full length single chain trimeric CD40L Fc fusion proteins. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T3661_K392M_T394W/F405A_Y407V, T366L_K392M_T394W/F405A_Y407V, L351Y_Y407A/T366A_K409F, L351Y_Y407A/T366V_K409F_Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849.

In certain embodiments, the single chain trimeric CD40L Fc fusion proteins described herein activate CD40 with an EC₅₀ of less than about 1 pM, less than about 0.9 pM, less than about 0.8 pM, less than about 0.7 pM, less than about 0.6 pM, less than about 0.5 μM, less than about 0.4 pM, less than about 0.300 pM, less than about 0.2 pM, less than about 0.19 pM, less than about 0.18 pM, less than about 0.17 pM, less than about 0.16 pM, less than about 0.15 pM, less than about 0.14 pM, less than about 0.13 pM, less than about 0.12 pM, less than about 0.11 pM, less than about 0.1 pM, less than about 0.09 pM, less than about 0.08 pM, less than about 0.07 pM, less than about 0.06 pM, less than about 0.05 pM, less than about 0.04 pM, less than about 0.03 pM, less than about 0.02 pM, or less than about 0.01 pM. In certain embodiments, the EC₅₀ is less than about 1000 pM, less than about 900 pM, less than about 800 pM, less than about 700 pM, less than about 600 pM, less than about 500 pM, less than about 400 pM, less than about 300 pM, less than about 200 pM, less than about 190 μM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about 30 pM, less than about 20 pM, or less than about 10 pM.

In certain embodiments, the concentration of a single chain trimeric CD40L Fc fusion protein is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL.

In some embodiments, one or more components of a single chain trimeric CD40L Fc fusion protein is human. In some embodiments, one or more components of a single chain trimeric CD40L Fc fusion protein is humanized.

In some embodiments, the Fc monomer peptide of a single chain trimeric CD40L Fc fusion protein derived from an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In another general aspect, the invention relates to a vector comprising a polynucleotide encoding a single chain trimeric CD40L Fc fusion protein or fragment thereof. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments provided herein. Such techniques are well known to those skilled in the art in view of the present disclosure.

In another general aspect, the invention relates to a host cell comprising a vector comprising a polynucleotide encoding a single chain trimeric CD40L Fc fusion protein or fragment thereof. In another general aspect, the invention relates to a host cell comprising a polynucleotide encoding a single chain trimeric CD40L Fc fusion protein or fragment thereof. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an Fc monomer peptide), CHO-DG44 or CHO-K1 cells or HEK293 cells (for expression of, e.g., a single chain trimeric CD40L Fc fusion protein). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another aspect, the invention provides pharmaceutical compositions comprising the single chain trimeric CD40L Fc fusion proteins described herein, and a pharmaceutically acceptable carrier.

In another aspect, the invention provides pharmaceutical compositions comprising the means for delivering the single chain trimeric CD40L Fc fusion protein described herein, and a pharmaceutically acceptable carrier.

Also provided are methods of producing a pharmaceutical composition comprising combining the single chain trimeric CD40L Fc fusion proteins described herein with a pharmaceutically acceptable carrier to obtain the pharmaceutical composition. The term “pharmaceutical composition” as used herein means a product comprising a single chain trimeric CD40L Fc fusion protein provided herein together with a pharmaceutically acceptable carrier. Therefore, a pharmaceutical composition can comprise a single chain trimeric CD40L Fc fusion protein provided herein and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition provided herein. According to particular embodiments, in view of the present disclosure, any pharmaceutically acceptable carrier suitable for use in a pharmaceutical composition having a fusion protein as active ingredient can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment provided herein, the pH of the formulation is from about 7.0 to about 9.5. In another embodiment provided herein, the pH of the formulation is from about 3.0 to about 7.0.

In another embodiment provided herein, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments provided herein.

In another embodiment provided herein, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments provided herein.

In another embodiment provided herein, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative provided herein.

In another embodiment provided herein, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment provided herein, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment provided herein, the pharmaceutical composition comprises a stabilizer, wherein the stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments provided herein.

In further embodiments provided herein, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments provided herein.

In a further embodiment provided herein, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments provided herein.

In another general aspect, the invention relates to a method of producing a pharmaceutical composition comprising a single chain trimeric CD40L Fc fusion protein or fragment thereof disclosed herein, comprising combining comprising a single chain trimeric CD40L Fc fusion protein or fragment with a pharmaceutically acceptable carrier to obtain the pharmaceutical composition.

### 6. EMBODIMENTS

This invention provides the following non-limiting embodiments.

In one set of embodiments (embodiment set A), provided are:
A1. A single chain trimeric CD40L Fc fusion protein comprising (a) three CD40 ligand (CD40L) subunits covalently linked to one another by peptide linkers (CD40L trimer); and (b) an Fc monomer peptide.
A2. The single chain trimeric CD40L Fc fusion protein of embodiment A1, wherein the Fc monomer peptide is covalently linked to the CD40L trimer by a peptide tether.
A3. The single chain trimeric CD40L Fc fusion protein of embodiment A2, wherein the peptide tether comprises between 0 and 20 amino acids.
A4. The single chain trimeric CD40L Fc fusion protein of embodiment A1, wherein the CD40 ligand subunits comprise a portion of the CD40L extracellular domain.
A5. The single chain trimeric CD40L Fc fusion protein of embodiment A1, wherein the CD40L trimer is connected to the N-terminus of the Fc monomer peptide.
A6. The single chain trimeric CD40L Fc fusion protein of embodiment A5, wherein the single chain trimeric CD40L Fc fusion protein comprises any one sequence selected from SEQ ID NOS:1-12, or a fragment thereof.
A7. The single chain trimeric CD40L Fc fusion protein of embodiment A1, wherein the CD40L trimer is connected to the C-terminus of the Fc monomer peptide.
A8. The single chain trimeric CD40L Fc fusion protein of embodiment A7, wherein the single chain trimeric CD40L Fc fusion protein comprises any one sequence selected from SEQ ID NOS:13-19, or a fragment thereof.
A9. The single chain trimeric CD40L Fc fusion protein of embodiment A8, wherein the single chain trimeric CD40L Fc fusion protein comprises SEQ ID NO:16 or a fragment thereof.
A10. The single chain trimeric CD40L Fc fusion protein of embodiment A1, wherein the CD40 ligand subunits comprise any one of the sequences selected from SEQ ID NOS:20-22, or a fragment thereof.
A11. The single chain trimeric CD40L Fc fusion protein of embodiment A1, wherein the Fc monomer peptide comprises a human Fc sequence.
A12. The single chain trimeric CD40L Fc fusion protein of embodiment A11, wherein the human Fc sequence comprises a sequence selected from immunoglobulins IgG, IgA, IgM, IgD and IgE.
A13. The single chain trimeric CD40L Fc fusion protein of embodiment A12, wherein the human Fc sequence comprises an IgG sequence.
A14. The single chain trimeric CD40L Fc fusion protein of embodiment A13, wherein the IgG sequence is selected from IgG1, IgG2, IgG3 and IgG4.
A15. The single chain trimeric CD40L Fc fusion protein of embodiment A14, wherein the IgG sequence comprises an IgG1 sequence.
A16. The single chain trimeric CD40L Fc fusion protein of embodiment A15, wherein the IgG1 sequence comprises SEQ ID NOS:30 or 31, or a fragment thereof.
A17. The single chain trimeric CD40L Fc fusion protein of embodiment A14, wherein the IgG sequence comprises an IgG2 sequence.
A18. The single chain trimeric CD40L Fc fusion protein of embodiment A17, wherein the IgG2 sequence comprises SEQ ID NO:29 or a fragment thereof.
A19. The single chain trimeric CD40L Fc fusion protein of embodiment A1, wherein the peptide linker is selected from the group comprising of EGKSSGSGS (SEQ ID NO:23) and (G₃S)₃ (SEQ ID NO:25).
A20. The single chain trimeric CD40L Fc fusion protein of embodiment A2, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
A21. The single chain trimeric CD40L Fc fusion protein of any one of embodiments A1-A20, wherein the single chain trimeric CD40L Fc fusion protein enhances activation of a CD40 polypeptide compared to wild-type CD40L.
A22. The single chain trimeric CD40L Fc fusion protein of embodiment A21, wherein the activation of the CD40 polypeptide enhances the immune-stimulatory functions of dendritic cells, B cells, monocytes and macrophages.
A23. The single chain trimeric CD40L Fc fusion protein of embodiment A22, wherein the activation of the CD40 polypeptide comprises enhanced T cell activation compared to wild-type CD40L.
A24. The single chain trimeric CD40L Fc fusion protein of embodiment A23, wherein the activation of the CD40L polypeptide comprises enhanced dendritic cell activation compared to wild-type CD40L.
A25. The single chain trimeric CD40L Fc fusion protein of any of embodiments A1-A24, wherein the single chain trimeric CD40L Fc fusion protein enhances anti-tumor activity compared to wild-type CD40L.
A26. A dimer comprising two single chain trimeric CD40L Fc fusion proteins of any one of embodiments A1-A25.
A27. The dimer of embodiment A26, wherein the dimer is a homodimer.
A28. The dimer of embodiment A26, wherein the dimer is formed by association of the Fc monomer peptides.
A29. A polynucleotide encoding the single chain trimeric CD40L Fc fusion protein of any one of embodiments A1-A25.
A30. A vector comprising the polynucleotide of embodiment A29.
A31. A host cell comprising the vector of embodiment A30.
A32. A host cell comprising the polynucleotide of embodiment A29.
A33. A pharmaceutical composition comprising a pharmaceutically acceptable carrier and the single chain trimeric CD40L Fc fusion protein of any one of embodiments A1-A25.
A34. A kit comprising the single chain trimeric CD40L Fc fusion protein of any one of embodiments A1-A25.
A35. A single chain trimeric CD40L Fc fusion protein of any one of embodiments A1-A25, or the dimer of any one of embodiments 26 to 28, for use in therapy.
A36. A single chain trimeric CD40L Fc fusion protein of any one of embodiments A1-A25, or the dimer of any one of embodiments 26 to 28, for use in the treatment of a disease or disorder.
A37. A single chain trimeric CD40L Fc fusion protein of any one of embodiments A1-A25, or the dimer of any one of embodiments 26 to 28, for use in the elimination of a diseased cell in a subject.
A38. A single chain trimeric CD40L Fc fusion protein of embodiment A37, wherein the diseased cell is a cancer cell or a cell infected with a pathogen.
A39. A single chain trimeric CD40L Fc fusion protein of any one of embodiments A1-A25, or the dimer of any one of embodiments 26 to 28, for use in the treatment of cancer.
A40. A single chain trimeric CD40L Fc fusion protein of embodiment A39, wherein the cancer is a solid cancer or a liquid cancer.
A41. A single chain trimeric CD40L Fc fusion protein of embodiment A39, wherein the cancer is selected from the group consisting of melanoma, mesothelioma, advanced solid tumor and lymphoma.
A42. A single chain trimeric CD40L Fc fusion protein of embodiments A1-A25, or the dimer of any one of embodiments 26 to 28, for use in the treatment of an infection.
A43. A single chain trimeric CD40L Fc fusion protein of embodiment A42, wherein the infective pathogen is a virus, a bacterial, a fungus or a parasite.
A44. A single chain trimeric CD40L Fc fusion protein of embodiments A1-A25, or the dimer of any one of embodiments 26 to 28, for use in the administration of a vaccine composition.
A43. A single chain trimeric CD40L Fc fusion protein of embodiment A44, wherein the vaccine is a vaccine against a cancer, infective pathogen or an allergen.

In another set of embodiments (embodiment set B), provided are:
B1. A system comprising a means for providing a single chain trimeric CD40L Fc fusion protein comprising (a) three CD40L subunits covalently linked to one another by peptide linkers (CD40L trimer); and (b) an Fc monomer peptide.
B2. The system of embodiment B1, wherein the Fc monomer peptide is covalently linked to the CD40L trimer by a peptide tether.
B3. The system of embodiment B2, wherein the peptide tether comprises between 0 and 20 amino acids.
B4. The system of embodiment B1, wherein the CD40 ligand subunits comprise a portion of the CD40L extracellular domain.
B5. The system of embodiment B1, wherein the CD40L trimer is connected to the N-terminus of the Fc monomer peptide.
B6. The system of embodiment B5, wherein the single chain trimeric CD40L Fc fusion protein comprises any one sequence selected from SEQ ID NOS:1-12, or a fragment thereof.
B7. The system of embodiment B1, wherein the CD40L trimer is connected to the C-terminus of the Fc monomer peptide.
B8. The system of embodiment B7, wherein the single chain trimeric CD40L Fc fusion protein comprises any one sequence selected from SEQ ID NOS:13-19, or a fragment thereof.
B9. The system of embodiment B8, wherein the single chain trimeric CD40L Fc fusion protein comprises SEQ ID NO:16 or a fragment thereof.
B10. The system of embodiment B1, wherein the CD40 ligand subunits comprise any one of the sequences selected from SEQ ID NOS:20-22, or a fragment thereof.
B11. The system of embodiment B1, wherein the Fc monomer peptide comprises a human Fc sequence.
B12. The system of embodiment B11, wherein the human Fc sequence comprises a sequence selected from immunoglobulins IgG, IgA, IgM, IgD and IgE.
B13. The system of embodiment B12, wherein the human Fc sequence comprises an IgG sequence.
B14. The system of embodiment B13, wherein the IgG sequence is selected from IgG1, IgG2, IgG3 and IgG4.
B15. The system of embodiment B14, wherein the IgG sequence is an IgG1 sequence.
B16. The system of embodiment B15, wherein the IgG1 sequence comprises SEQ ID NOS:30 or 31, or a fragment thereof.
B17. The system of embodiment B14, wherein the IgG sequence comprises an IgG2 sequence.
B18. The system of embodiment B17, wherein the IgG2 sequence comprises SEQ ID NO:29 or a fragment thereof.
B19. The system of embodiment B1, wherein the peptide linker is selected from the group comprising of EGKSSGSGS (SEQ ID NO:23) and (G₃S)₃ (SEQ ID NO:25).
B20. The system of embodiment B2, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
B21. The system of any one of embodiments B1-B20, wherein the single chain trimeric CD40L Fc fusion protein enhances activation of a CD40 polypeptide compared to wild-type CD40L.
B22. The system of embodiment B21, wherein the activation of the CD40 polypeptide enhances the immune-stimulatory functions of dendritic cells, B cells, monocytes and macrophages.
B23. The system of embodiment B22, wherein the activation of the CD40 polypeptide comprises enhanced T cell activation compared to wild-type CD40L.
B24. The system of embodiment B22, wherein the activation of the CD40 polypeptide comprises enhanced dendritic cell activation compared to wild-type CD40L.
B25. The system of any one of embodiments B1-B24, wherein the single chain trimeric CD40L Fc fusion protein comprises enhanced anti-tumor activity compared to wild-type CD40L.
B26. A system comprising a means for providing a dimer comprising two single chain trimeric CD40L Fc fusion proteins, each comprising (a) three CD40L subunits covalently linked to one another by peptide linkers; and (b) an Fc monomer peptide.
B27. The system of embodiment B26, wherein the dimer is a homodimer.
B28. The system of embodiment B26, wherein the dimer is formed by association of the Fc monomer peptides.
B29. A system comprising a means for providing a polynucleotide encoding the single chain trimeric CD40L Fc fusion protein of any one of embodiments B1-B25.
B30. A system comprising a means for providing a vector comprising the polynucleotide of embodiment B29.
B31. A system comprising a means for providing a host cell comprising the vector of embodiment B30.
B32. A system comprising a means for providing a host cell comprising the polynucleotide of embodiment B29.
B33. A system comprising a means for providing a pharmaceutical composition comprising a pharmaceutically acceptable carrier and the single chain trimeric CD40L Fc fusion protein of any one of embodiments B1-B25.
B34. A system comprising a means for providing a kit comprising the single chain trimeric CD40L Fc fusion protein of any one of embodiments B1-B25.

In another set of embodiments (embodiment set C), provided are:
C1. A method of activating a CD40 polypeptide comprising contacting the CD40 polypeptide with a single chain trimeric CD40L Fc fusion protein comprising (a) three CD40L subunits covalently linked to one another by peptide linkers (CD40L trimer); and (b) an Fc monomer peptide, wherein said single chain trimeric CD40L Fc fusion protein activates the CD40 polypeptide upon binding.
C2. The method of embodiment C1, wherein the Fc monomer peptide is covalently linked to the CD40L trimer by a peptide tether.
C3. The method of embodiment C2, wherein the peptide tether comprises between 0 and 20 amino acids.
C4. The method of embodiment C1, wherein the CD40 ligand subunits comprise a portion of the CD40L extracellular domain.
C5. The method of embodiment C1, wherein the CD40L trimer is connected to the N-terminus of the Fc monomer peptide.
C6. The method of embodiment C5, wherein the single chain trimeric CD40L Fc fusion protein comprises any one sequence selected from SEQ ID NOS:1-12, or a fragment thereof.
C7. The method of embodiment C1, wherein the CD40L trimer is connected to the C-terminus of the Fc monomer peptide.
C8. The method of embodiment C7, wherein the single chain trimeric CD40L Fc fusion protein comprises any one sequence selected from SEQ ID NOS:13-19, or a fragment thereof.
C9. The method of embodiment C8, wherein the single chain trimeric CD40L Fc fusion protein comprises SEQ ID NO:16 or a fragment thereof.
C10. The method of embodiment C1, wherein the CD40 ligand subunits comprise any one of the sequences selected from SEQ ID NOS:20-22, or a fragment thereof.
C11. The method of embodiment C1, wherein the Fc monomer peptide comprises a human Fc sequence.
C12. The method of embodiment C11, wherein the human Fc sequence comprises a sequence selected from immunoglobulins IgG, IgA, IgM, IgD and IgE.
C13. The method of embodiment C12, wherein the human Fc sequence comprises an IgG sequence.
C14. The method of embodiment C13, wherein the IgG sequence is selected from IgG1, IgG2, IgG3 and IgG4.
C15. The method of embodiment C14, wherein the IgG sequence is an IgG1 sequence.
C16. The method of embodiment C15, wherein the IgG1 sequence comprises SEQ ID NOS:30 or 31, or a fragment thereof.
C17. The method of embodiment C14, wherein the IgG sequence comprises an IgG2 sequence.
C18. The method of embodiment C17, wherein the IgG2 sequence comprises SEQ ID NO:29 or a fragment thereof.
C19. The method of embodiment C1, wherein the peptide linker is selected from the group comprising of EGKSSGSGS (SEQ ID NO:23) and (G₃S)₃ (SEQ ID NO:25).
C20. The method of embodiment C2, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
C21. The method of any one of embodiments C1-C20, comprising enhanced activation of a CD40 polypeptide compared to wild-type CD40L.
C22. The method of embodiment C21, wherein the activation of the CD40 polypeptide enhances the immune-stimulatory functions of dendritic cells, B cells, monocytes and macrophages.
C23. The method of embodiment C21, wherein the activation of the CD40 polypeptide comprises enhanced T cell activation compared to wild-type CD40L.
C24. The method of embodiment C21, wherein the activation of the CD40 polypeptide comprises enhanced dendritic cell activation compared to wild-type CD40L.
C25. The method of any one of embodiments C1-C24, comprising enhanced anti-tumor activity compared to wild-type CD40L.
C26. A method of activating a CD40 polypeptide comprising contacting the CD40 polypeptide with a dimer comprising two single chain trimeric CD40L Fc fusion proteins, each comprising (a) three CD40L subunits covalently linked to one another by peptide linkers; and (b) an Fc monomer peptide, wherein said single chain trimeric CD40L Fc fusion protein dimer activates the CD40 polypeptide upon binding.
C27. The method of embodiment C26, further comprising administering a homodimer.
C28. The method of embodiment C26, wherein the dimer is formed by association of the Fc monomer peptides.
C29. The method of any one of embodiments C1-C28, wherein the method is performed in vitro.
C30. The method of any one of embodiments C1-C28, wherein said method is performed in vivo.
C31. The method of embodiment C30, further wherein said contacting comprises administering a pharmaceutical composition comprising a pharmaceutically acceptable carrier and the single chain trimeric CD40L Fc fusion proteins.
C32. The method of embodiment C31, wherein said contacting enhances an innate anti-neoplastic immune response.
C33. A method of activating a T-cell comprising contacting the T-cell with an antigen presenting cell in the presence of a single chain trimeric CD40L Fc fusion protein comprising (a) three CD40L subunits covalently linked to one another by peptide linkers (CD40L trimer); and (b) an Fc monomer peptide, wherein said antigen presenting cell expresses a CD40 polypeptide; and wherein said single chain trimeric CD40L Fc fusion protein activates the T cell upon binding the CD40 polypeptide.
C34. The method of embodiment C33, wherein the Fc monomer peptide is covalently linked to the CD40L trimer by a peptide tether.
C35. The method of embodiment C34, wherein the peptide tether comprises between 0 and 20 amino acids.
C36. The method of embodiment C33, wherein the CD40 ligand subunits comprise a portion of the CD40L extracellular domain.
C37. The method of embodiment C33, wherein the CD40L trimer is connected to the N-terminus of the Fc monomer peptide.
C38. The method of embodiment C37, wherein the single chain trimeric CD40L Fc fusion protein comprises any one sequence selected from SEQ ID NOS:1-12, or a fragment thereof.
C39. The method of embodiment C33, wherein the CD40L trimer is connected to the C-terminus of the Fc monomer peptide.
C40. The method of embodiment C39, wherein the single chain trimeric CD40L Fc fusion protein comprises any one sequence selected from SEQ ID NOS:13-19, or a fragment thereof.
C41. The method of embodiment C40, wherein the single chain trimeric CD40L Fc fusion protein comprises SEQ ID NO:16 or a fragment thereof.
C42. The method of embodiment C33, wherein the CD40 ligand subunits comprise any one of the sequences selected from SEQ ID NOS:20-22, or a fragment thereof.
C43. The method of embodiment C33, wherein the Fc monomer peptide comprises a human Fc sequence.
C44. The method of embodiment C43, wherein the human Fc sequence comprises a sequence selected from immunoglobulins IgG, IgA, IgM, IgD and IgE.
C45. The method of embodiment C44, wherein the human Fc sequence comprises an IgG sequence.
C46. The method of embodiment C45, wherein the IgG sequence is selected from IgG1, IgG2, IgG3 and IgG4.
C47. The method of embodiment C46, wherein the IgG sequence is an IgG1 sequence.
C48. The method of embodiment C47, wherein the IgG1 sequence comprises SEQ ID NOS:30 or 31, or a fragment thereof.
C49. The method of embodiment C46, wherein the IgG sequence comprises an IgG2 sequence.
C50. The method of embodiment C49, wherein the IgG2 sequence comprises SEQ ID NO:29 or a fragment thereof.
C51. The method of embodiment C33, wherein the peptide linker is selected from the group comprising of EGKSSGSGS (SEQ ID NO:23) and (G₃S)₃ (SEQ ID NO:25).
C52. The method of embodiment C34, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
C53. The method of any one of embodiments C33-C52, further comprising enhanced activation of the CD40 polypeptide compared to wild-type CD40L.
C54. The method of embodiment C53, wherein the enhanced activation of the CD40 polypeptide enhances the immune-stimulatory functions of dendritic cells, B cells, monocytes and macrophages.
C55. The method of embodiment C53, further comprising enhanced T cell activation compared to wild-type CD40L.
C56. The method of embodiment C53, further comprising enhanced dendritic cell activation compared to wild-type CD40L.
C57. The method of any one of embodiments C33-C56, wherein the single chain trimeric CD40L Fc fusion protein comprises enhanced anti-tumor activity compared to wild-type CD40L.
C58. A method of activating a T cell comprising contacting the CD40 polypeptide with an antigen presenting cell in the presence of a dimer comprising two single chain trimeric CD40L Fc fusion proteins, each comprising (a) three CD40L subunits covalently linked to one another by peptide linkers; and (b) an Fc monomer peptide, wherein said antigen presenting cell expresses a CD40 polypeptide, and wherein said single chain trimeric CD40L Fc fusion protein dimer activates the T cell upon binding the CD40 polypeptide.
C59. The method of embodiment C58 further comprising administering a homodimer.
C60. The method of embodiment C58, wherein the dimer is formed by association of the Fc monomer peptides.
C61. The method of any one of embodiments C33-C60, wherein the method is performed in vitro.
C62. The method of any one of embodiments C33-C60, wherein said method is performed in vivo.
C63. The method of any one of embodiments C62, further wherein said contacting comprises administering a pharmaceutical composition comprising a pharmaceutically acceptable carrier and the single chain trimeric CD40L Fc fusion proteins.
C64. The method of embodiment C63, wherein said contacting enhances an innate anti-neoplastic immune response.
C65. A method of activating a dendritic cell comprising contacting the CD40 polypeptide with a single chain trimeric CD40L Fc fusion protein comprising (a) three CD40L subunits covalently linked to one another by peptide linkers (CD40L trimer); and (b) an Fc monomer peptide, wherein said single chain trimeric CD40L Fc fusion protein activates the dendritic cell upon binding the CD40 polypeptide.
C66. The method of embodiment C65, wherein the Fc monomer peptide is covalently linked to the CD40L trimer by a peptide tether.
C67. The method of embodiment C66, wherein the peptide tether comprises between 0 and 20 amino acids.
C68. The method of embodiment C65, wherein the CD40 ligand subunits comprise a portion of the CD40L extracellular domain.
C69. The method of embodiment C65, wherein the CD40L trimer is connected to the N-terminus of the Fc monomer peptide.
C70. The method of embodiment C69, wherein the single chain trimeric CD40L Fc fusion protein comprises any one sequence selected from SEQ ID NOS:1-12, or a fragment thereof.
C71. The method of embodiment C65, wherein the CD40L trimer is connected to the C-terminus of the Fc monomer peptide.
C72. The method of embodiment C71, wherein the single chain trimeric CD40L Fc fusion protein comprises any one sequence selected from SEQ ID NOS:13-19, or a fragment thereof.
C73. The method of embodiment C72, wherein the single chain trimeric CD40L Fc fusion protein comprises SEQ ID NO:16 or a fragment thereof.
C74. The method of embodiment C65, wherein the CD40 ligand subunits comprise any one of the sequences selected from SEQ ID NOS:20-22, or a fragment thereof.
C75. The method of embodiment C65, wherein the Fc monomer peptide comprises a human Fc sequence.
C76. The method of embodiment C75, wherein the human Fc sequence comprises a sequence selected from immunoglobulins IgG, IgA, IgM, IgD and IgE.
C77. The method of embodiment C76, wherein the human Fc sequence comprises an IgG sequence.
C78. The method of embodiment C77, wherein the IgG sequence is selected from IgG1, IgG2, IgG3 and IgG4.
C79. The method of embodiment C78, wherein the IgG sequence is an IgG1 sequence.
C80. The method of embodiment C79, wherein the IgG1 sequence comprises SEQ ID NOS:30 or 31, or a fragment thereof.
C81. The method of embodiment C78, wherein the IgG sequence comprises an IgG2 sequence.
C82. The method of embodiment C81, wherein the IgG2 sequence comprises SEQ ID NO:29 or a fragment thereof.
C83. The method of embodiment C65, wherein the peptide linker is selected from the group comprising of EGKSSGSGS (SEQ ID NO:23) and (G₃S)₃ (SEQ ID NO:25).
C84. The method of embodiment C66, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
C85. The method of any one of embodiments C65-C84, comprising enhanced activation of a CD40 polypeptide compared to wild-type CD40L.
C86. The method of embodiment C85, wherein the enhanced activation of the CD40 polypeptide enhances the immune-stimulatory functions of dendritic cells, B cells, monocytes and macrophages.
C87. The method of embodiment C85, further comprising enhanced T cell activation compared to wild-type CD40L.
C88. The method of embodiment C85, further comprising enhanced dendritic cell activation compared to wild-type CD40L.
C89. The method of any one of embodiments C65-C88, wherein the single chain trimeric CD40L Fc fusion protein comprises enhanced anti-tumor activity compared to wild-type CD40L.
C90. A method of activating a dendritic cell comprising contacting the CD40 polypeptide with a dimer comprising two single chain trimeric CD40L Fc fusion proteins, each comprising (a) three CD40L subunits covalently linked to one another by peptide linkers; and (b) an Fc monomer peptide, wherein said single chain trimeric CD40L Fc fusion protein dimer activates the dendritic cell upon binding the CD40 polypeptide.
C91. The method of embodiment C90 further comprising administering a homodimer.
C92. The method of embodiment C90, wherein the dimer is formed by association of the Fc monomer peptides.
C93. The method of any one of embodiments C54-C92, wherein the method is performed in vitro.
C94. The method of any one of embodiments C54-C92, wherein said method is performed in vivo.
C95. The method of any one of embodiments C94, further wherein said contacting comprises administering a pharmaceutical composition comprising a pharmaceutically acceptable carrier and the single chain trimeric CD40L Fc fusion proteins.
C96. The method of embodiment C95, wherein said contacting enhances an innate anti-neoplastic immune response.

In another set of embodiments (embodiment set D), provided are:
D1. A method of treating cancer in a subject comprising administering to the subject a therapeutically effective amount of a single chain trimeric CD40L Fc fusion protein comprising (a) three CD40L subunits covalently linked to one another by peptide linkers (CD40L trimer); and (b) an Fc monomer peptide.
D2. The method of embodiment D1, wherein the Fc monomer peptide is covalently linked to the CD40L trimer by a peptide tether.
D3. The method of embodiment D2, wherein the peptide tether comprises between 0 and 20 amino acids.
D4. The method of embodiment D1, wherein the CD40 ligand subunits comprise a portion of the CD40L extracellular domain.
D5. The method of embodiment D1, wherein the CD40L trimer is connected to the N-terminus of the Fc monomer peptide.
D6. The method of embodiment D5, wherein the single chain trimeric CD40L Fc fusion protein comprises any one sequence selected from SEQ ID NOS:1-12, or a fragment thereof.
D7. The method of embodiment D1, wherein the CD40L trimer is connected to the C-terminus of the Fc monomer peptide.
D8. The method of embodiment D7, wherein the single chain trimeric CD40L Fc fusion protein comprises any one sequence selected from SEQ ID NOS:13-19, or a fragment thereof.
D9. The method of embodiment D8, wherein the single chain trimeric CD40L Fc fusion protein comprises SEQ ID NO:16 or a fragment thereof.
D10. The method of embodiment D1, wherein the CD40 ligand subunits comprise any one of the sequences selected from SEQ ID NOS:20-22, or a fragment thereof.
D11. The method of embodiment D1, wherein the Fc monomer peptide comprises a human Fc sequence.
D12. The method of embodiment D11, wherein the human Fc sequence comprises a sequence selected from immunoglobulins IgG, IgA, IgM, IgD and IgE.
D13. The method of embodiment D12, wherein the human Fc sequence comprises an IgG sequence.
D14. The method of embodiment D13, wherein the IgG sequence is selected from IgG1, IgG2, IgG3 and IgG4.
D15. The method of embodiment D14, wherein the IgG sequence is an IgG1 sequence.
D16. The method of embodiment D15, wherein the IgG1 sequence comprises SEQ ID NOS:30 or 31, or a fragment thereof.
D17. The method of embodiment D14, wherein the IgG sequence comprises an IgG2 sequence.
D18. The method of embodiment D17, wherein the IgG2 sequence comprises SEQ ID NO:29 or a fragment thereof.
D19. The method of embodiment D1, wherein the peptide linker is selected from the group comprising of EGKSSGSGS (SEQ ID NO:23) and (G₃S)₃ (SEQ ID NO:25).
D20. The method of embodiment D2, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
D21. The method of any one of embodiments D1-D20 wherein the single chain trimeric CD40L Fc fusion protein comprises enhanced activation of a CD40 polypeptide compared to wild-type CD40L.
D22. The method of embodiment D21, wherein the enhanced activation of the CD40 polypeptide enhances the immune-stimulatory functions of dendritic cells, B cells, monocytes and macrophages.
D23. The method of embodiment D22, wherein the enhanced activation of the CD40 polypeptide comprises enhanced T cell activation compared to wild-type CD40L.
D24. The method of embodiment D22, wherein the single chain trimeric CD40L Fc fusion protein comprises enhanced dendritic cell activation compared to wild-type CD40L.
D25. The method of any one of embodiments D1-D24, wherein the single chain trimeric CD40L Fc fusion protein comprises enhanced anti-tumor activity compared to wild-type CD40L.
D26. A method of treating cancer in a subject comprising administering to the subject a therapeutically effective amount of a dimer comprising two single chain trimeric CD40L Fc fusion proteins, each comprising (a) three CD40L subunits covalently linked to one another by peptide linkers; and (b) an Fc monomer peptide.
D27. The method of embodiment D26 further comprising administering a homodimer.
D28. The method of embodiment D26, wherein the dimer is formed by association of the Fc monomer peptides.
D29. The method of any one of embodiments D1-D28, further comprising administering a pharmaceutical composition comprising a pharmaceutically acceptable carrier and the single chain trimeric CD40L Fc fusion proteins.
D30. The method of any one of embodiments D1-D29, wherein the treatment enhances an innate anti-neoplastic immune response.
D31. The method of any one of embodiments D1-D30, further comprising co-administration of a second therapy.
D32. The method of any one of embodiments D1-D31, wherein said cancer is selected from the group consisting of melanoma, mesothelioma, advanced solid tumor and lymphoma.

In another set of embodiments (embodiment set E), provided are:
E1. A method for producing a single chain trimeric CD40L Fc fusion protein or fragment thereof comprising (a) introducing into a host cell a polynucleotide encoding a single chain trimeric CD40L Fc fusion protein comprising (i) three CD40L subunits covalently linked to one another by peptide linkers (CD40L trimer); and (ii) an Fc monomer peptide; (b) culturing the host cell under conditions to produce the single chain trimeric CD40L Fc fusion protein or fragment thereof, and (c) recovering the single chain trimeric CD40L Fc fusion protein or fragment thereof from the cell or culture.
E2. The method of embodiment E1, wherein the Fc monomer peptide is covalently linked to the CD40L trimer by a peptide tether.
E3. The method of embodiment E2, wherein the peptide tether comprises between 0 and 20 amino acids.
E4. The method of embodiment E1, wherein the CD40 ligand subunits comprise a portion of the CD40L extracellular domain.
E5. The method of embodiment E1, wherein the CD40L trimer is connected to the N-terminus of the Fc monomer peptide.
E6. The method of embodiment E5, wherein the single chain trimeric CD40L Fc fusion protein comprises any one sequence selected from SEQ ID NOS:1-12, or a fragment thereof.
E7. The method of embodiment E1, wherein the CD40L trimer is connected to the C-terminus of the Fc monomer peptide.
E8. The method of embodiment E7, wherein the single chain trimeric CD40L Fc fusion protein comprises any one sequence selected from SEQ ID NOS:13-19, or a fragment thereof.
E9. The method of embodiment E8, wherein the single chain trimeric CD40L Fc fusion protein comprises SEQ ID NO:16 or a fragment thereof.
E10. The method of embodiment E1, wherein the CD40 ligand subunits comprise any one of the sequences selected from SEQ ID NOS:20-22, or a fragment thereof.
E11. The method of embodiment E1, wherein the Fc monomer peptide comprises a human Fc sequence.
E12. The method of embodiment E11, wherein the human Fc sequence comprises a sequence selected from immunoglobulins IgG, IgA, IgM, IgD and IgE.
E13. The method of embodiment E12, wherein the human Fc sequence comprises an IgG sequence.
E14. The method of embodiment E13, wherein the IgG sequence is selected from IgG1, IgG2, IgG3 and IgG4.
E15. The method of embodiment E14, wherein the IgG sequence is an IgG1 sequence.
E16. The method of embodiment E15, wherein the IgG1 sequence comprises SEQ ID NOS:30 or 31, or a fragment thereof.
E17. The method of embodiment E14, wherein the IgG sequence comprises an IgG2 sequence.
E18. The method of embodiment E17, wherein the IgG2 sequence comprises SEQ ID NO:29 or a fragment thereof.
E19. The method of embodiment E1, wherein the peptide linker is selected from the group comprising of EGKSSGSGS (SEQ ID NO:23) and (G₃S)₃ (SEQ ID NO:25).
E20. The method of embodiment E2, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
E21. The method of any one of embodiments E1-E20, wherein the single chain trimeric CD40L Fc fusion protein enhances activation of a CD40 polypeptide compared to wild-type CD40L.
E22. The method of embodiment E21, wherein the activation of the CD40 polypeptide enhances the immune-stimulatory functions of dendritic cells, B cells, monocytes and macrophages.
E23. The method of embodiment E22, wherein the activation of the CD40 polypeptide comprises enhanced T cell activation compared to wild-type CD40L.
E24. The method of embodiment E23, wherein the activation of the CD40 polypeptide comprises enhanced dendritic cell activation compared to wild-type CD40L.
E25. The method of embodiment E1, wherein the single chain trimeric CD40L Fc fusion protein comprises enhanced anti-tumor activity compared to wild-type CD40L.
E26. A method of producing a dimer comprising two single chain trimeric CD40L Fc fusion proteins, each comprising (a) introducing into a host cell a polynucleotide encoding a single chain trimeric CD40L Fc fusion protein comprising (i) three CD40L subunits covalently linked to one another by peptide linkers; and (ii) an Fc monomer peptide; (b) culturing the host cell under conditions to produce the single chain trimeric CD40L Fc fusion protein or fragment thereof; (c) recovering the single chain trimeric CD40L Fc fusion protein or fragment thereof from the cell or culture, and (d) combining single chain trimeric CD40L Fc fusion proteins or fragments thereof under conditions that favor dimerization.
E27. The method of embodiment E26, wherein the dimer comprises a homodimer.
E28. The method of embodiment E26, wherein the dimer is formed by association of the Fc monomer peptides.
E29. A method of producing a pharmaceutical composition of a single chain trimeric CD40L Fc fusion protein or fragment thereof comprising combining the single chain trimeric CD40L Fc fusion protein produced according to any one of embodiments E1 to E25 or fragment thereof with a pharmaceutically acceptable carrier to obtain the pharmaceutical composition.

In another set of embodiments (embodiment set F), provided are:
F1. A method of increasing an immune response in a subject comprising administering to the subject a therapeutically effective amount of a single chain trimeric CD40L Fc fusion protein comprising (a) three CD40L subunits covalently linked to one another by peptide linkers; and (b) an Fc monomer peptide.
F2. A method of increasing an immune response to a cancer antigen in a subject having cancer comprising administering to the subject a therapeutically effective amount of a single chain trimeric CD40L Fc fusion protein comprising (a) three CD40L subunits covalently linked to one another by peptide linkers; and (b) an Fc monomer peptide.
F3. A method of increasing an immune response to a pathogen in a subject infected with the pathogen comprising administering to the subject a therapeutically effective amount of a single chain trimeric CD40L Fc fusion protein comprising (a) three CD40L subunits covalently linked to one another by peptide linkers; and (b) an Fc monomer peptide.
F4. A method of increasing an immune response to a vaccine antigen in a subject being administered a vaccine, wherein the method further comprises administering to the subject a therapeutically effective amount of a single chain trimeric CD40L Fc fusion protein comprising (a) three CD40L subunits covalently linked to one another by peptide linkers; and (b) an Fc monomer peptide.
F5. The method of any one of embodiments F1 to F4, wherein the peptide tether comprises between 0 and 20 amino acids.
F6. The method of any one of embodiments F1 to F5, wherein the CD40 ligand subunits comprise a portion of the CD40L extracellular domain.
F7. The method of any one of embodiments F1 to F6, wherein the CD40L trimer is connected to the N-terminus of the Fc monomer peptide.
F8. The method of any one of embodiments F1 to F7, wherein the single chain trimeric CD40L Fc fusion protein comprises any one sequence selected from SEQ ID NOS:1-12, or a fragment thereof.
F9. The method of any one of embodiments F1 to F8, wherein the CD40L trimer is connected to the C-terminus of the Fc monomer peptide.
F10. The method of any one of embodiments F1 to F9, wherein the single chain trimeric CD40L Fc fusion protein comprises any one sequence selected from SEQ ID NOS:13-19, or a fragment thereof.
F11. The method of any one of embodiments F1 to F10, wherein the single chain trimeric CD40L Fc fusion protein comprises SEQ ID NO:16 or a fragment thereof.
F12. The method of any one of embodiments F1 to F11, wherein the CD40 ligand subunits comprise any one of the sequences selected from SEQ ID NOS:20-22, or a fragment thereof.
F13. The method of any one of embodiments F1 to F12, wherein the Fc monomer peptide comprises a human Fc sequence.
F14. The method of embodiment F13, wherein the human Fc sequence comprises a sequence selected from immunoglobulins IgG, IgA, IgM, IgD and IgE.
F15. The method of embodiment F14, wherein the human Fc sequence comprises an IgG sequence.
F16. The method of embodiment F15, wherein the IgG sequence is selected from IgG1, IgG2, IgG3 and IgG4.
F17. The method of embodiment F16, wherein the IgG sequence is an IgG1 sequence.
F18. The method of embodiment F17, wherein the IgG1 sequence comprises SEQ ID NOS:30 or 31, or a fragment thereof.
F19. The method of embodiment F16, wherein the IgG sequence comprises an IgG2 sequence.
F20. The method of embodiment F19, wherein the IgG2 sequence comprises SEQ ID NO:29 or a fragment thereof.
F21. The method of any one of embodiments F1 to F20, wherein the peptide linker is selected from the group comprising of EGKSSGSGS (SEQ ID NO:23) and (G₃S)₃ (SEQ ID NO:25).
F22. The method of any one of embodiments F1 to F20, wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).
F23. The method of any one of embodiments F1-F22 wherein the single chain trimeric CD40L Fc fusion protein comprises enhanced activation of a CD40 polypeptide compared to wild-type CD40L.
F24. The method of embodiment F23, wherein the enhanced activation of the CD40 polypeptide enhances the immune-stimulatory functions of dendritic cells, B cells, monocytes and macrophages.
F25. The method of embodiment F24, wherein the enhanced activation of the CD40 polypeptide comprises enhanced T cell activation compared to wild-type CD40L.
F26. The method of embodiment F22, wherein the single chain trimeric CD40L Fc fusion protein comprises enhanced dendritic cell activation compared to wild-type CD40L.
F27. The method of any one of embodiments F1-F26, wherein the single chain trimeric CD40L Fc fusion protein comprises enhanced anti-tumor activity compared to wild-type CD40L.
F28. A method of increasing an immune response in a subject comprising administering to the subject a therapeutically effective amount of a dimer comprising two single chain trimeric CD40L Fc fusion proteins, each comprising (a) three CD40L subunits covalently linked to one another by peptide linkers; and (b) an Fc monomer peptide.
F29. A method of increasing an immune response to a cancer antigen in a subject having cancer comprising administering to the subject a therapeutically effective amount of a dimer comprising two single chain trimeric CD40L Fc fusion proteins, each comprising (a) three CD40L subunits covalently linked to one another by peptide linkers; and (b) an Fc monomer peptide.
F30. A method of increasing an immune response to a pathogen in a subject infected with the pathogen comprising administering to the subject a therapeutically effective amount of a dimer comprising two single chain trimeric CD40L Fc fusion proteins, each comprising (a) three CD40L subunits covalently linked to one another by peptide linkers; and (b) an Fc monomer peptide.
F31. A method of increasing an immune response to a vaccine antigen in a subject being administered a vaccine, wherein the method further comprises administering to the subject a therapeutically effective amount of a dimer comprising two single chain trimeric CD40L Fc fusion proteins, each comprising (a) three CD40L subunits covalently linked to one another by peptide linkers; and (b) an Fc monomer peptide.
F32. The method of any one of embodiments F28 to F31, further comprising administering a homodimer.
F33. The method of any one of embodiments F28 to F32, wherein the dimer is formed by association of the Fc monomer peptides.
F34. The method of any one of embodiments F28 to F33, further comprising administering a pharmaceutical composition comprising a pharmaceutically acceptable carrier and the single chain trimeric CD40L Fc fusion proteins.
F35. The method of any one of embodiments F28 to F34, further comprising co-administration of a second therapy.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### 7. EXAMPLES

### Example 1: Methods and Materials

Design of soluble trimeric CD40L Fc fusion constructs: CD40L is a membrane protein consisting of an extracellular domain, stalk region, transmembrane helix, and short cytoplasmic domain. The activity of CD40L is located to the extracellular domain, and this domain was chosen for Fc fusion construct generation. CD40L ECDs (Uniprot ID: P29965) were fused together with flexible linkers and tethers to form N-terminal fusions with effector function silenced human IgG1 and human IgG2 isotypes. The linker between the CD40L subunits was either 9 or 12 amino acids and was chosen for flexibility, though no other lengths were tested. The tether between the last CD40L subunit and the Fc was chosen to promote flexibility and ranged from 0-20 amino acids. Additionally, C-terminal Fc fusions of CD40L were generated. Flexible tethers ranging from 5-20 amino acids were placed between the C-terminus of the Fc and the N-terminus of the first CD40L subunit. Flexible linkers of either 9 or 12 amino acids were used between the CD40L subunits. The C-terminal trimeric CD40L Fc fusions were constructed on effector silenced human IgG1 and IgG2A isotypes. Initial attempts to design an agonist CD40L with CD40L trimer bundle fused to huIgG molecule resulted in high molecular weight species (HMWS) after protein A purification and were unsuitable for further development. The biophysical and functional properties of the protein were optimized by redesigning the protein with structure based rationale. The proteins were designed with various intra-subunit linkers, tethers and Fc attachment points (FIG. 1A).

Furthermore, to test the hypothesis that a CD40L trimer fusion protein had activity by itself, (i.e., without forming a dimer of two copies of the CD40L trimer fusion proteins through the interaction between their Fc portions), molecules that contained a CD40L trimer fused to a Fc monomer peptide with mutations in the CH3 domain that abrogated dimer formation were also designed and constructed (“Mono Fc” molecules, see e.g., FIG. 1B).

### Example 2: Expression and Purification

Expi293F cells were grown in serum-free Expi293™ Expression Medium (Invitrogen). The cells were maintained in Erlenmeyer Flasks at 37° C. with 8% C02 on an orbital shaker. Cells were transfected at 3.0×10⁶ cells/ml. Plasmid DNA was diluted in Opti-MEM I. Expifectamine 293 was diluted in Opti-MEM I and incubated at room temp for 5 min. The DNA mixture was incubated with Expifectamine 293 and incubated at room temperature for 20-30 min. The DNA/Expifectamine 293 complexation was added to cell culture and returned to the incubator. 16-18 h post-transfection appropriate volume of Enhancer was added to cell cultures. The cell culture supernatant was harvested on day 6 harvested by centrifuging at 3000 g for 10 mins to pellet cells. The supernatants were stored at 4° C. until purification.

RoboColumn Eshmuno A, 0.6 ml (MERCK MILLIPORE, Cat. No. 1.25163.0001) columns with Tecan liquid handler were used to purify protein. Supernatants were applied to the column at a flow rate of 0.6 mL/min for maximum capture. Columns were washed using 8 column volumes of PBS until a clean baseline was obtained as monitored by UV A280. Proteins were eluted off the column using 50 mM Citrate (pH3.0) and neutralized with 1M Tris-HCl (pH 9.0). The proteins were desalted using Zebra Spin Desalting columns into dPBS as final buffer. Fractions were tested for the presence of recombinant protein using non-denaturing and denaturing SDS PAGE gels and pooled. Proteins purified were deemed >80% pure by SDS PAGE and analytical size exchange column (aSEC). Second step chromatographic polishing was accomplished by Superdex200 gel filtration chromatography using PBS mobile phase. Fractions corresponding to ˜150 kDa were pooled and stored at −80° C. until use.

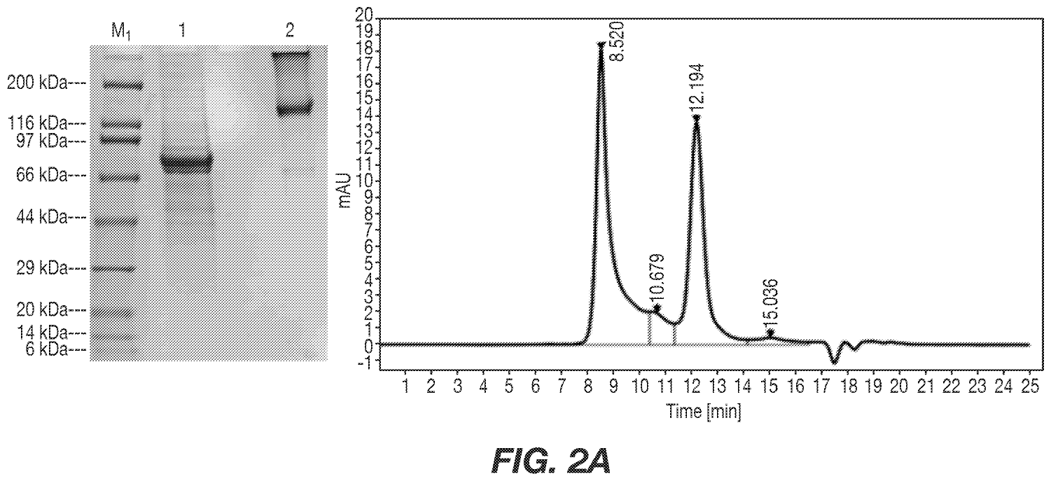

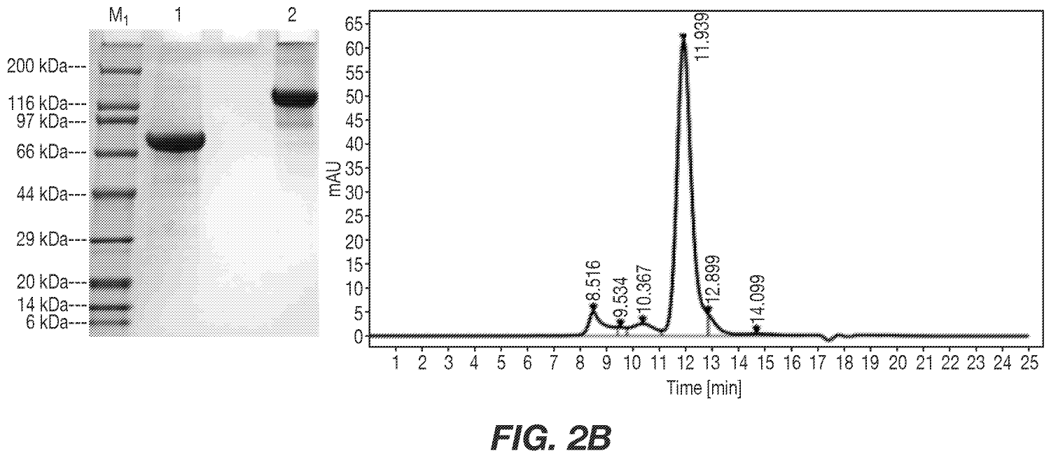

The newly designed trimers showed significant improvement in purity as determined by the level of monodisperse Fc homodimer using HPLC (aSEC) (Table 1, below), and SDS-PAGE gel (FIGS. 2A-2B).

TABLE 1
Monodispersity of Engineered CD40L Trimer Fused to
Fc as Measured by aSEC Post Protein A Purification

| Protein AA | % Monomer post Protein A |
|---|---|
| TPP000182980 | 79.36 |
| TPP000182981 | 79.01 |
| TPP000182982 | 73.61 |
| TPP000182983 | 81.2 |
| TPP000182984 | 77.2 |
| TPP000182985 | 77.68 |
| TPP000182986 | 80.23 |
| TPP000182988 | 70.59 |
| TPP000182989 | 76.75 |
| TPP000182990 | 78.66 |
| TPP000182991 | 77.21 |
| TPP000182992 | 76.2 |
| TPP000182993 | 65.74 |
| TPP000182994 | 75.82 |
| TPP000182995 | 78.92 |
| TPP000182996 | 76.59 |
| TPP000182997 | 71.78 |
| TPP000182021 | 79.12 |
| TPP000161222 | 38.76 |

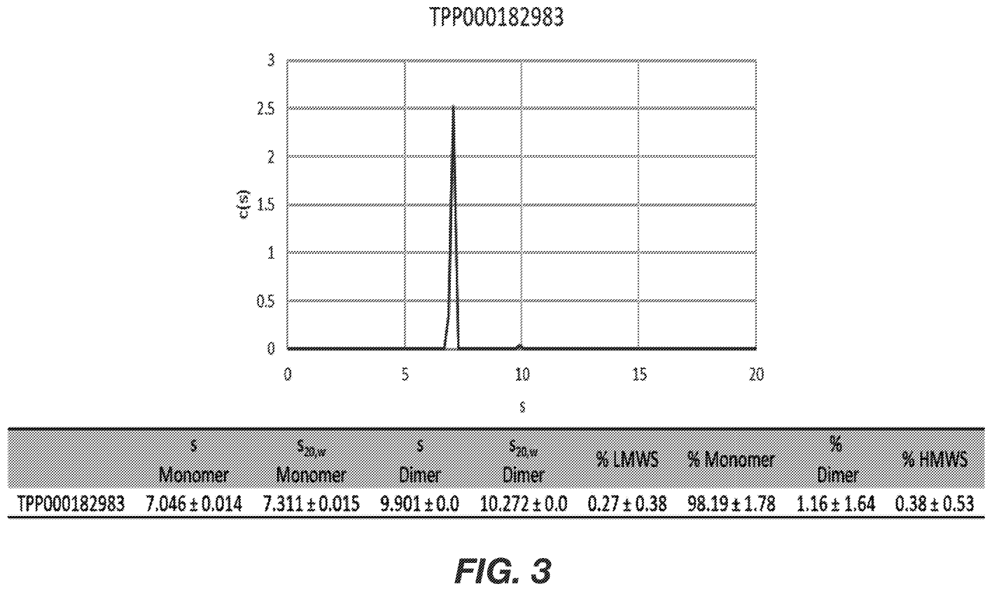

The original molecule possessed 38% purity post protein A purification (TPP000161222) determined by aSEC and showed a prominent high molecular species band in SDS-PAGE gel (FIGS. 2A-2B). In contrast, the newly designed trimers showed a range of purity, from 65-81% as determined by aSEC (Table 1). The trimers were further polished using size exclusion chromatography where TPP000161222 showed 9800 monodispersity when measured by analytical ultra-centrifugation (AUC) with less than 20% undesired species including high and low molecular weight species (FIG. 3).

Sequences of Constructs:

Font Scheme: CD40L-LINKER-CD40L-LINKER-CD40L-TETHER-HUMAN IgG Fc
TPP000183311|OVTSB10|heavy_chain|hsctCD40LG2_EKWv2NPQ
(SEQ ID NO: 1)
NPQIAAHVISEASSKTTSVLQWAEKGYYTMSNNLVTLENGKQLTVKRQGLYYIYAQ
VTFCSNREASSQAPFIASLWLKSPGRFERILLRAANTHSSAKPCGQQSIHLGGVFELQP
GASVFVNVTDPSQVSHGTGFTSFGLLKLEGKSSGSGSQIAAHVISEASSKTTSVLQWA
EKGYYTMSNNLVTLENGKQLTVKRQGLYYIYAQVTFCSNREASSQAPFIASLWLKSP
GRFERILLRAANTHSSAKPCGQQSIHLGGVFELQPGASVFVNVTDPSQVSHGTGFTSF
GLLKLEGKSSGSGSQIAAHVISEASSKTTSVLQWAEKGYYTMSNNLVTLENGKQLTV
KRQGLYYIYAQVTFCSNREASSQAPFIASLWLKSPGRFERILLRAANTHSSAKPCGQQ
SIHLGGVFELQPGASVFVNVTDPSQVSHGTGFTSFGLLKLVERKSCVECPPCPAPPV
AGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK
>TPP000183310|OVTSB9|heavy_chain|hsctCD40LG2_EKWv2NPQ_G4S3
(SEQ ID NO: 2)
NPQIAAHVISEASSKTTSVLQWAEKGYYTMSNNLVTLENGKQLTVKRQGLYYIYAQ
VTFCSNREASSQAPFIASLWLKSPGRFERILLRAANTHSSAKPCGQQSIHLGGVFELQP
GASVFVNVTDPSQVSHGTGFTSFGLLKLEGKSSGSGSQIAAHVISEASSKTTSVLQWA
EKGYYTMSNNLVTLENGKQLTVKRQGLYYIYAQVTFCSNREASSQAPFIASLWLKSP
GRFERILLRAANTHSSAKPCGQQSIHLGGVFELQPGASVFVNVTDPSQVSHGTGFTSF
GLLKLEGKSSGSGSQIAAHVISEASSKTTSVLQWAEKGYYTMSNNLVTLENGKQLTV
KRQGLYYIYAQVTFCSNREASSQAPFIASLWLKSPGRFERILLRAANTHSSAKPCGQQ
SIHLGGVFELQPGASVFVNVTDPSQVSHGTGFTSFGLLKLGGGGSGGGGSGGGGSV
ERKSCVECPPCPAPPVAGPSVFLFPPKPKDTLMISRTPEVTCVVVDVSHEDPEVQ
FNWYVDGVEVHNAKTKPREEQFNSTFRVVSVLTVVHQDWLNGKEYKCKVSNK
GLPAPIEKTISKTKGQPREPQVYTLPPSREEMTKNQVSLTCLVKGFYPSDIAVEW
ESNGQPENNYKTTPPMLDSDGSFFLYSKLTVDKSRWQQGNVFSCSVMHEALHN
HYTQKSLSLSPGK
>TPP000182997|C4LW30|mature_protein|NPQ_G3S3_sthCD40L_G4S2_IgG1_AAS
(SEQ ID NO: 3)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLGGGSGGGSGGGSNPQIAAHVISEASSKTTS
VLQWAEKGYYTMSNNLVTLENGKQLTVKRQGLYYIYAQVTFCSNREASSQAPFIAS
LCLKSPGRFERILLRAANTHSSAKPCGQQSIHLGGVFELQPGASVFVNVTDPSQVSHG
TGFTSFGLLKLGGGSGGGSGGGSNPQIAAHVISEASSKTTSVLQWAEKGYYTMSNNL
VTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKLGGGGSG
GGGSEPKSSDKTHTCPPCPAPEAAGGPSVFLFPPKPKDTLMISRTPEVTCVVVSV
SHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGK
>TPP000182996|C4LW29|mature_protein|NPQ_G3S3_sthCD40L_G4S3_IgG1_AAS
(SEQ ID NO: 4)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLGGGSGGGSGGGSNPQIAAHVISEASSKTTS
VLQWAEKGYYTMSNNLVTLENGKQLTVKRQGLYYIYAQVTFCSNREASSQAPFIAS
LCLKSPGRFERILLRAANTHSSAKPCGQQSIHLGGVFELQPGASVFVNVTDPSQVSHG
TGFTSFGLLKLGGGSGGGSGGGSNPQIAAHVISEASSKTTSVLQWAEKGYYTMSNNL
VTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKLGGGGSG
GGGSGGGGSEPKSSDKTHTCPPCPAPEAAGGPSVFLFPPKPKDTLMISRTPEVTC
VVVSVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGN
VFSCSVMHEALHNHYTQKSLSLSPGK
>TPP000182995|C4LW28|mature_protein|NPQ_G3S3_sthCD40L_G4S4_IgG1_AAS
(SEQ ID NO: 5)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLGGGSGGGSGGGSNPQIAAHVISEASSKTTS
VLQWAEKGYYTMSNNLVTLENGKQLTVKRQGLYYIYAQVTFCSNREASSQAPFIAS
LCLKSPGRFERILLRAANTHSSAKPCGQQSIHLGGVFELQPGASVFVNVTDPSQVSHG
TGFTSFGLLKLGGGSGGGSGGGSNPQIAAHVISEASSKTTSVLQWAEKGYYTMSNNL
VTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKLGGGGSG
GGGSGGGGSGGGGSEPKSSDKTHTCPPCPAPEAAGGPSVFLFPPKPKDTLMISR
TPEVTCVVVSVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSR
WQQGNVFSCSVMHEALHNHYTQKSLSLSPGK
>TPP000182994|C4LW27|mature_protein|NPQ_G3S3_sthCD40L_G4S_IgG1_AAS
(SEQ ID NO: 6)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLGGGSGGGSGGGSNPQIAAHVISEASSKTTS
VLQWAEKGYYTMSNNLVTLENGKQLTVKRQGLYYIYAQVTFCSNREASSQAPFIAS
LCLKSPGRFERILLRAANTHSSAKPCGQQSIHLGGVFELQPGASVFVNVTDPSQVSHG
TGFTSFGLLKLGGGSGGGSGGGSNPQIAAHVISEASSKTTSVLQWAEKGYYTMSNNL
VTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKLGGGGSE
PKSSDKTHTCPPCPAPEAAGGPSVFLFPPKPKDTLMISRTPEVTCVVVSVSHEDP
EVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEA
LHNHYTQKSLSLSPGK
>TPP000182993|C4LW26|mature_protein|NPQ_G3S3_sthCD40L_IgG1_AAS
(SEQ ID NO: 7)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLGGGSGGGSGGGSNPQIAAHVISEASSKTTS
VLQWAEKGYYTMSNNLVTLENGKQLTVKRQGLYYIYAQVTFCSNREASSQAPFIAS
LCLKSPGRFERILLRAANTHSSAKPCGQQSIHLGGVFELQPGASVFVNVTDPSQVSHG
TGFTSFGLLKLGGGSGGGSGGGSNPQIAAHVISEASSKTTSVLQWAEKGYYTMSNNL
VTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKLEPKSSDK
THTCPPCPAPEAAGGPSVFLFPPKPKDTLMISRTPEVTCVVVSVSHEDPEVKFNW
YVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYT
QKSLSLSPGK
>TPP000182992|C4LW25|mature_protein|NPQ_sthCD40L_G4S2_IgG1_AAS
(SEQ ID NO: 8)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLEGKSSGSGSNPQIAAHVISEASSKTTSVLQ
WAEKGYYTMSNNLVTLENGKQLTVKRQGLYYIYAQVTFCSNREASSQAPFIASLCL
KSPGRFERILLRAANTHSSAKPCGQQSIHLGGVFELQPGASVFVNVTDPSQVSHGTGF
TSFGLLKLEGKSSGSGSNPQIAAHVISEASSKTTSVLQWAEKGYYTMSNNLVTLENG
KQLTVKRQGLYYIYAQVTFCSNREASSQAPFIASLCLKSPGRFERILLRAANTHSSAK
PCGQQSIHLGGVFELQPGASVFVNVTDPSQVSHGTGFTSFGLLKLGGGGSGGGGSE
PKSSDKTHTCPPCPAPEAAGGPSVFLFPPKPKDTLMISRTPEVTCVVVSVSHEDP
EVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEA
LHNHYTQKSLSLSPGK
>TPP000182991|C4LW24|mature_protein|NPQ_sthCD40L_G4S3_IgG1_AAS
(SEQ ID NO: 9)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLEGKSSGSGSNPQIAAHVISEASSKTTSVLQ
WAEKGYYTMSNNLVTLENGKQLTVKRQGLYYIYAQVTFCSNREASSQAPFIASLCL
KSPGRFERILLRAANTHSSAKPCGQQSIHLGGVFELQPGASVFVNVTDPSQVSHGTGF
TSFGLLKLEGKSSGSGSNPQIAAHVISEASSKTTSVLQWAEKGYYTMSNNLVTLENG
KQLTVKRQGLYYIYAQVTFCSNREASSQAPFIASLCLKSPGRFERILLRAANTHSSAK
PCGQQSIHLGGVFELQPGASVFVNVTDPSQVSHGTGFTSFGLLKLGGGGSGGGGSG
GGGSEPKSSDKTHTCPPCPAPEAAGGPSVFLFPPKPKDTLMISRTPEVTCVVVSV
SHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGK
>TPP000182990|C4LW23|mature_protein|NPQ_sthCD40L_G4S4_IgG1_AAS
(SEQ ID NO: 10)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLEGKSSGSGSNPQIAAHVISEASSKTTSVLQ
WAEKGYYTMSNNLVTLENGKQLTVKRQGLYYIYAQVTFCSNREASSQAPFIASLCL
KSPGRFERILLRAANTHSSAKPCGQQSIHLGGVFELQPGASVFVNVTDPSQVSHGTGF
TSFGLLKLEGKSSGSGSNPQIAAHVISEASSKTTSVLQWAEKGYYTMSNNLVTLENG
KQLTVKRQGLYYIYAQVTFCSNREASSQAPFIASLCLKSPGRFERILLRAANTHSSAK
PCGQQSIHLGGVFELQPGASVFVNVTDPSQVSHGTGFTSFGLLKLGGGGSGGGGS
GGGGSGGGGSEPKSSDKTHTCPPCPAPEAAGGPSVFLFPPKPKDTLMISRTPEVT
CVVVSVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQG
NVFSCSVMHEALHNHYTQKSLSLSPGK
>TPP000182989|C4LW22|mature_protein|NPQ_sthCD40L_G4S_IgG1_AAS
(SEQ ID NO: 11)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLEGKSSGSGSNPQIAAHVISEASSKTTSVLQ
WAEKGYYTMSNNLVTLENGKQLTVKRQGLYYIYAQVTFCSNREASSQAPFIASLCL
KSPGRFERILLRAANTHSSAKPCGQQSIHLGGVFELQPGASVFVNVTDPSQVSHGTGF
TSFGLLKLEGKSSGSGSNPQIAAHVISEASSKTTSVLQWAEKGYYTMSNNLVTLENG
KQLTVKRQGLYYIYAQVTFCSNREASSQAPFIASLCLKSPGRFERILLRAANTHSSAK
PCGQQSIHLGGVFELQPGASVFVNVTDPSQVSHGTGFTSFGLLKLGGGGSEPKSSDK
THTCPPCPAPEAAGGPSVFLFPPKPKDTLMISRTPEVTCVVVSVSHEDPEVKFNW
YVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYT
QKSLSLSPGK
>TPP000182988|C4LW21|mature_protein|NPQ_sthCD40L_IgG1_AAS
(SEQ ID NO: 12)
NPQIAAHVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIHLGGVFELQP
GASVFVNVTDPSQVSHGTGFTSFGLLKLEGKSSGSGSNPQIAAHVISEASSKTTSVLQ
WAEKGYYTMSNNLVTLENGKQLTVKRQGLYYIYAQVTFCSNREASSQAPFIASLCL
KSPGRFERILLRAANTHSSAKPCGQQSIHLGGVFELQPGASVFVNVTDPSQVSHGTGF
TSFGLLKLEGKSSGSGSNPQIAAHVISEASSKTTSVLQWAEKGYYTMSNNLVTLENG
KQLTVKRQGLYYIYAQVTFCSNREASSQAPFIASLCLKSPGRFERILLRAANTHSSAK
PCGQQSIHLGGVFELQPGASVFVNVTDPSQVSHGTGFTSFGLLKLEPKSSDKTHTCP
PCPAPEAAGGPSVFLFPPKPKDTLMISRTPEVTCVVVSVSHEDPEVKFNWYVDG
VEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSL
SLSPGK
Font Scheme: HUMAN IgG Fc-TETHER CD40L-LINKER-CD40L-LINKER-CD40L
>TPP000182986|C4LW19|mature_protein|IgG1_AAS_G4S2_NPQ_sthCD40L
(SEQ ID NO: 13)
HTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGGGGGSGGGGSNPQIAAHVISEASSKTTSVLQWAEKGYYTMSNNLVTLE
NGKQLTVKRQGLYYIYAQVTFCSNREASSQAPFIASLCLKSPGRFERILLRAANTHSS
AKPCGQQSIHLGGVFELQPGASVFVNVTDPSQVSHGTGFTSFGLLKLEGKSSGSGSNP
QIAAHVISEASSKTTSVLQWAEKGYYTMSNNLVTLENGKQLTVKRQGLYYIYAQVT
FCSNREASSQAPFIASLCLKSPGRFERILLRAANTHSSAKPCGQQSIHLGGVFELQPGA
SVFVNVTDPSQVSHGTGFTSFGLLKLEGKSSGSGSNPQIAAHVISEASSKTTSVLQWA
EKGYYTMSNNLVTLENGKQLTVKRQGLYYIYAQVTFCSNREASSQAPFIASLCLKSP
GRFERILLRAANTHSSAKPCGQQSIHLGGVFELQPGASVFVNVTDPSQVSHGTGFTSF
GLLKL
>TPP000182985|C4LW18|mature_protein|IgG1_AAS_G4S3_NPQ_sthCD40L
(SEQ ID NO: 14)
HTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGGGGGSGGGGSGGGGSNPQIAAHVISEASSKTTSVLQWAEKGYYTMSN
NLVTLENGKQLTVKRQGLYYIYAQVTFCSNREASSQAPFIASLCLKSPGRFERILLRA
ANTHSSAKPCGQQSIHLGGVFELQPGASVFVNVTDPSQVSHGTGFTSFGLLKLEGKSS
GSGSNPQIAAHVISEASSKTTSVLQWAEKGYYTMSNNLVTLENGKQLTVKRQGLYYI
YAQVTFCSNREASSQAPFIASLCLKSPGRFERILLRAANTHSSAKPCGQQSIHLGGVFE
LQPGASVFVNVTDPSQVSHGTGFTSFGLLKLEGKSSGSGSNPQIAAHVISEASSKTTSV
LQWAEKGYYTMSNNLVTLENGKQLTVKRQGLYYIYAQVTFCSNREASSQAPFIASL
CLKSPGRFERILLRAANTHSSAKPCGQQSIHLGGVFELQPGASVFVNVTDPSQVSHGT
GFTSFGLLKL
>TPP000182984|C4LW17|mature_protein|IgG1_AAS_G4S4_NPQ_sthCD40L
(SEQ ID NO: 15)
HTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGGGGGSGGGGSGGGGSGGGGSNPQIAAHVISEASSKTTSVLQWAEKGY
YTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLK
LEGKSSGSGSNPQIAAHVISEASSKTTSVLQWAEKGYYTMSNNLVTLENGKQLTVKR
QGLYYIYAQVTFCSNREASSQAPFIASLCLKSPGRFERILLRAANTHSSAKPCGQQSIH
LGGVFELQPGASVFVNVTDPSQVSHGTGFTSFGLLKLEGKSSGSGSNPQIAAHVISEA
SSKTTSVLQWAEKGYYTMSNNLVTLENGKQLTVKRQGLYYIYAQVTFCSNREASSQ
APFIASLCLKSPGRFERILLRAANTHSSAKPCGQQSIHLGGVFELQPGASVFVNVTDPS
QVSHGTGFTSFGLLKL
>TPP000182983|C4LW16|mature_protein|IgG1_AAS_G4S_NPQ_G3S3_sthCD40L
(SEQ ID NO: 16)
HTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGGGGGSNPQIAAHVISEASSKTTSVLQWAEKGYYTMSNNLVTLENGKQL
TVKRQGLYYIYAQVTFCSNREASSQAPFIASLCLKSPGRFERILLRAANTHSSAKPCG
QQSIHLGGVFELQPGASVFVNVTDPSQVSHGTGFTSFGLLKLGGGSGGGSGGGSNPQI
AAHVISEASSKTTSVLQWAEKGYYTMSNNLVTLENGKQLTVKRQGLYYIYAQVTFC
SNREASSQAPFIASLCLKSPGRFERILLRAANTHSSAKPCGQQSIHLGGVFELQPGASV
FVNVTDPSQVSHGTGFTSFGLLKLGGGSGGGSGGGSNPQIAAHVISEASSKTTSVLQ
WAEKGYYTMSNNLVTLENGKQLTVKRQGLYYIYAQVTFCSNREASSQAPFIASLCL
KSPGRFERILLRAANTHSSAKPCGQQSIHLGGVFELQPGASVFVNVTDPSQVSHGTGF
TSFGLLKL
>TPP000182982|C4LW15|mature_protein|IgG1_AAS_G4S2_NPQ_G3S3_sthCD40L
(SEQ ID NO: 17)
HTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGGGGGSGGGGSNPQIAAHVISEASSKTTSVLQWAEKGYYTMSNNLVTLE
NGKQLTVKRQGLYYIYAQVTFCSNREASSQAPFIASLCLKSPGRFERILLRAANTHSS
AKPCGQQSIHLGGVFELQPGASVFVNVTDPSQVSHGTGFTSFGLLKLGGGSGGGSGG
GSNPQIAAHVISEASSKTTSVLQWAEKGYYTMSNNLVTLENGKQLTVKRQGLYYIY
AQVTFCSNREASSQAPFIASLCLKSPGRFERILLRAANTHSSAKPCGQQSIHLGGVFEL
QPGASVFVNVTDPSQVSHGTGFTSFGLLKLGGGSGGGSGGGSNPQIAAHVISEASSKT
TSVLQWAEKGYYTMSNNLVTLENGKQLTVKRQGLYYIYAQVTFCSNREASSQAPFI
ASLCLKSPGRFERILLRAANTHSSAKPCGQQSIHLGGVFELQPGASVFVNVTDPSQVS
HGTGFTSFGLLKL
>TPP000182981|C4LW14|mature_protein|IgG1_AAS_G4S3_NPQ_G3S3_sthCD40L
(SEQ ID NO:18)
HTCPPCPAPEAAGGPSVFLFPPKPKDTLMISRTPEVTCVVVSVSHEDPEVKFNWYVD
GVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKS
LSLSPGGGGGSGGGGSGGGGSNPQIAAHVISEASSKTTSVLQWAEKGYYTMSNNL
VTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKLGGGSGG
GSGGGSNPQIAAHVISEASSKTTSVLQWAEKGYYTMSNNLVTLENGKQLTVKRQGL
YYIYAQVTFCSNREASSQAPFIASLCLKSPGRFERILLRAANTHSSAKPCGQQSIHLGG
VFELQPGASVFVNVTDPSQVSHGTGFTSFGLLKLGGGSGGGSGGGSNPQIAAHVISEA
SSKTTSVLQWAEKGYYTMSNNLVTLENGKQLTVKRQGLYYIYAQVTFCSNREASSQ
APFIASLCLKSPGRFERILLRAANTHSSAKPCGQQSIHLGGVFELQPGASVFVNVTDPS
QVSHGTGFTSFGLLKL
>TPP000182980|C4LW13|mature_protein|IgG1_AAS_G4S4_NPQ_G3S3_sthCD40L
(SEQ ID NO: 19)
HTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGGGGGSGGGGSGGGGSGGGGSNPQIAAHVISEASSKTTSVLQWAEKGY
YTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLK
LGGGSGGGSGGGSNPQIAAHVISEASSKTTSVLQWAEKGYYTMSNNLVTLENGKQL
TVKRQGLYYIYAQVTFCSNREASSQAPFIASLCLKSPGRFERILLRAANTHSSAKPCG
QQSIHLGGVFELQPGASVFVNVTDPSQVSHGTGFTSFGLLKLGGGSGGGSGGGSNPQI
AAHVISEASSKTTSVLQWAEKGYYTMSNNLVTLENGKQLTVKRQGLYYIYAQVTFC
SNREASSQAPFIASLCLKSPGRFERILLRAANTHSSAKPCGQQSIHLGGVFELQPGASV
FVNVTDPSQVSHGTGFTSFGLLKL
>TPP000161222|C40W33|mature_protein|JNJ75347415
(SEQ ID NO: 32)
IAAHVISEASSKTTSVLQWAEKGYYTMSNNLVTLENGKQLTVKRQGLYYIYAQVTF
CSNREASSQAPFIASLCLKSPGRFERILLRAANTHSSAKPCGQQSIHLGGVFELQPGAS
VFVNVTDPSQVSHGTGFTSFGLLKLEGKSSGSGSQIAAHVISEASSKTTSVLQWAEKG
YYTMSNNLVTLENGKQLTVKRQGLYYIYAQVTFCSNREASSQAPFIASLCLKSPGRF
ERILLRAANTHSSAKPCGQQSIHLGGVFELQPGASVFVNVTDPSQVSHGTGFTSFGLL
KLEGKSSGSGSQIAAHVISEASSKTTSVLQWAEKGYYTMSNNLVTLENGKQLTVKR
QGLYYIYAQVTFCSNREASSQAPFIASLCLKSPGRFERILLRAANTHSSAKPCGQQSIH
LGGVFELQPGASVFVNVTDPSQVSHGTGFTSFGLLKLVERKSCVECPPCPAPPVAG
PSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK

### Example 3: HEK-Blue Assay

To ensure the molecules were functionally active CD40 agonists, the ability of the trimer fusion proteins to activate native CD40 receptor using the HEK-Blue reporter cell line (Invivogen) was assayed.

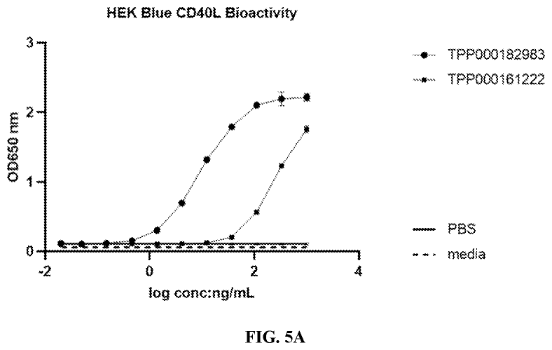

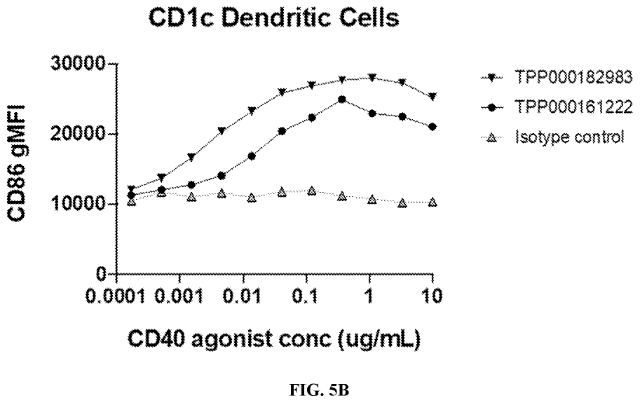

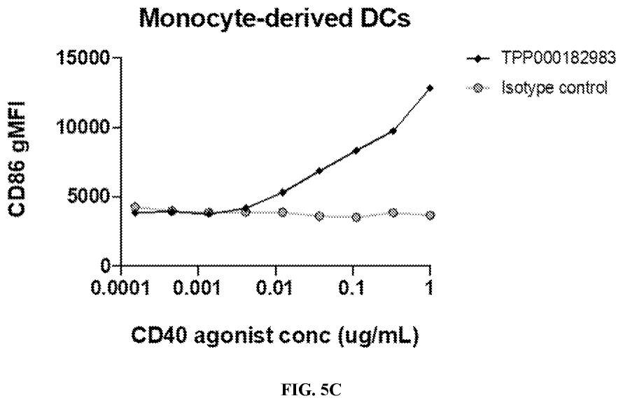

HEK-Blue CD40L cells from Invivogen were cultured in growth media containing DMEM, 2 mM GlutaMAX, 100% heat-inactivated FBS, 100 ug/mL of Normocin and 100 ug/mL of Pen-Strep. Cell were prepared at 2.8×10⁵ cells/ml in test media (DMVEM+10% heat inactivated FBS+100 ug/mL PenStrep) for assay set up. 180 μl of cell suspension was added to 96 well plate for a final density of 5×10⁴ cells/well. 20 uL of test article was added to each well for a total volume of 200 μL per well. Plates were incubated at 37° C. in a CO₂ incubator for 20-24 h. QUANTI-Blue solution from Invivogen was prepared according to the manufacturer's protocol and 180 μl of solution was added to each well of 96-well flat-bottom plate. 20 uL of induced HEK-Blue CD40L cell supernatant was added to assay plate and incubated at 37° C. SLAP levels were determined using spectrophotometer (635 nm). Functional activity of the CD40L trimer tested using HEK-Blue reporter assay is shown in FIG. 5A. Results of monocyte derived dendritic cell activation assay are shown in FIG. 5B. Results of the mature dendritic cell activation assay without cross linking antibody treatment are shown in FIG. 5C.

TPP000182983 showed a 100 fold increase in activity when compared to the original molecule TPP000161222 using the HEK-Blue reporter assay (FIG. 5A).

### Example 4: MSD Electro-Chemiluminescence Detection Assay

Binding properties of the engineered molecules was compared using Meso Scale Discovery bioluminescence assay (MSD) assay to measure the binding of the fusion proteins to recombinant CD40 receptor.

Streptavidin sensor plates were coated with 10 ug/ml of biotinylated CD40 overnight at 4° C. The plates were washed 3× with PBS with 0.05% Tween 20 (PBST). Three-fold dilutions series of the test articles were prepared in PBS with 0.2% bovine serum albumin and 50 ul was applied to the plates for 1 hour at RT with shaking. Plates were washed 3× with PBST and anti-human Fc detection reagent at 2 ug/ml final concentration was applied for 1 hour at RT with shaking. The plates were washed 3× with PBST and 150 ul of 1× read buffer was added to the plates and data was collected on MSD instrument. Data was imported into GRAPHPAD Prism 8 software and analyzed and plotted based on the results three separate experiments.

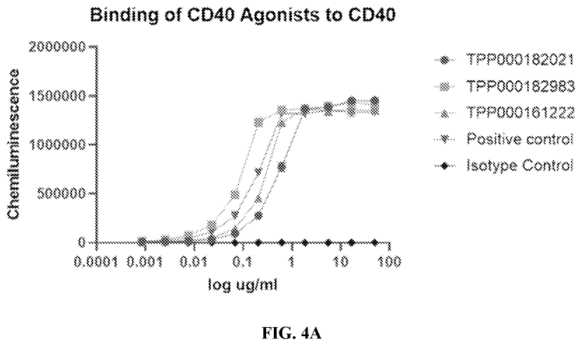

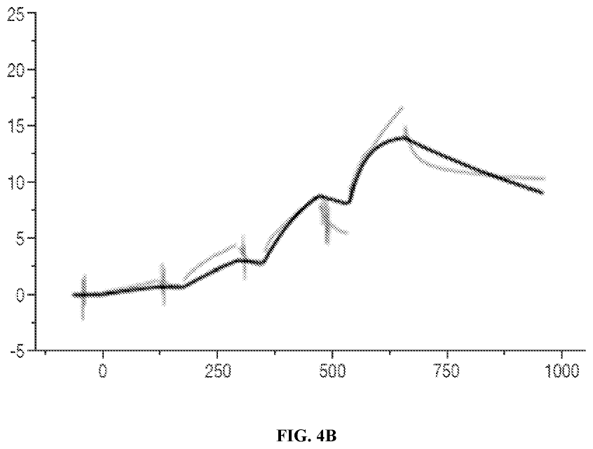

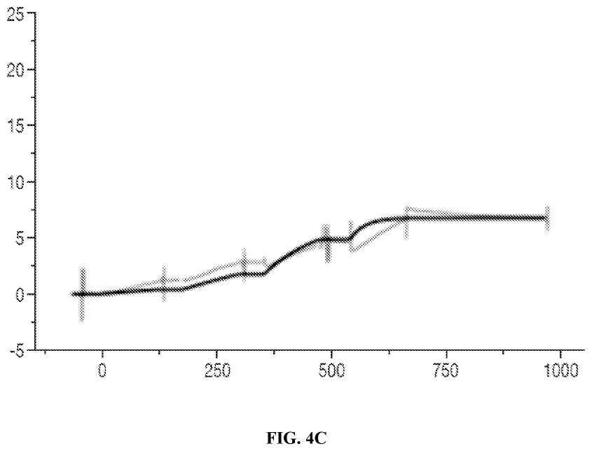

The newly designed trimers showed increased binding compared to the original designed trimer using both MSD and SPR (described in Examples 4 and 5) (FIGS. 4A-4C).

### Example 5: Surface Plasmon Resonance Analysis for CD40L

Binding properties of the engineered molecules was compared using surface plasmon resonance (SPR) assay to measure the binding of the fusion proteins to recombinant CD40 receptor.

Surface plasmon resonance (SPR) with the technology of Biacore requires one interactant to be immobilized or captured on the sensor surface. The other interactant flows over the modified sensor surface. The interaction of the two proteins occurring on the sensor surface is monitored in real-time, by measuring the change in the instrument response. SPR experiments for CD40L were performed using a Biacore 8K optical biosensor (GE Healthcare). The samples were prepared in HBS buffer containing 0.05% P20 and the experiments performed at 25° C. using a C1 sensor chip. The chip was pretreated with 100 mM Gly pH12-0.3% Triton and this was followed by covalent immobilization of a goat anti-human Fe antibody. A goat anti-human IgG Fc fragment specific antibody was diluted in 10 mM sodium acetate buffer pH 4.5 and ˜600 response units (RU) were coupled to the carboxymethylated dextran surface of the C1 chip using amine-coupling chemistry. The remaining reactive groups on the surface were deactivated using ethanolamine-HCl. To perform the binding experiments, >50 response units (RU) of CD40L-Fc fusion constructs were captured and followed by injections of hu CD40 at concentrations from 4.7 nM to 300 nM at 50 μL/min. The association phase was monitored for 3 minutes, and the dissociation phase was monitored for 10 minutes. The sensor chip surface was regenerated by injections of 0.85% H₃PO₄. A flow cell free of CD40L constructs was used as a reference. Data were processed using the Insight software, version 2 (GE healthcare). Double reference subtraction of the data was performed to correct for buffer contribution to the signal and instrument noise.

Interestingly, the newly designed trimers showed increased binding compared to the original designed trimer using both MSD and SPR (FIGS. 4A-4C).

### Example 6: CDc+ Dendritic Cell Activation Assay

To assess the ability of the trimer fusions to activate human derived immune cells, a CMV recall assay (Example 7, below) and a dendritic cell cross presentation assay were employed.

Pre-sorted CD1c+ dendritic cells were thawed and then incubated overnight in a 37° C. incubator at 2×10⁶ cells/mL in complete RPMI 1640 (10% FBS, L-Glutamine, Non-essential amino acids, Sodium Pyruvate, and pen/strep) containing 80 ng/mL GM-CSF and 80 ng/mL IL-4 (PeproTech, Inc). The following day, cells were washed with complete RPMI and plated at 1×10⁵ cells/well in 96-well round bottom plates. Dendritic cells were activated with titrating concentrations of selected CD40 agonist molecules and incubate for 24 hrs at 37° C. Cells were washed and flow cytometric analysis was performed for extracellular dendritic cell activation markers.

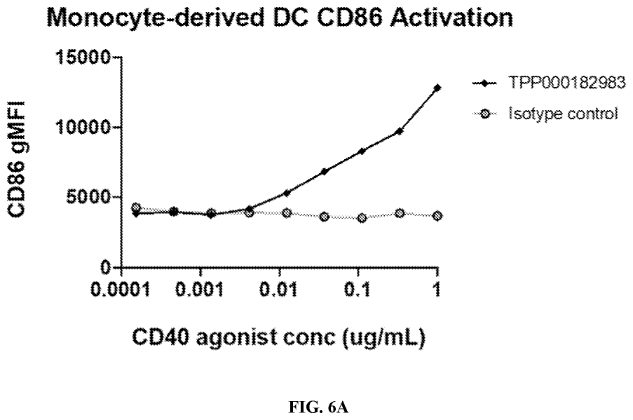

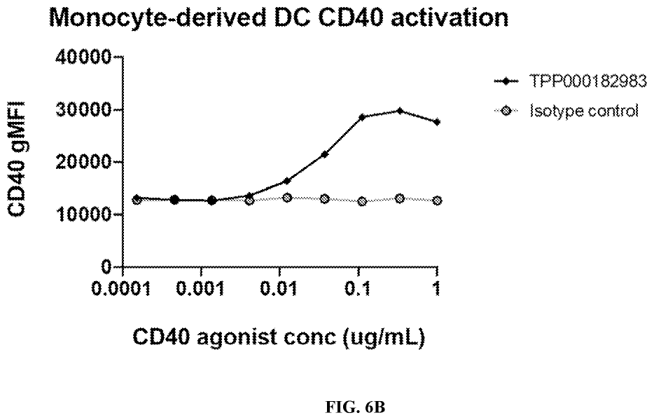

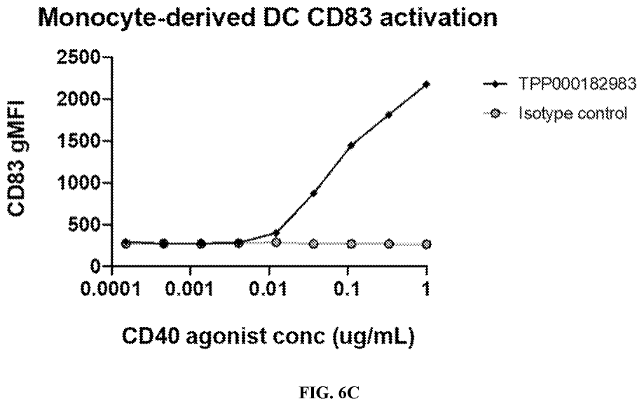

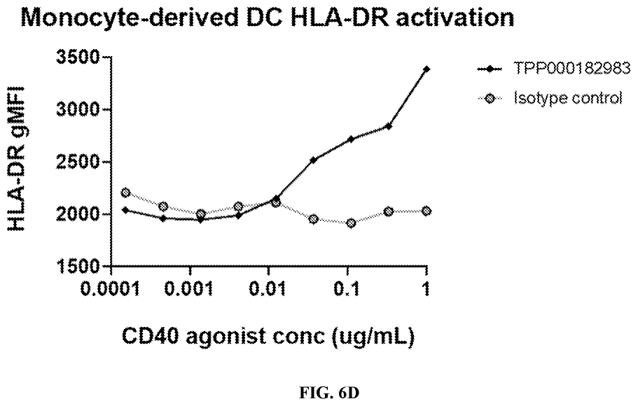

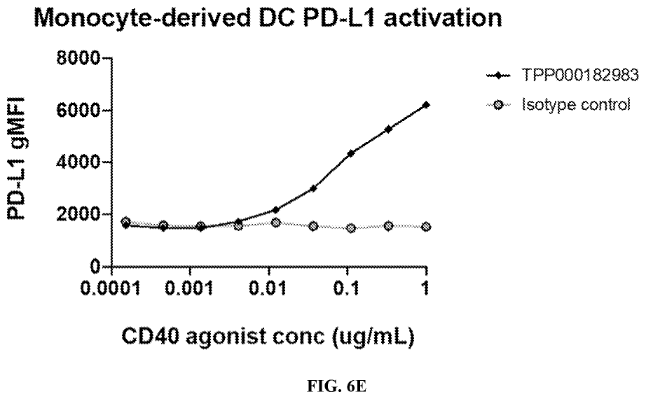

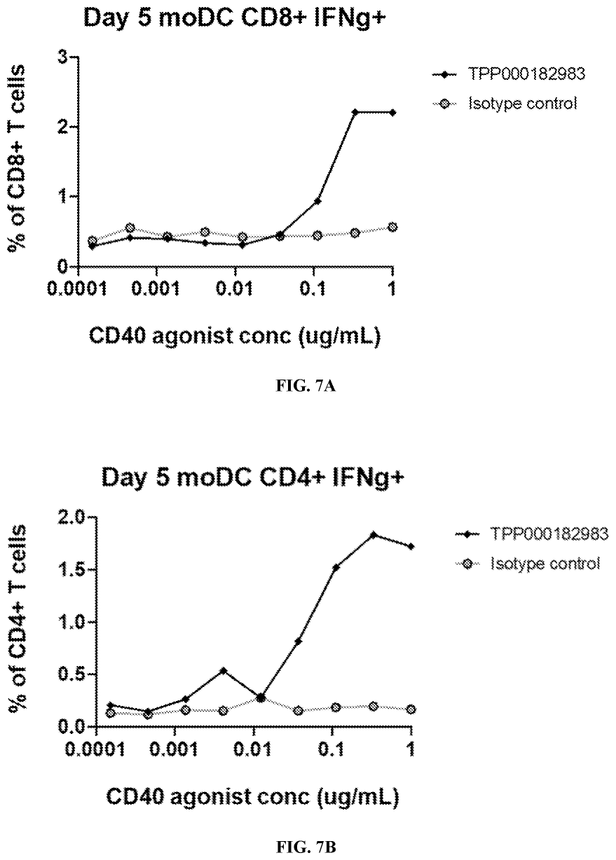

The relative shifts of cell surface markers, mainly CD80 and CD86, were measured in the presence and absence of trimer fusion proteins. Surprisingly, TPP000182983 showed increased DC activation and T-cell activation (Example 7, below) (FIGS. 6A-6E and FIGS. 7A-7B) when compared to other known CD40L agonist trimers.

To test the hypothesis that a CD40L trimer fusion protein had activity by itself, (i.e., without forming a dimer of two copies of the CD40L trimer fusion proteins through the interaction between their Fc portions), molecules that contained a CD40L trimer fused to a Fc monomer peptide with mutations in the CH3 domain that abrogated dimer formation were designed and constructed (“Mono Fc” molecules, see e.g., FIG. 1B). These Mono-Fc molecules were tested in a 24-hour human DC activation assay with two separate donors. Upregulation of CD86, a marker for APC activation, was observed at a level similar to that of wild-type CD40L (data not shown), demonstrating that the CD40L trimer alone is active.

### Example 7: T Cell Activation Assay Using Monocyte-Derived Dendritic Cells

Pre-sorted monocytes were thawed then differentiated into monocyte-derived dendritic cells in a 37° C. incubator for 5 days at 2×10⁶ cells/mL in complete RPMI 1640 (10% FBS, L-Glutamine, Non-essential amino acids, Sodium Pyruvate, and pen/strep) containing 80 ng/mL GM-CSF and 80 ng/mL IL-4 (PeproTech, Inc). On day 3, equal volume complete RPMI containing 160 ng/mL GM-CSF and 160 ng/mL IL-4 were added. On day 5, cells were washed with complete RPMI and plated 3×10⁴ cells/well in 96-well round bottom plates. To assess T cell activation using monocyte-derived DCs, titrating amounts of selected CD40 agonist molecules were added, 3×10⁵ matched donor T cells, CEF peptides (Cytomegalovirus, Epstein-Barr virus, and Influenza virus peptides; ImmunoSpot) at a final concentration of 1 ug/mL, and IL-15 at a final concentration of 10 μg/mL (PeproTech, Inc). On day 2, half of the media was replaced with complete RPMI containing 20 μg/mL IL-15 and 20 IU/mL IL-2 (R&D Systems). On day 5, half of the media was replaced with complete RPMI containing 20 ug/mL IL-15, 20 IU/mL IL-2, CEF peptides at a final concentration of 1 ug/mL, CD107a antibody at 1:200 final concentration (Biolegend), and 1× Protein Transport Inhibitor Cocktail (Thermo Fisher). The cells were subsequently incubated overnight in a 37° C. incubator then proceed to flow cytometry analysis for production of intracellular effector cytokines.

Taken together, the examples demonstrate that the newly optimized CD40L trimer, TPP000182983, is a stable and functionally superior CD40 agonist.

### Example 8: Analytical Ultra-Centrifugation

Samples were loaded into centrifuge cells equipped with 1.2 cm Beckman centerpieces (rated to 50K rpm) and quartz windows. The cells are assembled and torqued to 130 lbs. The centrifuge cells were placed into an An-50 (8 hole) or An-60 (4 hole) rotor and placed within the Beckman Optima AUC chamber. The temperature of the AUC was equilibrated to 20.5° C. for at least one hour with the rotor in the chamber before initiating the run. Runs were performed at 40K rpm for mAb sample with scan count (250 scans), frequency of scan collection (90 seconds), data resolution (10 μM). Absorbance data was collected at 280 nm. Initially the data were analyzed using the software program DCDT (Philo 2006) in order to determine the meniscus position and to observe the sedimentation distribution profiles. The data were then analyzed using the direct boundary fitting software (Stafford and Sherwood, Biophys Chem 108(1-3): 231-243 (2004)). The meniscus position was determined by DCDT+, the baseline was set at 7.2, and manually choosing the fit range. A two species, non-interacting model was used to fit the data with the first species corresponding to monomer and the second species corresponding to dimer.

### Example 9: Antitumor Activity

To access the in vivo antitumor activity of the engineered molecules, colon adenocarcinoma tumor growth was evaluated in a human CD40 knock-in mouse model treated with the engineered molecule TPP000182983 (C4LW16). The dosage amount and regimen for the TPP000182983 was selected to mimic expression of encoded protein from gene delivery platforms (e.g., in the form encoded by mRNA, adeno-associated viruses, or oncolytic viruses, etc.) and are considered sub-optimal in comparison to recombinant protein therapy.

Human CD40 knock-in mice were implanted with 5×10⁵ MC38 5AG cells. When tumor volumes reached approximately 100 mm³, mice were randomized into treatment and control groups. Mice were then administered 20 μg of either TPP000182983 or a negative control isotype antibody intravenously on Days 1, 4, and 6 following randomization and tumor growth was measured over time.

During the treatment period, TPP000182983 demonstrated strong antitumor activity with tumor growth inhibition of 49.99% in comparison to the isotype control and a p value of 0.036 (data not shown).

These results demonstrate that the engineered trimeric CD40L fusion proteins can effectively inhibit tumor growth, even at a sub-optimal low dosage.

### Example 10: Viral Clearance

The ability of engineered molecules to enhance viral clearance is evaluated in a human CD40 knock-in mouse model. A group of 6- to 12-week old female mice are injected intravenously with 1×10⁷ pfu influenza virus through the tail veins. The inoculated mice are randomized into treatment and control groups. Mice in the treatment group are then infused intravenously with a polypeptide comprising the single chain trimeric CD40L fusion protein according to the present disclosure. Mice in the control group are infused with PBS or a negative control isotype antibody. Viral titers in the mice are measured overtime.

During the treatment period, mice administered with the engineered trimeric CD40L fusion protein have lower viral titers and faster viral clearance than mice administered the negative control isotype antibody or PBS.

These results are to demonstrate that the engineered trimeric CD40L fusion proteins according to the present disclosure effectively enhance clearance of the influenza virus.

### Example 11: Vaccine Adjuvant

Engineered trimeric CD40L fusion proteins are evaluated for their ability to enhance the immune response to a vaccine composition in human CD40 knock-in mice.

Human CD40 knock-in mice are randomized into groups and are administered a vaccine composition comprising a target viral antigen. A polypeptide comprising the single chain trimeric CD40L fusion protein according to the present disclosure is administered concurrently or sequentially with the vaccine composition to the mice (treatment group). In a control group, the mice are administered with PBS in lieu of the engineered trimeric CD40L fusion protein. The immune response of the mice to the viral antigen in the treatment group and control group are monitored over time. The results show that mice in the treatment group exhibited significantly higher immune responses towards the viral antigen, comparing to mice in the control group. Particularly, titers of antibodies specifically binding to the target viral antigen are significantly higher, and concentration of antibody-producing plasma cells in mice of the treatment group is significantly higher in the mice of the treatment group comparing to control.

Mice in the treatment and control groups are sacrificed at the end of observation period, and dissected tissues known to be infected by the virus (e.g., lungs) are isolated and prepared for further analysis. In an in situ immunohistochemistry study, a cell-specific marker for dendritic cells is stained using Alexa™ Fluor 488, thus visualizing dendritic cells in the tissue sample as green dots under fluorescent microscopy. The target viral antigen used in the vaccine composition is stained with Alexa™ Fluor 568, visualizing the antigen molecules as red dots. Dendritic cells presenting the viral antigen are visualized as co-localization of the two fluorescent signals. The total number of dendritic cells in the tissue sample and the percentage of dendritic cells presenting the viral antigen are measured using fluorescent microscopy. The results show a significant increase in the percentage of matured dendritic cells presenting the viral antigen in mice that receive the vaccine composition together with the engineered trimeric CD40L fusion protein (treatment group), comparing to mice receiving only the vaccine composition (control group).

To further evaluate the effect of the engineered trimeric CD40L fusion protein on presentation of the viral antigen by antigen presenting cells, immunoprecipitation studies are performed to measure association between the viral antigen and MHC complex. A homogenate of the dissected lung tissue from sacrificed mice are prepared according to standard protocols. The homogenate is incubated with beads coated with a monoclonal antibody that specifically bind to Class I MHC or Class II MHC II molecules under a suitable condition to allow association of the antibody with its target protein and protein complexes in the tissue sample. The beads are then separated from the tissue by centrifugation, pulling down proteins and protein complexes associated with coated antibody together. The pull-downed protein sample are then processed for analyzing its contents by western blots. A monoclonal antibody specifically for the target viral antigen is used. The results show a significant increase in the amount of viral antigen immunoprecipitated with the MHC molecules (both Class I and Class II molecules examined in this study) from samples isolated from mice that receive the vaccine composition together with the engineered trimeric CD40L fusion protein (treatment group), comparing to mice receiving only the vaccine composition (control group).

These results are to demonstrate that the engineered trimeric CD40L fusion proteins according to the present disclosure effectively enhance immune responses to the vaccine when co-administered as an adjuvant with the vaccine. The increased immune response can be attributed to at least the increased presentation of the viral antigen by antigen presenting cells (such as dendritic cells) in the immunized animal.

To monitor effects of the engineered trimeric CD40L fusion proteins on long-term immune memory, mice are administered with an initial dose of the vaccine composition in combination with a polypeptide comprising the trimeric CD40L fusion protein according to the present disclosure on Day 0 (treatment group). Then the mice are split into a first group of boosted mice and a second group of un-boosted mice, where the boosted mice are given a second administration of the vaccine composition on Day 7, while the un-boosted mice are given PBS on day 7. In a control group, mice are administered with the vaccine composition alone on day 0, and separated into the boosted group and un-boosted group to receive a second dose of the vaccine composition or PBS, respectively, on Day 7. The immune response of the mice to the viral antigen in the treatment group and control group are monitored over time.

The results are to show that mice in both of the treatment group and control group produce antibodies specifically binding to the target viral antigen after receiving the initial dose of the vaccine composition. For mice in the treatment group, the antibody titer in unboosted mice remains high and comparable to antibody titers in the boosted mice on Day 14 and Day 28. For mice in the control group, the antibody titers in the unboosted mice are significantly lower than the boosted mice on Day 28. Mice are sacrificed on Day 28, and it is observed that mice in the control group develops memory B cells, while memory B cell formation is not observed from the control group mice (boosted or unboosted).

These results are to indicate that the engineered trimeric CD40L fusion proteins according to the present disclosure effectively enhance humoral immune responses and antibody production targeting a viral antigen, and promotes faster formation of immune memory against the virus. The engineered trimeric CD40L fusion proteins according to the present disclosure can be used as an adjuvant of a vaccine composition specifically tailored for the prevention of the target virus infection in a subject.

### Example 12: Fc Monomer Peptide Activity

An Fc monomer peptide is designed to test the hypothesis that the Fc monomer has activity. The Fc monomer is tested in a 24-hour human DC activation assay with two separate donors. Similar to WT CD40L activity, CD86 is upregulated, which is a marker for APC activation, indicating that the Fc monomer is active.

### Example 13: Prognostic Method for the Determining Suitability of a Treatment

Individuals or subpopulations of patients having cancer or a solid tumor that would benefit from treatment with the single chain trimeric CD40L fusion protein according to the present disclosure can be identified using prognostic assays that look for the presence or absence of, or elevated or diminished levels of, prognostic markers upon administration of the single chain trimeric CD40L fusion protein. Subjects identified as being responsive to treatment with the single chain trimeric CD40L fusion protein according to the present disclosure, can thus be treated. Alternatively, they can be further screened for potential benefits from the single chain trimeric CD40L fusion protein using an ex vivo prognostic assays, for example, to identify whether a cancer or solid tumor would be more responsive to treatment comprising administration of the single chain trimeric CD40L fusion protein according to the present disclosure.

An ex vivo prognostic assay comprises providing a test biological sample and a control biological sample from a candidate subject, where these biological samples comprise CD40-expressing antigen presenting cells that have been stimulated with single chain trimeric CD40L fusion protein, either in vivo or ex vivo; detecting the expression level of at least one biomarker within the test biological sample; and comparing the expression level of the biomarker(s) with the corresponding expression level detected in the control biological sample that has not been contacted with the single chain trimeric CD40L fusion protein. Biomarkers for use in these ex vivo prognostic assays include proteins and/or genes whose expression levels are prognostic indicators of responsiveness to treatment intervention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A polypeptide comprising a single chain trimeric CD40 ligand (CD40L) fusion protein, wherein the single chain trimeric CD40L fusion protein comprises three CD40L subunits covalently linked to one another by peptide linkers (CD40L trimer),
optionally wherein the CD40L subunits comprise a portion of the CD40L extracellular domain.

2. The polypeptide of claim 1, wherein the CD40L subunits comprise any one of the sequences selected from SEQ ID NOS:20 to 22, or a fragment thereof.

3. The polypeptide of claim 1, wherein at least one of the peptide linkers is selected from the group consisting of EGKSSGSGS (SEQ ID NO:23) and (G₃S)₃ (SEQ ID NO:25);
optionally wherein at least two of the peptide linkers have the same sequence.

4. The polypeptide of claim 1, wherein the single chain trimeric CD40L fusion protein comprises any one sequence selected from SEQ ID NOS:35-38, or a fragment thereof.

5. The polypeptide of claim 1, wherein the single chain trimeric CD40L fusion protein is fused with a peptide or polypeptide not derived from CD40L.

6. The polypeptide of claim 1, wherein the single chain trimeric CD40L fusion protein is fused to a peptide tether;
optionally wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28); and/or
optionally wherein the peptide tether is fused to the N-terminus of the single chain trimeric CD40L fusion protein or the C-terminus of the single chain trimeric CD40L fusion protein.

7. The polypeptide of claim 1, wherein the single chain trimeric CD40L fusion protein is fused to an Fc monomer peptide.

8. The polypeptide of claim 7, wherein the Fc monomer peptide comprises a human Fc sequence;
optionally wherein the human Fc sequence comprises a sequence selected from immunoglobulins IgG, IgA, IgM, IgD and IgE,
optionally wherein the IgG sequence is selected from IgG1, IgG2, IgG3 and IgG4,
optionally wherein
(i) the IgG sequence comprises an IgG1 sequence, and the IgG1 sequence comprises SEQ ID NOS:30 or 31, or a fragment thereof, or
(ii) the IgG sequence comprises an IgG2 sequence, and the IgG2 sequence comprises SEQ ID NO:29 or a fragment thereof.

9. The polypeptide of claim 7, wherein the single chain trimeric CD40L fusion protein is fused to the Fc monomer peptide via a peptide tether;
optionally wherein the peptide tether comprises from 0 to 20 amino acids;
optionally wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).

10. The polypeptide of claim 7, wherein the CD40L trimer is connected to:
(a) the N-terminus of the Fc monomer peptide,
optionally wherein the single chain trimeric CD40L Fc fusion protein comprises any one sequence selected from SEQ ID NOS:1-12, or a fragment thereof; or
(b) the C-terminus of the Fc monomer peptide,
optionally wherein the single chain trimeric CD40L Fc fusion protein comprises any one sequence selected from SEQ ID NOS:13-19, or a fragment thereof.

11. The polypeptide of claim 1, wherein the single chain trimeric CD40L fusion protein:
(a) enhances activation of a CD40 polypeptide compared to wild-type CD40L;
optionally wherein the activation of the CD40 polypeptide comprises
(i) enhanced immune-stimulatory functions of T cells and/or B cells;
(ii) enhanced activation of B cells, CD4+ T cells, CD8+ T cells, dendritic cells, macrophages, natural killer cells, monocytes, granulocytes, eosinophils and/or neutrophils compared to wild-type CD40L; and/or
(iii) increased expression of the CD40 polypeptide.
(b) enhances anti-tumor activity compared to wild-type CD40L;
(c) enhances pro-inflammatory activity compared to wild-type CD40L;
(d) enhances clearance of an infectious pathogen compared to wild-type CD40L.
(e) increases antibody production by a population of B cells by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%;
(f) increases secretion of a pro-inflammatory cytokine by a population of T cells;
optionally wherein the pro-inflammatory cytokine is IL-1, IL-2, IL-6, IL-12, IL-17, IL-22, IL-23, GM-CSF, TNF-α, IFN-γ, or any combination thereof;
optionally wherein the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%;
(g) increases a minimal percentage of phagocytotic macrophages in a population of macrophages to about 10%, about 20%, about 30%, about 40%, about 45%, about 50%, about 55%, about 60%, about 65%, about 70%, about 75%, about 80%, about 85%, about 90%, about 95%, or about 99%; and/or
(h) increases a minimal percentage of antigen-presenting dendritic cells in a population of dendritic cells to about 10%, about 20%, about 30%, about 40%, about 45%, about 50%, about 55%, about 60%, about 65%, about 70%, about 75%, about 80%, about 85%, about 90%, about 95%, or about 99%.

12. The polypeptide of claim 1, wherein the polypeptide is conjugated to an agent;
optionally wherein the agent is a radioisotope, a metal chelator, an enzyme, a fluorescent compound, a bioluminescent compound, or a chemiluminescent compound.

13. A single chain trimeric CD40L Fc fusion protein comprising (a) three CD40L subunits covalently linked to one another by peptide linkers (CD40L trimer); and (b) an Fc monomer peptide.

14. The single chain trimeric CD40L Fc fusion protein of claim 13, wherein the peptide linker is EGKSSGSGS (SEQ ID NO:23) or (G₃S)₃ (SEQ ID NO:25).

15. The single chain trimeric CD40L Fc fusion protein of claim 113, wherein the Fc monomer peptide is covalently linked to the CD40L trimer by a peptide tether,
optionally wherein the peptide tether comprises between 0 and 20 amino acids, and/or
optionally wherein the peptide tether is selected from the group consisting of (G₄S)₃ (SEQ ID NO:24), (G₄S)₂ (SEQ ID NO:26), (G₄S)₄ (SEQ ID NO:27), and G₄S (SEQ ID NO:28).

16. The single chain trimeric CD40L Fc fusion protein of claim 13, wherein the CD40 ligand subunits comprise a portion of the CD40L extracellular domain.

17. The single chain trimeric CD40L Fc fusion protein of claim 13, wherein the CD40L trimer is connected to:
(a) the N-terminus of the Fc monomer peptide,
optionally wherein the single chain trimeric CD40L Fc fusion protein comprises any one sequence selected from SEQ ID NOS:1-12, or a fragment thereof; or
(b) the C-terminus of the Fc monomer peptide,
optionally wherein the single chain trimeric CD40L Fc fusion protein comprises any one sequence selected from SEQ ID NOS:13-19, or a fragment thereof.

18. The single chain trimeric CD40L Fc fusion protein of claim 13, wherein the CD40 ligand subunits comprise any one of the sequences selected from SEQ ID NOS:20-22, or a fragment thereof.

19. The single chain trimeric CD40L Fc fusion protein of claim 13, wherein the Fc monomer peptide comprises a human Fc sequence,
optionally wherein the human Fc sequence comprises a sequence selected from immunoglobulins IgG, IgA, IgM, IgD and IgE,
optionally wherein the IgG sequence is selected from IgG1, IgG2, IgG3 and IgG4,
optionally wherein
(i) the IgG sequence comprises an IgG1 sequence, and the IgG1 sequence comprises SEQ ID NOS:30 or 31, or a fragment thereof, or
(ii) the IgG sequence comprises an IgG2 sequence, and the IgG2 sequence comprises SEQ ID NO:29 or a fragment thereof.

20. The single chain trimeric CD40L Fc fusion protein of claim 13, wherein the single chain trimeric CD40L Fc fusion protein:
(a) enhances activation of a CD40 polypeptide compared to wild-type CD40L;
optionally wherein the activation of the CD40 polypeptide comprises
(i) enhanced immune-stimulatory functions of T cells and/or B cells;
(ii) enhanced activation of B cells, CD4+ T cells, CD8+ T cells, dendritic cells, macrophages, natural killer cells, monocytes, granulocytes, eosinophils and/or neutrophils compared to wild-type CD40L; and/or
(iii) increased expression of the CD40 polypeptide.
(b) enhances anti-tumor activity compared to wild-type CD40L;
(c) enhances pro-inflammatory activity compared to wild-type CD40L;
(d) enhances clearance of an infectious pathogen compared to wild-type CD40L;
(e) increases antibody production by a population of B cells by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%;
(f) increases secretion of a pro-inflammatory cytokine by a population of T cells;
optionally wherein the pro-inflammatory cytokine is IL-1, IL-2, IL-6, IL-12, IL-17, IL-22, IL-23, GM-CSF, TNF-α, IFN-γ, or any combination thereof;
optionally wherein the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%;
(g) increases a minimal percentage of phagocytotic macrophages in a population of macrophages to about 10%, about 20%, about 30%, about 40%, about 45%, about 50%, about 55%, about 60%, about 65%, about 70%, about 75%, about 80%, about 85%, about 90%, about 95%, or about 99%; and/or
(h) increases a minimal percentage of antigen-presenting dendritic cells in a population of dendritic cells to about 10%, about 20%, about 30%, about 40%, about 45%, about 50%, about 55%, about 60%, about 65%, about 70%, about 75%, about 80%, about 85%, about 90%, about 95%, or about 99%.

21. A dimer comprising two single chain trimeric CD40L Fc fusion proteins of claim 13;
optionally wherein
(i) the dimer is a homodimer; or
(ii) the dimer is formed by association of the Fc monomer peptides.

22. A polynucleotide encoding the polypeptide of claim 1.

23. A vector comprising the polynucleotide of claim 22.

24. A host cell comprising the polynucleotide of claim 22 or the vector of claim 23.

25. A pharmaceutical composition comprising:
(i) a pharmaceutically acceptable carrier, and
(ii) the polypeptide of claim 1.

26. (canceled)

27. A method for producing;
(a) a single chain trimeric CD40L Fc fusion protein or fragment thereof, the method comprising:
(i) introducing into a host cell a polynucleotide encoding the single chain trimeric CD40L Fc fusion protein of claim 8;
(ii) culturing the host cell under conditions to produce the single chain trimeric CD40L Fc fusion protein or fragment thereof, and
(iii) recovering the single chain trimeric CD40L Fc fusion protein or fragment thereof from the cell or culture; or
(b) a dimer comprising two single chain trimeric CD40L Fc fusion proteins, the method comprising:
(i) introducing into a host cell a polynucleotide encoding of the single chain trimeric CD40L Fc fusion protein of claim 8;
(ii) culturing the host cell under conditions to produce the single chain trimeric CD40L Fc fusion protein or fragment thereof;
(iii) recovering the single chain trimeric CD40L Fc fusion protein or fragment thereof from the cell or culture, and
(iv) combining single chain trimeric CD40L Fc fusion proteins or fragments thereof under conditions that favor dimerization; or
(c) a pharmaceutical composition of a single chain trimeric CD40L Fc fusion protein or fragment thereof, the method comprising combining the single chain trimeric CD40L Fc fusion protein of claim 8 or fragment thereof with a pharmaceutically acceptable carrier to obtain the pharmaceutical composition.

28. (canceled)

29. A method of activating:
(a) a CD40 polypeptide, the method comprising:
(i) contacting the CD40 polypeptide with the polypeptide of claim 1, wherein said single chain trimeric CD40L fusion protein activates the CD40 polypeptide upon binding;
(ii) contacting the CD40 polypeptide with the single chain trimeric CD40L Fc fusion protein of claim 13, wherein said single chain trimeric CD40L Fc fusion protein activates the CD40 polypeptide upon binding; or
(iii) contacting the CD40 polypeptide with a dimer comprising two single chain trimeric CD40L Fc fusion proteins of claim 13, wherein said single chain trimeric CD40L Fc fusion protein dimer activates the CD40 polypeptide upon binding;
(b) a target cell, the method comprising:
(i) contacting the target cell with the polypeptide of claim 1, wherein said single chain trimeric CD40L fusion protein activates the target cell upon binding;
(ii) contacting the target cell with the single chain trimeric CD40L Fc fusion protein of claim 13, wherein said single chain trimeric CD40L Fc fusion protein activates the target cell upon binding; or
(iii) contacting the cell with a dimer comprising two single chain trimeric CD40L Fc fusion proteins of claim 13, wherein said single chain trimeric CD40L Fc fusion protein dimer activates the target cell upon binding;
optionally wherein the target cell is an antigen presenting cell;
optionally wherein the target cell is a B cell, dendritic cell, macrophage, monocyte, granulocyte, or eosinophil, or a combination thereof;
optionally wherein the target cell is a B cell;
optionally wherein the target cell is a dendritic cell;
optionally wherein the target cell is a macrophage;
optionally wherein upon activation, the target cell activates a second cell;
optionally wherein the second cell is a T cell, neutrophil, or a combination thereof; optionally wherein the second cell is CD4+ T cell, CD8+ T cell, mucosal associated invariant T (MAIT) cell, natural killer cell, neutrophil, or a combination thereof.

30. The method of claim 29, wherein the method is a method of activating a target cell, wherein the activation of the target cell is measured as:
(a) increased proliferation or maturation of the target cell;
optionally wherein proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%; or
(b) prolonged survival time of the target cell;
optionally wherein survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

31. The method of claim 29, wherein the contacting further comprises administering a pharmaceutical composition comprising a pharmaceutically acceptable carrier and the single chain trimeric CD40L Fc fusion proteins;
optionally wherein the contacting enhances an innate anti-neoplastic immune response.

32. A method of treating cancer in a subject comprising:
administering to the subject:
(a) a therapeutically effective amount of the polypeptide of claim 1,
(b) a therapeutically effective amount of the single chain trimeric CD40L Fc fusion protein of claim 13, or
(c) a therapeutically effective amount of a dimer comprising two single chain trimeric CD40L Fc fusion proteins of claim 13.

33. The method of claim 32, further comprising administering a pharmaceutical composition comprising a pharmaceutically acceptable carrier and the single chain trimeric CD40L Fc fusion proteins,
optionally wherein the treatment enhances an innate anti-neoplastic immune response.

34. The method of claim 32, further comprising co-administration of a second therapy.

35. The method of claim 32, wherein said cancer is selected from the group consisting of melanoma, mesothelioma, advanced solid tumor and lymphoma.

36. A method for promoting antibody production by a population of B cells, comprising:
(a) contacting the population of B cells with the polypeptide of claim 1, wherein said single chain trimeric CD40L fusion protein activates the B cells upon binding;
(b) contacting the population of B cells with the single chain trimeric CD40L Fc fusion protein of claim 13, wherein said single chain trimeric CD40L Fc fusion protein activates the B cells upon binding; or
(c) contacting the population of B cells with a dimer comprising two single chain trimeric CD40L Fc fusion proteins of claim 13, wherein said single chain trimeric CD40L Fc fusion protein dimer activates the B cells upon binding;
optionally wherein antibody production by the population of B cells is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

37. The method of claim 36, wherein the population of B cells is contacted in the presence of an antigen, and wherein the antibody produced by the B cells specifically binds to the antigen;
optionally wherein the method further promotes formation of memory B cells capable of producing the antibody in response to the antigen.

38. A method of increasing antigen presentation by a population of dendritic cells, comprising contacting the dendritic cells in the presence of the antigen with an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein a minimal percentage of dendritic cells presenting the antigen in the population of dendritic cells is increased by about 10%, about 20%, about 30%, about 40%, about 45%, about 50%, about 55%, about 60%, about 65%, about 70%, about 75%, about 80%, about 85%, about 90%, about 95%, or about 99%.

39. The method of claim 38, wherein the antigen presentation by the dendritic cells is measured by co-culturing dendritic cells labeled with a first fluorescent dye and the antigen labeled with a second fluorescent dye, wherein the first fluorescent dye and the second fluorescent dye are different;
optionally wherein the percentage of dendritic cells presenting the antigen is measured by determining the percentage of dendritic cells co-localizing with the antigen in the population of dendritic cells.

40. A method of increasing secretion of pro-inflammatory cytokines by a population of immune cells, comprising:
(a) contacting the population of immune cells with a population of antigen presenting cells in the presence of the polypeptide of claim 1, wherein said single chain trimeric CD40L fusion protein activates the antigen presenting cells upon binding;
(b) contacting the population of immune cells with a population of antigen presenting cells in the presence of the single chain trimeric CD40L Fc fusion protein of claim 13, wherein said single chain trimeric CD40L Fc fusion protein activates the antigen presenting cells upon binding; or
(c) contacting the population of immune cells with a population of antigen presenting cells in the presence of a dimer comprising two single chain trimeric CD40L Fc fusion proteins of claim 13, wherein said single chain trimeric CD40L Fc fusion protein dimer activates the antigen presenting cells upon binding;
wherein upon activation, the antigen presenting cells activate the population of immune cells;
optionally wherein the antigen presenting cells present an antigen to the population of immune cells;
optionally wherein the cytokine is IL-1, IL-2, IL-6, IL-12, IL-17, IL-22, IL-23, GM-CSF, TNF-α, IFN-γ, or any combination thereof;
optionally wherein the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%;
optionally wherein, upon activation of the antigen presenting cells, presentation of the antigen to the population of immune cells is increased;
optionally wherein a minimal percentage of antigen presenting cells presenting the antigen in the population of antigen presenting cells is increased by about 10%, about 20%, about 30%, about 40%, about 45%, about 50%, about 55%, about 60%, about 65%, about 70%, about 75%, about 80%, about 85%, about 90%, about 95%, or about 99%;
optionally wherein the antigen presenting cells comprise dendritic cells, macrophages, B cells, or a combination thereof; optionally wherein the population of immune cells comprise T cells, neutrophils, or a combination thereof.

41. The method of claim 36, wherein the polypeptide, the single chain trimeric CD40L Fc fusion protein, or the dimer is in a vaccine composition or adjuvant composition.

42. The method of claim 36, wherein the antigen is in a vaccine composition.

43. The method of claim 36, wherein the antigen is originated or derived from
(a) an infectious pathogen;
optionally wherein the infectious pathogen is a virus, a bacteria, a fungus, a parasite, or a combination thereof;
(b) a diseased cell;
(c) a cell infected by an infectious pathogen;
optionally wherein the infectious pathogen is a virus, a bacteria, a fungus, a parasite, or a combination thereof; or
(d) a cancer cell.

44. The method of claim 36, wherein the antigen is presented by an antigen presenting cell;
optionally wherein the antigen presenting cell is a dendritic cell;
optionally wherein the antigen is associated with an MHC class I or MHC class II complex.

45. A method of increasing phagocytosis of diseased cells by a population of macrophages, comprising:
contacting the diseased cells, the macrophages, or both the diseased cells and the macrophage with an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein a minimal percentage of phagocytotic macrophages in the population of macrophages is increased by about 10%, about 20%, about 30%, about 40%, about 45%, about 50%, about 55%, about 60%, about 65%, about 70%, about 75%, about 80%, about 85%, about 90%, about 95%, or about 99%;
optionally wherein the phagocytosis by macrophages is measured by co-culturing macrophages labeled with a first fluorescent dye and diseased cells labeled with a second fluorescent dye, wherein the first fluorescent dye and the second fluorescent dye are different.

46. The method of claim 45, wherein the percentage of phagocytotic macrophages is measured by determining the percentage of macrophages comprising the diseased cells;
optionally wherein the diseased cells are cancer cells or cell infected by an infectious pathogen;
optionally wherein the infectious pathogen is a virus, a bacteria, a fungus, a parasite, or a combination thereof.

47. A method of increasing expression of a CD40 polypeptide by a target cell, comprising:
contacting the target cell with an effective amount of (i) the polypeptide of claim 1, (ii) the single chain trimeric CD40L Fc fusion protein of claim 13, or (iii) a dimer comprising two single chain trimeric CD40L Fc fusion proteins of claim 13.

48. The method of claim 47, wherein the target cell is:
(a) a diseased cell,
(b) a cancer cell,
(c) a cell infected by an infectious pathogen;
optionally wherein the infectious pathogen is a virus, a bacteria, a fungus, a parasite, or a combination thereof; or
(d) a B cell, natural killer cell, dendritic cell, macrophage, monocyte, granulocyte, eosinophil, neutrophil, or a combination thereof;
optionally wherein the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99%.

49. A method of forming a pro-inflammatory milieu in a tissue surrounding a population of diseased cells, comprising contacting the tissue with an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein:
(a) infiltration of activated B cells, CD4+ T cells, CD8+ T cells, dendritic cells, macrophages, natural killer cells, monocytes, granulocytes, eosinophils and/or neutrophils in the tissue is increased;
(b) concentration of a pro-inflammatory cytokine is increased in the tissue;
optionally wherein the pro-inflammatory cytokine is IL-1, IL-2, IL-6, IL-12, IL-17, IL-22, IL-23, GM-CSF, TNF-α, IFN-γ, or any combination thereof;
(c) presentation of antigens originated or derived from the diseased cells by antigen presentation cells is increased in the tissue;
(d) phagocytosis of the diseased cells is increased in the tissue;
(e) apoptosis of the diseased cells induced by cell-mediated cytotoxicity is increased in the tissue;
(f) apoptosis of the diseased cells induced by antibody-dependent cellular cytotoxicity is increased in the tissue; and/or
(g) the population of the diseased cells is reduced in the tissue;
optionally wherein the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99% in the tissue.

50. A method of eliminating a diseased cell in a subject, comprising administering to the subject an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein:
(a) the diseased cell does not express a CD40 polypeptide;
(b) the diseased cell expresses a CD40 polypeptide;
(c) the diseased cell is a cancer cell; or
(d) the diseased cell is a cell infected by an infectious pathogen;
optionally wherein the infectious pathogen is a virus, a bacteria, a fungus, a parasite, or a combination thereof.

51. A method of treating cancer in a subject in need thereof, comprising administering to the subject an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein
(a) the treatment enhances an innate, humoral or cell-mediated anti-neoplastic immune response;
(b) the method further comprises co-administration of a second therapy; and/or
(c) the cancer is selected from the group consisting of melanoma, mesothelioma, advanced solid tumor and lymphoma.

52. A method of treating an infection in a subject in need thereof, comprising administering to the subject an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein:
(a) the treatment enhances an innate, humoral, or cell-mediated anti-infective immune response;
(b) the subject is co-administered with a vaccine composition for preventing the infection in the subject;
optionally wherein, the vaccine composition is co-administered simultaneously or sequentially.

53. A method of increasing the response to an antigen in a subject in need thereof, comprising administering to the subject an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein the antigen is an antigen of a cancer, tumor, pathogen, or allergen.

54. A method of increasing a response to a vaccine in a subject in need thereof, comprising administering to the subject the vaccine and an effective amount of (i) the polypeptide of claim 1, (ii) the single chain trimeric CD40L Fc fusion protein of claim 13, or (iii) a dimer comprising two single chain trimeric CD40L Fc fusion proteins of claim 13;
optionally wherein the vaccine is a vaccine against a tumor, cancer, pathogen or allergen.


SEQUENCE LISTING (40 sequences)

SEQ ID NO: 1 - protein, 672 aa, Artificial Sequence
  TPP000183311 - OVTSB10 - heavy_chain - hsctCD40LG2_EKWv2NPQ
NPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 687 aa, Artificial Sequence
  TPP000183310 - OVTSB9 - heavy_chain - hsctCD40LG2_EKWv2NPQ_G4S3
NPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGGSGGGGSVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 695 aa, Artificial Sequence
  TPP000182997 - C4LW30 - mature_protein - NPQ_G3S3_sthCD40L_G4S2_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 700 aa, Artificial Sequence
  TPP000182996 - C4LW29 - mature_protein - NPQ_G3S3_sthCD40L_G4S3_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 705 aa, Artificial Sequence
  TPP000182995 - C4LW28 - mature_protein - NPQ_G3S3_sthCD40L_G4S4_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 690 aa, Artificial Sequence
  TPP000182994 - C4LW27 - mature_protein - NPQ_G3S3_sthCD40L_G4S_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 685 aa, Artificial Sequence
  TPP000182993 - C4LW26 - mature_protein - NPQ_G3S3_sthCD40L_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 689 aa, Artificial Sequence
  TPP000182992 - C4LW25 - mature_protein - NPQ_sthCD40L_G4S2_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 694 aa, Artificial Sequence
  TPP000182991 - C4LW24 - mature_protein - NPQ_sthCD40L_G4S3_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 699 aa, Artificial Sequence
  TPP000182990 - C4LW23 - mature_protein - NPQ_sthCD40L_G4S4_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 684 aa, Artificial Sequence
  TPP000182989 - C4LW22 - mature_protein - NPQ_sthCD40L_G4S_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 679 aa, Artificial Sequence
  TPP000182988 - C4LW21 - mature_protein - NPQ_sthCD40L_IgG1_AAS
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 680 aa, Artificial Sequence
  TPP000182986 - C4LW19 - mature_protein - IgG1_AAS_G4S2_NPQ_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 14 - protein, 685 aa, Artificial Sequence
  TPP000182985 - C4LW18 - mature_protein - IgG1_AAS_G4S3_NPQ_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 15 - protein, 690 aa, Artificial Sequence
  TPP000182984 - C4LW17 - mature_protein - IgG1_AAS_G4S4_NPQ_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 16 - protein, 681 aa, Artificial Sequence
  TPP000182983 - C4LW16 - mature_protein - IgG1_AAS_G4S_NPQ_G3S3_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 17 - protein, 686 aa, Artificial Sequence
  TPP000182982 - C4LW15 - mature_protein - IgG1_AAS_G4S2_NPQ_G3S3_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 18 - protein, 691 aa, Artificial Sequence
  TPP000182981 - C4LW14 - mature_protein - IgG1_AAS_G4S3_NPQ_G3S3_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 19 - protein, 696 aa, Artificial Sequence
  TPP000182980 - C4LW13 - mature_protein - IgG1_AAS_G4S4_NPQ_G3S3_sthCD40L
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 143 aa, Artificial Sequence
  CD40L v.1
NPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKL

SEQ ID NO: 21 - protein, 141 aa, Artificial Sequence
  CD40L v.2
QIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKL

SEQ ID NO: 22 - protein, 143 aa, Artificial Sequence
  CD40L v.3
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 23 - protein, 9 aa, Artificial Sequence
  Linker
EGKSSGSGS

SEQ ID NO: 24 - protein, 15 aa, Artificial Sequence
  Linker
GGGGSGGGGSGGGGS

SEQ ID NO: 25 - protein, 12 aa, Artificial Sequence
  Linker
GGGSGGGSGGGS

SEQ ID NO: 26 - protein, 10 aa, Artificial Sequence
  Linker
GGGGSGGGGS

SEQ ID NO: 27 - protein, 20 aa, Artificial Sequence
  Linker
GGGGSGGGGSGGGGSGGGGS

SEQ ID NO: 28 - protein, 5 aa, Artificial Sequence
  Linker
GGGGS

SEQ ID NO: 29 - protein, 229 aa, Artificial Sequence
  Fc Monomer v.1
VERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 232 aa, Artificial Sequence
  Fc Monomer v.2
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 223 aa, Artificial Sequence
  Fc Monomer v.3
HTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 669 aa, Artificial Sequence
  TPP000161222 - C40W33 - mature_protein
IAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 140 aa, Artificial Sequence
  CD40L v.4
IAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 34 - protein, 141 aa, Artificial Sequence
  CD40L v.5
QIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 35 - protein, 443 aa, Artificial Sequence
  CD40L Trimer v.1
NPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKL

SEQ ID NO: 36 - protein, 453 aa, Artificial Sequence
  CD40L Trimer v.2
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 37 - protein, 447 aa, Artificial Sequence
  CD40L Trimer v.3
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 38 - protein, 440 aa, Artificial Sequence
  CD40L Trimer v.4
IAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEGKSSGSGSQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 39 - protein, 4 aa, Artificial Sequence
  Linker
  MISC_FEATURE (1)..(4): GGGS can be repeated n times, wherein n is an integer from 1 to 20
GGGS

SEQ ID NO: 40 - protein, 5 aa, Artificial Sequence
  Linker
  MISC_FEATURE (1)..(5): GGGGS can be repeated n times, wherein n is an integer from 1 to 20
GGGGS